(19) United States
(12) Patent Application Publication
(10) Pub. No.: US 2005/0239088 A1
(43) Pub. Date: Oct. 27, 2005

(54) Intron fusion proteins, and methods of identifying and using same

(76) Inventors: H. Michael Shepard, San Francisco, CA (US); Gail M. Clinton, Portland, OR (US); David B. Lackey, San Diego, CA (US); Pei Jin, Palo Alto, CA (US)

Correspondence Address: FISH & RICHARDSON, PC, 12390 EL CAMINO REAL, SAN DIEGO, CA, 92130-2081, US

(21) Appl. No.: 10/846,113

(22) Filed: May 14, 2004

Related U.S. Application Data
(60) Provisional application No. 60/471,141, filed on May 16, 2003.

(51) Int. Cl. C12Q 1/68; C07H 21/04; C12N 9/12; C12N 15/09

(52) U.S. Cl. 435/6; 435/69.1; 435/194; 435/320.1; 435/325; 536/23.2

(57) ABSTRACT

Isoforms of receptor tyrosine kinases, including intron fusion proteins and pharmaceutical compositions containing receptor tyrosine kinase isoforms, including intron fusion proteins, are provided herein. Methods of identifying and preparing isoforms of cell surface receptors including receptor tyrosine kinases are provided. Also provided are methods of treatment with cell surface receptor isoforms including intron fusion proteins of receptor tyrosine kinases.

### RELATED APPLICATIONS

This application claims the benefit of under 35 U.S.C. §119(e) priority to U.S. Provisional Application No. 60/471,141, to H. Michael Shepard, Gail M. Clinton and David B. Lackey, entitled “INTRON FUSION PROTEINS, AND METHODS OF IDENTIFYING AND USING SAME,” filed May 16, 2003. The subject matter of this application is incorporated in its entirety by reference thereto.

This application is related in subject matter to U.S. Provisional Application No. (attorney docket number 17118-P2817 (17118-008P01)), to Pei Jin, entitled “CELL SURFACE RECEPTOR ISOFORMS, AND METHODS OF IDENTIFYING AND USING SAME,” filed May 14, 2004. The subject matter of this application is incorporated in its entirety by reference thereto.

### FIELD OF THE INVENTION

Isoforms of receptor tyrosine kinases, including intron fusion proteins and pharmaceutical compositions containing receptor tyrosine kinase isoforms, including intron fusion proteins, are provided herein. Methods of identifying and preparing isoforms of cell surface receptors including receptor tyrosine kinases are provided. Also provided are methods of treatment with cell surface receptor isoforms including intron fusion proteins of receptor tyrosine kinases.

### BACKGROUND

Cell signaling pathways involve a network of molecules including polypeptides and small molecules that interact to relay extracellular, intercellular and intracellular signals. Such pathways can interact like a relay; handing off signals from one member of the pathway to the next. Modulation of one member of the pathway can be relayed through the signal transduction pathway, resulting in modulation of activities of other pathway members and modulating outcomes of such signal transduction such as affecting phenotypes and responses of a cell or organism to a signal. Diseases and disorders can involve misregulated or changes in modulation of signal transduction pathways. A goal of therapeutics is to target such misregulated pathways to restore more normal regulation in the signal transduction pathway.

Receptor tyrosine kinases (RTKs) are among the polypeptides involved in many signal transduction pathways. RTKs play a role in a variety of cellular processes, including cell division, proliferation, differentiation, migration and metabolism. RTKs can be activated by ligands. Such activation in turn activates events in a signal transduction pathway, such as by triggering autocrine or paracrine cellular signaling pathways, for example, activation of second messengers, which results in specific biological effects. Ligands for RTKs bind specifically to the cognate receptors.

RTKs have been implicated in a number of diseases including cancers such as breast and colorectal cancers, gastric carcinoma, gliomas and mesodermal-derived tumors. Misregulation of RTKs has been noted in several cancers. For example, breast cancer can be associated with upregulation of ErbB-2 (also reffered to as Her2) receptor. RTKs also have been associated with diseases of the eye, including diabetic retinopathies and macular degeneration. RTKs also are associated with regulating pathways involved in angiogenesis, including physiologic and tumor blood vessel formation. RTKs also are implicated in the regulation of cell proliferation, migration and survival.

Small molecules can be designed as therapeutics that target RTKs. There are a number of limitations with such strategies. Small molecules can be limited to interactions with one receptor and thus unable to address conditions where multiple family members can be misregulated. Small molecules also can be promiscuous and affect receptors other than the intended target. Additionally, some small molecules bind irreversibly to RTKs and the merits of such approaches have not been validated. Thus, there exists an unmet need for therapeutics for treatment of diseases, including cancers and other diseases involving undesirable cell proliferation and inflammatory reactions, involving RTK activity and/or the activity of other cell surface proteins. Accordingly, among the objects herein, it is an object to provide such therapeutics and methods for identifying or discovering candidate therapeutics.

### SUMMARY

Therapeutic molecules for treating diseases and disorders involving signal transduction pathways and other cell surface receptor interactions are provided. Also provided are compositions containing the molecules and methods for treating diseases and conditions with the compositions. Also provided are methods for identifying candidate therapeutics. In particular, cell surface receptor isoforms, families of CSR isoforms and methods of making CSR isoforms are provided herein. The cell surface isoforms and families of isoforms provided herein include isoforms of receptor tyrosine kinases. Also provided are pharmaceutical compositions containing CSR isoforms and methods of treatment for diseases and conditions by administering or expressing CSR isoforms. Methods of identifying and generating amino acids sequences of CSR isoforms and nucleotide sequences encoding CSR isoforms also are provided herein.

Provided herein are isolated polypeptides that are cell surface receptor isoforms. In one embodiment, an isolated polypeptide contain a sequence of amino acids that has at least 95% sequence identity with a sequence of amino acids set forth in any of SEQ ID NOs: 1, 3, 5-8, 12, 14-17, 19, and 22-25 and allelic variations thereof, where sequence identity is compared along the full length of each SEQ ID to the full length sequence of the isolated polypeptide. Each of SEQ ID NOs: 1, 3, 5-8, 12, 14-17, 19 and 22-25 is a receptor tyrosine kinase isoform. Such polypeptides include polypeptide contains the same number of amino acids as set forth in the SEQ ID to which it has identity. Such polypeptides also include polypeptides from a mammal, such as a rodent, a primate or a human.

Isolated polypeptides provided herein also include polypeptides with at least one domain of a receptor tyrosine kinase operatively linked to at least one amino acid encoded by an intron of a gene encoding the receptor tyrosine kinase. Exemplary receptor tyrosine kinases are DDR including DDR1, EPHA including EPHA1 and EPHA8, FGFR4, MET, PDGFRA, TEK, TIE. Isolated polypeptides provided also include polypeptides with at least one domain of a receptor tyrosine kinase operatively linked to at least one amino acid encoded by an intron of a gene encoding the receptor tyrosine kinase and that contain a sequence of amino acids of SEQ ID NOs: 1, 3, 4-8, 10, 12, 14-17, 19, 20, 21 or 22-25.

Also provided are isolated polypeptides that include a shortened receptor tyrosine kinase lacking at least all or part of a kinase domain and/or all or a part of a transmembrane domain, where the polypeptide has reduced kinase activity and/or is not membrane localized compared to the non-shortened receptor tyrosine kinase. Such polypeptides include polypeptides that modulates a biological activity of the receptor tyrosine kinase. Exemplary receptor tyrosine kinases include DDR, EPHA1, EPHA8, FGFR2, FGFR4, MET, PDGFRA, and TIE. Such isolated polypeptide include polypeptides with at least 95% sequence identity with a sequence of amino acids set forth in any of SEQ ID NOs: 1, 3, 4-8, 10, 11, 12, 14-17, 19, 20, 21 or 22-25; where sequence identity is compared along the full length of each SEQ ID to the sequence of the full length of the isolated polypeptide.

Also provided herein are isolated polypeptides that lack a receptor tyrosine kinase cytoplasmic domain. The isolated polypeptides contain an intron-encoded sequence of amino acids, where the intron is from a receptor tyrosine kinase gene or the intron is the intron-encoded sequence of any of SEQ ID NOs: 1-8 and 10-25. The receptor tyrosine kinase gene can be selected from DDR1, EGFR, ERBB3, FLT1, MET, PDGFRA, TEK and TIE. Such polypeptides also include polypeptides that further lack a transmembrane domain. Such polypeptides include polypeptides that modulate a biological activity of a receptor tyrosine kinase. The biological activity can be dimerization, homodimerization, heterodimerization, kinase activity, autophosphorylation of the receptor tyrosine kinase, transphosphorylation of the receptor tyrosine kinase, phosphorylation of a signal transduction molecule, ligand binding, competition with the receptor tyrosine kinase for ligand binding, signal transduction, interaction with a signal transduction molecule, membrane association and membrane localization.

Also provided herein are pharmaceutical compositions containing the isolated polypeptides provided and described herein. Pharmaceutical compositions provided herein include compositions containing a polypeptide where the polypeptide comprises a sequence of amino acids that has at least 95% sequence identity with a sequence of amino acids set forth in any of SEQ ID NOs: 1, 3, 4-8, 10, 12, 14-17, 19, 20, 21 and 22-25 and allelic variations thereof, where sequence identity is compared along the full length of each SEQ ID to the full length of the sequence of the isolated polypeptide and each of SEQ ID NOs: 1, 3, 4-8, 10, 11, 12, 14-17, 19, 20, 21 and 22-25 is a receptor tyrosine kinase isoform. Among the compositions provided herein are compositions containing an amount of the polypeptide effective for modulating a biological activity of a receptor tyrosine kinase including one or more of dimerization, homodimerization, heterodimerization, kinase activity, autophosphorylation of the receptor tyrosine kinase, transphosphorylation of the receptor tyrosine kinase, phosphorylation of a signal transduction molecule, ligand binding, competition with the receptor tyrosine kinase for ligand binding, signal transduction, interaction with a signal transduction molecule, membrane association and membrane localization. Such compositions include those that inhibit a biological activity of a receptor tyrosine kinase. The compositions also include those that contain a polypeptide that complexes with a receptor tyrosine kinase. Among the compositions provided herein, are compositions that modulate dimerization of a receptor tyrosine kinase, including compositions that modulate, for example, inhibit, homodimerization and/or heterodimerization of a receptor tyrosine kinase, compositions that inhibits or reduces phosphorylation of a receptor tyrosine kinase, including composition inhibits or reduces transphosphorylation or autophosphorylation of a receptor tyrosine kinase and/or phosphorylation of a signal transduction molecule, composition that compete with the receptor tyrosine kinase for ligand binding and compositions that reduce or inhibit receptor tyrosine kinase ligand binding.

Provided herein are nucleic acid molecules encoding the polypeptides provided and described herein. Among the nucleic acid molecules provided herein are those that contain an intron and an exon, where the nucleic acid molecule encodes an open reading frame that spans an exon intron junction the open reading frame terminates at a stop codon contained in the intron. Such nucleic acid molecules include those where the intron encodes one or more amino acids of the encoded polypeptide. Also included are nucleic acid molecules where the stop codon is the first codon in the intron. Such nucleic acid molecules can be operatively linked to a promoter. Also provided are vectors comprising the nucleic acid molecules and cell comprising the vectors and/or nucleic acid molecules.

Provided herein are methods of treating a disease or condition by administering a pharmaceutical composition, including any of the pharmaceutical compositions provided herein. Exemplary diseases or condition for treatment include cancers, inflammatory diseases, infectious diseases angiogenesis-related condition, cell proliferation-related conditions, immune disorders and neurodegenerative diseases. Additional diseases and conditions for treatment include rheumatoid arthritis, multiple sclerosis and posterior intraocular inflammation, uveitic disorders, ocular surface inflammatory disorders, neovascular disease, proliferative vitreoretinopathy, atherosclerosis, rheumatoid arthritis, hemangioma, diabetes mellitus, inflammatory bowel disease, Chrohn's disease, psoriasis, Alzheimer's disease, lupus, vascular stenosis, restenosis, inflammatoryjoint disease, atherosclerosis, urinary obstructive syndromes, and asthma. Cancers for treatment by the methods included carcinoma, lymphoma, blastoma, sarcoma, and leukemia, lymphoid malignancies, squamous cell cancer, small-cell lung cancer, non-small cell lung cancer, adenocarcinoma of the lung, squamous carcinoma of the lung, cancer of the peritoneum, hepatocellular cancer, gastric cancer, stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney/renal cancer, prostate cancer, vulval cancer, thyroid cancer, hepatic carcinoma, anal carcinoma, penile carcinoma, and head and neck cancer. Included in the methods provided herein are methods of treatment with a pharmaceutical composition inhibits angiogenesis, cell proliferation, cell migration, or tumor cell growth or tumor cell metastasis. Also provided are methods of treatment where the disease or condition is a viral or parasitic infection and include treatment of malaria. In particular, provided is a method for treatment of malaria where the pharmaceutical composition contains a polypeptide that has at least 95% sequence identity with a sequence of amino acids set forth in SEQ ID NO: 19.

Provided herein are methods of drug discovery for identifying candidate molecules that modulate the activity of a cell surface receptor. The methods include the steps of: a) selecting a set of expressed gene sequences encoding a cell surface receptor or a portion thereof; b) assembling the set of expressed gene sequences into an aligned set of sequences; c) selecting at least one member sequence of the aligned set that encodes a cell surface receptor isoform, wherein the isoform lacks at least one domain or a portion thereof sufficient to modulate a biological activity of the cell surface receptor compared to a wildtype or predominant form of the cell surface receptor; to identify a candidate molecule that modulates the cell surface receptor. The methods also include those that further include designating one or more introns and exons within the member sequences of the aligned set by comparing the aligned set with a reference gene sequence; and selecting at least one member sequence encoding an isoform, wherein the member sequence comprises at least one amino acid and/or a stop codon encoded within an intron, operatively linked to an exon. The methods include selecting member sequence(s) selected that contain a 5′ exon corresponding to a 5′ coding exon of the reference gene sequence, and/or that contain the addition of at least one amino acid or a stop codon operatively linked to an exon encoding a kinase domain and/or the addition of at least one amino acid or stop codon operatively linked to an exon encoding a transmembrane domain.

The methods include identifying candidate molecules that modulate the activity of a receptor tyrosine kinase. The methods also include identifying candidate molecules that are isoforms of a cell surface receptor. Such isoforms include C-terminal shoretedn form of the cell surface receptor, isoforms that lack a domain or portion thereof such as a kinase domain, a transmembrane domain or a combination thereof. The methods include identifying candidate molecules that dimerize with the cell surface receptor, candidate molecules that bind a ligand where the cell surface receptor binds the same ligand and candidate molecule that compete with the cell surface receptor for ligand binding. The methods also include identifying candidate molecules that inhibit phosphorylation of the cell surface receptor. The methods provided include identifying candidate molecules that are modified in a biological activity of a cell surface receptor, such as candidate molecules that reduced in the biological activity as compared to the wildtype or predominant form of the receptor. Exemplary biological activities include dimerization, kinase activity, signal transduction, ligand binding, membrane association and membrane localization. Also provided are polypeptides identified by any of the methods provided herein.

### BRIEF DESCRIPTION OF THE FIGURES

FIG. 1 depicts an alignment of the erbB2 genomic locus with expressed sequence tags (ESTs) and splice variants of erbB2.

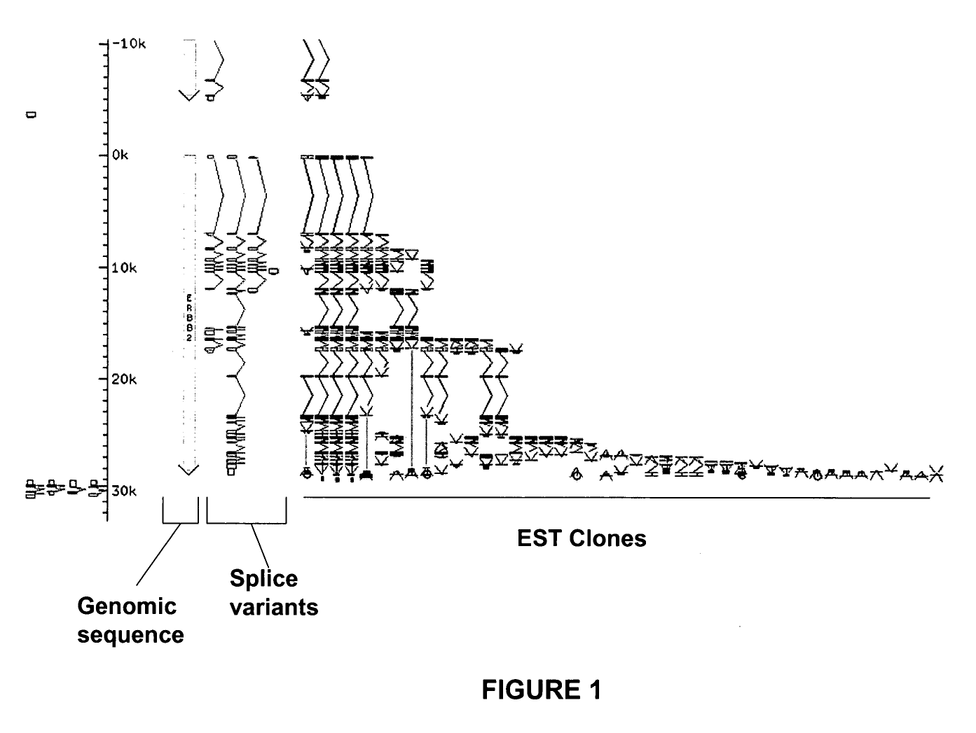

FIG. 2 depicts an alignment of the EphA8 genomic locus with expressed sequence tags (ESTs) and splice variants of EphA8.

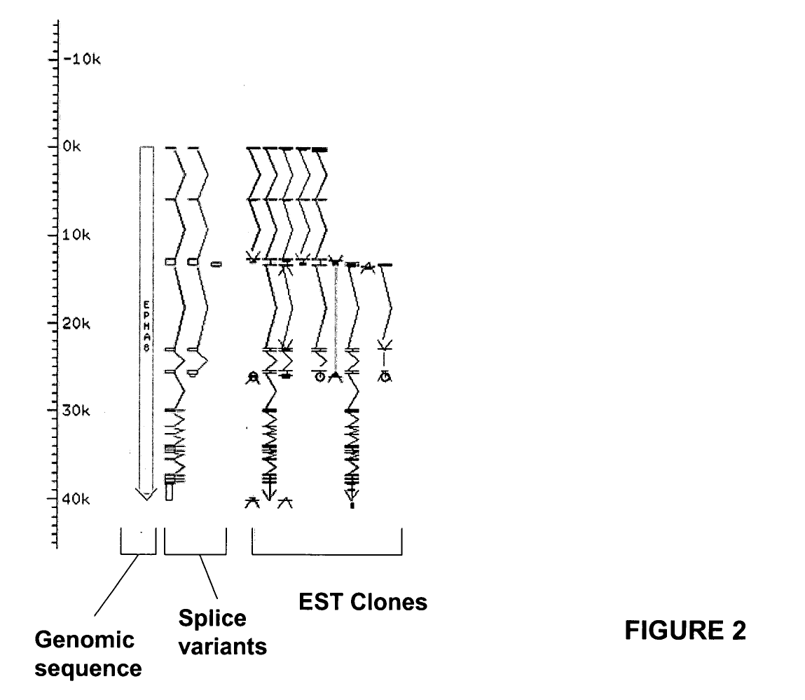

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a cell surface receptor is a protein that is expressed on the surface of a cell and typically includes at least one transmembrane domain or other moiety that anchors it to the surface of a cell. As a receptor, it can bind to ligands that mediate or participate in an activity of the cell surface receptor, such as signal transduction or ligand internalization. Cell surface receptors include, but are not limited to, receptor tyrosine kinases, such as growth factor receptors, and G-protein coupled receptors (GPCRs), such as ion channels.

As used herein, a receptor tyrosine kinase (RTK) refers to a protein, typically a glycoprotein, that is a member of the growth factor receptor family of proteins. Growth factor receptors are typically involved in cellular processes including cell growth, cell division, differentiation, metabolism and cell migration. RTKs also are known to be involved in cell proliferation, differentiation and determination of cell fate as well as tumor growth. RTKs have a conserved domain structure including an extracellular domain, a membrane-spanning (transmembrane) domain and an intracellular tyrosine kinase domain. Typically, the extracellular domain binds a polypeptide growth factor or a cell membrane-associated molecule. In some cases, an RTK does not bind a ligand, and/or is active independently from ligand binding; for example HER2 is active without ligand binding and a ligand binding HER2 has not been identified. Typically, the tyrosine kinase domain is involved in positive and negative regulation of the receptor. In some cases, for example ErbB3, kinase activity is not present in the receptor alone.

Receptor tyrosine kinases have been grouped into families based on, for example, structural arrangements of sequence motifs in their extracellular domains. For example, structural motifs such as, immunoglobulin, fibronectin, cadherin, epidermal growth factor and kringle repeats. Classification by structural motifs has identified greater than 16 families of RTKs, each with a conserved tyrosine kinase domain. Examples of RTKs include, but are not limited to, erythropoietin-producing hepatocellular (EPH) receptors, epidermal growth factor (EGF) receptors, fibroblast growth factor (FGF) receptors, platelet-derived growth factor (PDGF) receptors, vascular endothelial growth factor (VEGF) receptor, cell adhesion RTKs (CAKs), Tie/Tek receptors, insulin-like growth factor (IGF) receptors, and insulin receptor related (IRR) receptors. Exemplary genes encoding RTKs include, but are not limited to, ERBB2, ERBB3, DDR1, DDR2, TKT, EGFR, EPHA1, EPHA8, FGFR2, FGFR4, FLT1 (also known as VEGFR-1), FLK1 (also known as VEGFR-2) MET, PDGFRA, PDGFRB, and TEK (also known as TIE-2).

Dimerization of RTKs activates the catalytic tyrosine kinase domain of the receptor and tyrosine autophosphorylation. Autophosphorylation in the kinase domain maintains the tyrosine kinase domain in an activated state. Autophosphorylation in other regions of the protein influences interactions of the receptor with other cellular proteins. In some RTKs, ligand binding to the extracellular domain leads to dimerization of the receptor. In some RTKs, the receptor can dimerize in the absence of ligand. Dimerization also can be increased by receptor overexpression.

As used herein, an isoform of a cell surface receptor (also referred to herein as a CSR isoform), such as an isoform of a receptor tyrosine kinase, refers to a receptor which lacks a domain or portion thereof sufficient to alter a biological activity of the receptor or reduce a biological activity as compared to a wildtype and/or predominant form of the receptor. Generally, for purposes herein, a biological activity can be reduced in an isoform. Such reduction is at least 0.1, 0.5, 1, 2, 3, 4, 5, or 10-fold compared to a wildtype and/or predominant form of the receptor. Typically, a biological activity is altered 10, 20, 50, 100 or 1000-fold or more. In one embodiment, alteration of a biological activity is a reduction in the activity. With reference to an isoform, alteration of activity refers to difference in activity between the particular isoform, which is shortened, compared to the unshortened d form of the receptor. Alteration of biological activity includes an enhancement or a reduction of activity. In one embodiment, an alteration of a biological activity is a reduction in biological activity; the reduction can be at least 0.1 0.5 1, 2, 3, 4, 5, or 10 fold compared to a wildtype and/or predominant form of the receptor. Typically, a biological activity is reduced 5, 10, 20, 50, 100 or 1000 fold or more.

Reference herein to modulating the activity of a cell surface receptor means that a CSR isoform interacts in some manner with the receptor and activity, such as ligand binding or dimerization or other signal-transduction-related activity of the cell surface receptor is altered. Reference herein to a CSR isoform with altered activity refers to the alteration in an activity by virtue of the different structure or sequence of the CSR isoform compared to a cognate receptor.

A cell surface receptor isoform can be produced by any method known in the art including isolation of isoforms expressed in cells, tissues and organisms and by recombinant methods and by use of in silico and synthetic methods. Isoforms of cell surface receptors, including isoforms of receptor tyrosine kinases, can be encoded by alternatively spliced RNAs transcribed from a receptor tyrosine kinase gene. Such isoforms include exon deletion, exon retention, exon extension, exon truncation and intron retention alternatively spliced RNAs.

As used herein, exon deletion refers to an event of alternative RNA splicing that produces a nucleic acid molecule that lacks at least one exon as compared to an RNA encoding a wildtype or predominant form of a polypeptide.

As used herein, exon insertion refers to an event of alternative RNA splicing that produces a nucleic acid molecule that contains at least one exon not typically present in an RNA encoding a wildtype or predominant form of a polypeptide.

As used herein, exon extension refers to an event of alternative RNA splicing that produces a nucleic acid molecule that contains at least one exon that is greater in length (number of nucleotides contained in the exon) than the corresponding exon in an RNA encoding a wildtype or predominant form of a polypeptide. In some cases, as described further herein, an mRNA produced by exon extension encodes an intron fusion protein.

As used herein, exon truncation refers to an event of alternative RNA splicing that produces a nucleic acid molecule that contains a truncation of one or more exons such that the one or more exons are shorter in length (number of nucleotides) compared to a corresponding exon in an RNA encoding a wildtype or predominant form of a polypeptide.

As used herein, intron retention refers to an event of alternative RNA splicing that produces a nucleic acid molecule that contains an intron or a portion thereof operatively linked to one or more exons. In some cases, as described further herein, an mRNA produced by intron retention encodes an intron fusion protein.

As used herein, an Intron Fusion Protein (IFP) refers to an isoform that lacks one or more domain(s) or portion of one or more domain(s) resulting in an alteration of a biological activity of a receptor. In addition, an IFP contains one or more amino acids not encoded by an exon, operatively linked to exon-encoded amino acids and/or is shortened compared to a wildtype or predominant form encoded by a CSR gene. An IFP can be encoded by an alternatively spliced RNA and/or RNA molecules identified in silico by identifying potential splice sites and then producing such molecules by recombinant methods. Typically, an IFP is shortened by the presence of one or more stop codons in an IFP-encoding RNA that are not present in the corresponding sequence of an RNA encoding a wildtype or predominant form of a CSR polypeptide. Addition of amino acids and/or a stop codon can result in an IFP that differs in size and sequence from a wildtype or predominant form of a polypeptide.

IFPs for purposes herein include natural and combinatorial intron fusion proteins. A natural IFP refers to a polypeptide that is encoded by an alternatively spliced RNA that contains one or more amino acids encoded by an intron operatively linked to one or more portions of the polypeptide encoded by one or more exons of a gene. Alternatively spliced mRNA is one is isolated or is one that can be prepared synthetically by joining splice donor and acceptor sites in a gene. A natural IFP contains one or more amino acids and/or one or more stop codons encoded by an intron sequence. A combinatorial IFP refers to a polypeptide that is shortened compared to a wildtype or predominant form of a polypeptide. Typically, shortening removes one or more domains or a portion thereof from a polypeptide such that a biological activity is altered. Combinatorial IFPs often mimic a natural IFP in that one or more domains or a portion thereof that is/are deleted in a natural IFP derived from the same gene sequence or derived from a gene sequence in a related gene family.

As used herein, natural with reference to IFP, refers to any protein, polypeptide or peptide or fragment thereof (by virtue of the presence of the appropriate splice acceptor/donor sites) that is encoded within the genome of an animal and/or is produced or generated in an animal or that could be produced from a gene. Natural IFPs include allelic variant. IFPs can be modified post-translationally.

As used herein, an exon refers to a sequence of nucleotides that is transcribed into RNA and is represented in a mature form of RNA, such as mRNA (messenger RNA), after splicing and other RNA processing. An mRNA contains one or more exons operatively linked. Exons can encode polypeptides or a portion of a polypeptide. Exons also can contain non-translated sequences, for example, translational regulatory sequences. Exon sequences are often conserved and exhibit homology among gene family members.

As used herein, an intron refers to a sequence of nucleotides that is transcribed into RNA and is then typically removed from the RNA by splicing to create a mature form of an RNA, for example, an mRNA. Typically, nucleotide sequences of introns are not incorporated into mature RNAs, nor are intron sequences or a portion thereof typically translated and incorporated into a polypeptide. Splice signal sequences such as splice donors and acceptors are used by the splicing machinery of a cell to remove introns from RNA. It is noteworthy that an intron in one splice variant can be an exon (i.e., present in the spliced transcript) in another variant. Hence, spliced mRNA encoding an IFP can include an exon(s) and introns.

As used herein, splicing refers to a process of RNA maturation where introns in the mRNA are removed and exons are operatively linked to create a mature RNA. Alternative splicing refers to the process of producing multiple RNAs from a gene. Alternate splicing can include operatively linking less than all the exons of a gene, and/or operatively linking one or more alternate exons that are not present in all transcripts derived from a gene. Alternative RNA splicing can be regulated by developmental stage of an organism, cell or tissue type. In addition other factors, such as hormones and cytokines can modulate transcription and the resulting splicing patterns. These factors can produce different splicing patterns for an RNA within a cell or tissue type or stage, thus giving rise to different populations of RNAs, including mRNAs, tRNAs and rRNAs. Alternative splicing can give rise to RNAs and encoded molecules

As used herein, a gene, also referred to as a gene sequence, refers a sequence of nucleotides transcribed into RNA (introns and exons), including nucleotide sequence that encodes at least one polypeptide. A gene includes sequences of nucleotides that regulate transcription and processing of RNA. A gene also includes regulatory sequences of nucleotides such as promoters and enhancers, and translation regulation sequences.

As used herein, a splice site refers to one or more nucleotides within the gene that participate in the removal of an intron and/or the joining of an exon. Splice sites include splice acceptor sites and splice donor sites.

As used herein, a wildtype form, for example, a wildtype form of a polypeptide, refers to a polypeptide that is encoded by a gene. Typically a wildtype form refers to a gene (or RNA or protein derived therefrom) without mutations or other modifications that alter function or structure; wildtype forms include allelic variation among and between species.

As used herein, a predominant form, for example, a predominant form of a polypeptide, refers to a polypeptide that is the major polypeptide produced from a gene. A “predominant form” varies from source to source. For example, different cells or tissue types can produce different forms of polypeptides, for example, by alternative splicing and/or by alternative protein processing. In each cell or tissue type, a different polypeptide sequence can be a “predominant form.”

As used herein, a domain refers to a portion (a sequence of three or more, generally 5 or 7 or more amino acids) of a polypeptide that is a structurally and/or functionally distinguishable or definable. For example, a domain can be identified, defined or distinguished by homology of the sequence therein to related family members, such as homology and motifs that define an extracellular domain. In another example, a domain can be distinguished by its function, such as by enzymatic activity, e.g. kinase activity, or an ability to interact with a biomolecule, such as DNA binding, ligand binding, and dimerization. A domain independently can exhibit a biological function or activity such that the domain independently or fused to another molecule can perform a biological activity, such as, for example, proteolytic activity or ligand binding. A domain can be a linear sequence of amino acids or a non-linear sequence of amino acids from the polypeptide. Many polypeptides contain a plurality of domains. For example, receptor tyrosine kinases typically include, an extracellular domain, a membrane-spanning (transmembrane) domain and an intracellular tyrosine kinase domain.

As used herein, an allelic variant or allelic variation references to a polypeptide encoded by a gene that differs from a reference form of a gene (i.e. is encoded by an allele). Typically the reference form of the gene encodes a wildtype form and/or predominant form of a polypeptide from a population or single reference member of a species. Typically, allelic variants, which include variants between and among species typically, have at least 80%, 90% or greater amino acid identity with a wildtype and/or predominant form from the same species; the degree of identity depends upon the gene and whether comparison is interspecies or intraspecies. Generally, intraspecies alleleic variants have at least about 95% identity or greater with a wildtype and/or predominant form, including 96%, 97%, 98%, 99% or greater identity with a wildtype and/or predominant form of a polypeptide.

As used herein, modification in reference to modification of a sequence of amino acids of a polypeptide or a sequence of nucleotides in a nucleic acid molecule and includes deletions, insertions, and replacements of amino acids and nucleotides, respectively.

As used herein, an open reading frame refers to a sequence of nucleotides that encodes a polypeptide or a portion thereof. An open reading frame can encode a full-length polypeptide or a portion thereof. An open reading frame can be generated by operatively linking one or more exons or an exon and intron, when the stop codon is in the intron and all or a portion of the intron is in a transcribed mRNA.

As used herein, a polypeptide refers to two or more amino acids covalently joined. The terms “polypeptide” and “protein” are used interchangeably herein.

As used herein, shortened in reference to a shortened nucleic acid molecule or protein, refers to a sequence of nucleotides or amino acids that is less than full-length compared to a wildtype or predominant form of the protein or nucleic acid molecule.

As used herein, cognate receptor with reference to the isoforms provided herein refers to the receptor that is encoded by the same gene as the particular isoform. Generally, the cognate receptor also is a predominant form. For example, herstatin is encoded by a splice variant of the Her-2 receptor (erbb2 receptor). Thus, Her-2 is the cognate receptor for herstatin.

As used herein, a reference gene refers to a gene that can be used to map introns and exons within a gene. A reference gene can be genomic DNA or portion thereof that can be compared with, for example, an expressed gene sequence, to map introns and exons in the gene. A reference gene also can be a gene encoding a wildtype or predominant form of a polypeptide.

As used herein, a family or related family of proteins or genes refers to a group of proteins or genes, respectively that have homology and/or structural similarity and/or functional similarity with each other.

As used herein, a premature stop codon is a stop codon occurring in the open reading frame of a sequence before the stop codon used to produce or create a full-length form of a protein, such as a wildtype or predominant form of a polypeptide. The occurrence of a premature stop codon can be the result of, for example, alternative splicing and mutation.

As used herein, an expressed gene sequence refers to any sequence of nucleotides transcribed or predicted to be transcribed from a gene. Expressed gene sequences include, but are not limited to, cDNAs, ESTs, and in silico predictions of expressed sequences, for example, based on splice site predictions and in silico generation of spliced sequences.

As used herein, an expressed sequence tag (EST) is a sequence of nucleotides generated from an expressed gene sequence. ESTs are generated by using a population of mRNA to produce cDNA. The cDNAs can be produced for example, by priming from the polyA tail present on mRNAs. cDNAs also can be produced by random priming using one or more oligonucleotides which prime cDNA synthesis internally in mRNAs. The generated cDNAs are sequenced and the sequences are typically stored in a database. An example of an EST database in dbEST found online at ncbi.nlm.nih.gov/dbEST. Each EST sequence is typically assigned a unique identifier and information such as the nucleotide sequence, length, tissue type where expressed, and other associated data is associated with the identifier.

As used herein, a kinase is a protein that is able to phosphorylate a molecule, typically a biolmolecule, including macromolecules and small molecules. For example, the molecule can be a small molecule, a protein. Phosphorylation includes auto-phosphorylation. Some kinases have constitutive kinase activity. Other kinases require activation. For example, many kinases that participate in signal transduction are phosphorylated. Phosphorylation activates their kinase activity on another biomolecule in a pathway. Some kinases are modulated by a change in protein structure and/or interaction with another molecule. For example, complexation of a protein or binding of a molecule to a kinase can activate or inhibit kinase activity.

As used herein, designated refers to the selection of a molecule or portion thereof as a point of reference or comparison. For example, a domain can be selected as a designated domain for the purpose of constructing polypeptides which are modified within the selected domain. In another example, an intron can be selected as a designated intron for the purpose of identifying RNA transcripts that include or exclude the selected intron.

As used herein, modulate and modulation refer to a change of an activity of a molecule, such as a protein. Activities include, but are not limited to biological activities, such as signal transduction. Modulation can include an increase in the activity (i.e., up-regulation agonist activity), a decrease in activity (i.e., down-regulation or inhibitition) or any other alteration in an activity (such as periodicity, frequency, duration, kinetics. Modulation can be context-dependent and typically modulation is compared to a designated state, for example, the wildtype protein, the protein in a constitutive state, or the protein as expressed in a designated cell type or condition.

As used herein, inhibit and inhibition refer to a reduction in a biological activity.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof.

As used herein, a pharmaceutical effect refers to an effect observed upon administration of an agent intended for treatment of a disease or disorder or for amelioration of the symptoms thereof.

As used herein, treatment means any manner in which the symptoms of a condition, disorder or disease or other indication, are ameliorated or otherwise beneficially altered.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to animals, including mammals, such as human beings. As used herein, a patient refers to a human subject.

As used herein, a biological activity refers to a function of a polypeptide including but not limited to complexation, dimerization, multimerization, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and oncogenesis. A biological activity can be assessed by assays described herein and by standard assays known in the art, including but not limited to, in vitro assays, cell-based assays, in vivo assays, animal models and other known biological models.

As used herein, complexation refers to the interaction of two or more molecules such as two molecules of a protein to form a complex. The interaction can be by noncovalent and/or covalent bonds and includes, but is not limited to, hydrophobic and electrostatic interactions, Van der Waals forces and hydrogen bonds. Generally, protein-protein interactions involve hydrophobic interactions and hydrogen bonds. Complexation can be influenced by environmental conditions such as temperature, pH, ionic strength and pressure, as well as protein concentrations.

As used herein, dimerization refers to the interaction of two molecules of the same type, such as two molecules of a receptor. Dimerization includes homodimerization where two identical molecules interact. Dimerization also includes heterodimerization of two different molecules, such as two subunits of a receptor and dimerization of two different receptor molecules. Typically, dimerization involves two molecules that interact with each other through interaction of a dimerization domain contained in each molecule. As used herein, in silico refers to research and experiments performed using a computer. In silico methods include, but are not limited to, molecular modeling studies, biomolecular docking experiments, virtual representations of molecular structures and/or processes, such as molecular interactions, sequence alignments and comparisons such as by using BLAST, ACEMBLY, AND SIM4.

As used herein, biological sample refers to any sample obtained from a living or viral source and includes any cell type or tissue of a subject from which nucleic acid or protein or other macromolecule can be obtained. The biological sample can be a sample obtained directly from a biological source or processed For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants. Also included are soil and water samples and other environmental samples, viruses, bacteria, fungi, algae, protozoa and components thereof.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically or using recombinant molecular biology methods.

As used herein, a biomolecule is any compound found in nature, or derivatives thereof. Biomolecules include, but are not limited to: oligonucleotides, oligonucleosides, proteins, peptides, amino acids, peptide nucleic acids (PNAs), oligosaccharides and monosaccharides.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single- (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

As used herein, synthetic, in the context of a synthetic sequence and synthetic gene refers to a nucleic acid molecule that is produced by recombinant methods and/or by chemical synthesis methods.

Nucleotide analogs contained in a polynucleotide can be, for example, mass modified nucleotides, which allows for mass differentiation of polynucleotides; nucleotides containing a detectable label such as a fluorescent, radioactive, luminescent or chemiluminescent label, which allows for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. Nucleic acids Res. 25: 2792-2799 (1997)).

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, primer refers to an oligonucleotide containing two or more deoxyribonucleotides or ribonucleotides, generally more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

As used herein, production by recombinant means by using recombinant DNA methods means the use of the well-known methods of molecular biology for expressing proteins encoded by cloned DNA.

As used herein, “isolated,” with reference to molecule, such as a nucleic acid molecule, oligonucleotide, polypeptide or antibody, indicates that the molecule has been altered by the hand of man from how it is found in its natural environment. For example, a molecule produced by and/or contained within a recombinant host cell is considered “isolated.” Likewise, a molecule that has been purified, partially or substantially, from a native source or recombinant host cell, or produced by synthetic methods, is considered “isolated.” Depending on the intended application, an isolated molecule can be present in any form, such as in an animal, cell or extract thereof; dehydrated, in vapor, solution or suspension; or immobilized on a solid support.

As used herein, the term “vector” refers to a nucleic acid molecule capable of transporting another nucleic acid to which it has been linked. One type of vector is an episome, i.e., a nucleic acid capable of extra chromosomal replication. Vectors include those capable of autonomous replication and/or expression of nucleic acids to which they are linked. Vectors capable of directing the expression of genes to which they are operatively linked are referred to herein as “expression vectors.” In general, expression vectors are often in the form of “plasmids,” which are generally circular double-stranded DNA loops that, in their vector form are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, “transgenic animal” refers to any animal, generally a non-human animal, e.g., a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. This molecule can be stably integrated within a chromosome, i.e., replicate as part of the chromosome, or it can be extrachromosomally replicating DNA. In the typical transgenic animals, the transgene causes cells to express a recombinant form of a protein.

As used herein, a reporter gene construct is a nucleic acid molecule that includes a nucleic acid encoding a reporter operatively linked to a transcriptional control sequence. Transcription of the reporter gene is controlled by these sequences. The activity of at least one or more of these control sequences is directly or indirectly regulated by another molecule such as a cell surface protein, a protein or small molecule involved in signal transduction within the cell. The transcriptional control sequences include the promoter and other regulatory regions, such as enhancer sequences, that modulate the activity of the promoter, or control sequences that modulate the activity or efficiency of the RNA polymerase. Such sequences are herein collectively referred to as transcriptional control elements or sequences. In addition, the construct can include sequences of nucleotides that alter translation of the resulting mRNA, thereby altering the amount of reporter gene product.

As used herein, “reporter” or “reporter moiety” refers to any moiety that allows for the detection of a molecule of interest, such as a protein expressed by a cell, or a biological particle. Typical reporter moieties include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety, referred to herein as a “reporter gene,” can be expressed as a fusion protein with a protein of interest or under to the control of a promoter of interest.

As used herein, the phrase “operatively linked” generally means the sequences or segments have been covalently joined into one piece of nucleic acid such as DNA or RNA, whether in single- or double-stranded form. The segments are not necessarily contiguous, rather two or more components are juxtaposed so that the components are in a relationship permitting them to function in their intended manner. For example, segments of RNA (exons) can be operatively linked such as by splicing, to form a single RNA molecule. In another example, DNA segments can be operatively linked, whereby control or regulatory sequences on one segment control permit expression or replication or other such control of other segments. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression, a sequence of nucleotides and a regulatory sequence(s) are connected in such a way to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences, refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, the phrase “generated from a nucleic acid” in reference to the generating of a polypeptide, such as an isoform and IFP, includes the literal generation of a polypeptide molecule and the generation of an amino acid sequence of a polypeptide from translation of the nucleic acid sequence into a sequence of amino acids.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, regulatory region means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or as part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding sites (IRES) elements for the creation of multigene, or polycistronic, messages, polyadenylation signals to provide proper polyadenylation of the transcript of a gene of interest and stop codons and can be optionally included in an expression vector.

As used herein, the “amino acids,” which occur in the various amino acid sequences appearing herein, are identified according to their well-known, three-letter or one-letter abbreviations (see TABLE 1). The nucleotides, which occur in the various DNA fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§ 1.821-1.822, abbreviations for amino acid residues are shown in TABLE 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is defined to include the amino acids listed in the Table of Correspondence and modified and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions can be made in accordance with those set forth in TABLE 2 as follows:

TABLE 2

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (D) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative (or non-conservative) substitutions.

As used herein, “similarity” between two proteins or nucleic acids refers to the relatedness between the amino acid sequences of the proteins or the nucleotide sequences of the nucleic acids. Similarity can be based on the degree of identity and/or homology of sequences and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM JApplied Math 48:1073 (1988)).

As used herein, sequence identity compared along the full length of a polypeptide compared to another polypeptide refers to assessing the identity of amino acid sequence in a polypeptide along its full-length. For example, if a polypeptide A has 100 amino acids and polypeptide B has 95 amino acids, identical to amino acids 1-95 of polypeptide A, then polypeptide B has 95% identity when sequence identity is compared along the full length of a polypeptide A compared to full length of polypeptide B.

As used herein, homologous (with respect to nucleic acid and/or amino acid sequences) means about greater than or equal to 25% sequence homology, typically greater than or equal to 25%, 40%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence homology, the number of conserved amino acids is determined by standard alignment algorithm programs, and is used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two nucleic acid molecules have nucleotide sequences that are at least 60%, 70%, 80%, 85%, 90%, 95%, 96%, 97%, 98% or 99% “identical” or “homologous” can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequences. Identity at a level of 90% or more is indicative of the fact that, assuming for exemplification purposes a test and reference polypeptide length of 100 amino acids are compared. No more than 10% (i.e., 10 out of 100) amino acids in the test polypeptide differs from that of the reference polypeptide. Similar comparisons can be made between test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that a given nucleic acid molecule can serve as a “probe” and as a “primer.” A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application.

As used herein, an effective amount is the quantity of a therapeutic agent necessary for previsou, curing, ameliorating, arresting or partially arresting a symptom of a disease or disorder.

B. Cell Surface Receptor (CSR) Isoforms

Provided herein are cell surface receptor (CSR) isoforms, families of CSR isoforms and methods of preparing CSR isoforms. The CSR isoforms differ from the cognate receptors in that there are insertions and/or deletions and the resulting CSR isoforms exhibit a difference in one or more activities or functions compared to the cognate receptor. Such changes include a change in a biological activity, such as elimination of kinase activity, and/or elimination of all or part of a transmembrane domain. The CSR isoforms provided herein can be used for modulating the activity of a cell surface receptor. They also can be used as targeting agents for delivery of molecules, such as drugs or toxins or nucleic acids, to targeted cells or tissues.

A CSR isoform refers to a receptor that lacks a domain or portion of a domain sufficient to alter a biological activity of the receptor. Thus, an isoform differs from a wildtype and/or predominant form of the receptor, in that it lacks one or more biological activities of the receptor. Additionally, CSR isoforms can contain a new domain and/or biological function as compared to a wildtype and/or predominant form of the receptor. For example, intron-encoded amino acids can introduce a new domain or portion thereof into an isoform. Biological activities that can be altered include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway. Generally, a biological activity is altered in an isoform at least 0.1, 0.5, 1, 2, 3, 4, 5, or 10 fold as compared to a wildtype and/or predominant form of the receptor. Typically, a biological activity is altered 10, 20, 50, 100 or 1000 fold or more. For example, an isoform can be reduced in a biological activity.

CSR isoforms can also modulate an activity of a wildtype and/or predominant form of the receptor. For example, a CSR isoform can interact directly or indirectly with a CSR isoform and modulate a biological activity of the receptor. Biological activities that can be altered include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway.

A CSR isoform can interact directly or indirectly with a cell surface receptor to cause or participate in a biological effect, such as by modulating a biological activity of the cell surface receptor. A CSR isoform also can interact independently of a cell surface receptor to cause a biological effect, such as by initiating or inhibiting a signal transduction pathway. For example, a CSR isoform can initiate a signal transduction pathway and enhance or promote cell growth. In another example, a CSR isoform can interact with the cell surface receptor as a ligand causing a biological effect for example by inhibiting a signal transduction pathway that can impede or inhibit cell growth. Hence, the isoforms provided herein can function as cell surface receptor ligands in that they interact with the targeted receptor in the same manner that a cognate ligand interacts with and alters receptor activity. The isoforms can bind as a ligand but not necessarily to the ligand binding site and serve to block receptor dimerization. They act as ligands in the sense that they interact with the receptor. The CSR isoforms also can act by binding to ligands for the receptor and/or by preventing receptor activities, such as dimeriztion.

For example, a CSR isoform can compete with a CSR for ligand binding. A CSR isoform can act as a dominant negative inhibitor, for example, when complexed with a CSR. A CSR isoform can act as a dominant negative inhibitor or as a competitive inhibitor of a CSR, for example, by complexing with a CSR isoform and altering the ability of the CSR to multimerize (e.g, dimerize or trimerize) with other CSRs. A CSR isoform can compete with a CSR for interactions with other polypeptides and cofactors in a signal transduction pathway.

Pharmaceutical compositions containing one or more different CSR isoforms are provided. Also provided are methods of treatment of diseases and conditions by administering the pharmaceutical compositions or delivering a CSR isoform, such by administering a vector that encodes the isoform. Administration can be effected in vivo or ex vivo.

Methods of identifying and producing CSR isoforms and nucleic acid molecules encoding CSR isoforms are provided herein. Also provided are methods for expressing, isolating and formulating CSR isoforms.

Classes of CSR Isoforms

CSR isoforms are polypeptides that lack a domain or portion of a domain sufficient to remove or reduce a biological activity of the receptor. CSR isoforms can be generated by alternate splicing or by recombinant methods. CSR isoforms can be encoded by alternatively spliced RNAs. CSR isoforms also can be generated by recombinant methods and by use of in silico and synthetic methods.

Typically, a CSR isoform produced from an alternatively spliced RNA is not a predominant form of a polypeptide produced by a gene. In some instances, a CSR isoform can be a tissue-specific or developmental stage-specific polypeptide. Alternatively spliced RNAs that can encode CSR isoforms include, but are not limited to, exon deletion, exon retention, exon extension, exon truncation, and intron retention RNAs.

(a) Alternative Splicing and Generation of CSR Isoforms

Genes in eukaryotes include intron and exon portions that are transcribed by RNA polymerase into RNA products generally referred to as pre-mRNA. Pre-mRNAs are typically intermediate products that are further processed through RNA splicing and processing to generate a final messenger RNA (mRNA). Typically, a final mRNA, contains sequences of ribonucleotides obtained by splicing out introns. Boundaries of introns and exons are marked by splice junctions; sequences of nucleotides that are used by the splicing machinery of the cell as signals and substrates for removing introns and joining together exon sequences. Exons are operatively linked together to form a mature RNA molecule. Typically, one or more exons in an mRNA contain an open reading frame encoding a polypeptide. In many cases, an open reading frame can be generated by operatively linking two or more exons; for example, a coding sequence can span exon junctions and an open reading frame is maintained across the junctions.

RNAs, during processing and maturation also can undergo alternative splicing to produce a variety of mRNAs from a single gene. Alternatively spliced mRNAs can contain different numbers of and/or arrangements of exons. For example, a gene that has 10 exons can generate a variety of alternatively spliced mRNAs. Some mRNAs can contain all 10 exons, some with only 9, 8, 7, 6, 5 etc. In addition, products for example, with 9 of the 10 exons, can be among a variety of mRNAs, each with a different exon missing. Alternatively spliced mRNAs can contain additional exons, not typically present in an RNA encoding a predominant or wildtype form. Addition and deletion of exons includes addition and deletion, respectively of a 5′ exon, 3′exon and an exon internal in an RNA. Alternatively spliced RNAs also include addition of an intron or a portion of an intron operatively linked to or within an RNA. For example, an intron normally removed by splicing in an RNA encoding a wildtype or predominant form can be present in an alternatively spliced RNA. An intron or intron portion can be operatively linked within an RNA, such as between two exons. An intron or intron portion can be operatively linked at one end of an RNA, such as at the 3′ end of a transcript. In some examples, the presence of intron sequence within an RNA terminates transcription based on poly-adenylation sequences within an intron.

Alternative RNA splicing patterns can vary dependent upon the cell and tissue type. Alternative RNA splicing also can be regulated by developmental stage of an organism, cell or tissue type. In addition other factors, such as hormones and cytokines can modulate transcription and the resulting splicing patterns. For example, RNA splicing enzymes and polypeptides that regulate RNA splicing can be present at different concentrations in particular cell and tissue types and at particular stages of development. In some cases, a particular enzyme or regulatory polypeptide can be absent from a particular cell or tissue type or at particular stages of development and/or by virtue of environment, such as hormone and cytokine expression. These differences can produce different splicing patterns for an RNA within a cell or tissue type or stage, thus giving rise to different populations of RNAs, including mRNAs, tRNAs and rRNAs. Such complexity permits, for example, a number of protein products appropriate for particular cell types or developmental stages to be produced from a single gene.

Alternatively spliced mRNAs can generate a variety of different polypeptides, also referred to herein as isoforms. Such isoforms include polypeptides with deletions, additions and shortened forms compared to the wildtype or predominant form. For example, a portion of an open reading frame normally encoded by an exon can be removed in an alternatively spliced mRNA, thus resulting in a shorter polypeptide. An isoform can have amino acids removed at the N- or C-terminus or the deletion can be internal. An isoform can be missing a domain or a portion of a domain as a result of a deleted exon. Alternatively spliced mRNAs also can generate polypeptides with additional sequences. For example, a stop codon can be contained in an exon; when this exon is not included in an mRNA, the stop codon is not present and the open reading frame continues into the sequences contained in downstream exons. In such examples, additional open reading frame sequences add additional amino acid sequences to a polypeptide and can include addition of a new domain or a portion thereof.

(b) Intron Fusion Proteins

One class of isoforms is Intron Fusion Proteins (IFPs). An IFP is an isoform that lacks a domain or portion of a domain sufficient to remove or reduce a biological activity of a receptor. In addition, an IFP can contain one or more amino acids not encoded by an exon, operatively linked to exon-encoded amino acids and/or is shortened as compared to a wildtype or predominant form encoded by a CSR gene. Typically, an IFP is shortened by the presence of one or more stop codons in an IFP-encoding RNA that are not present in the corresponding sequence of an RNA encoding a wildtype or predominant form of a CSR polypeptide. Addition of amino acids and/or a stop codon can result in an IFP that differs in size and sequence from a wildtype or predominant form of a polypeptide.

An IFP is modified in one or more biological activities. For example, addition of amino acids in an IFP can add, extend or modify a biological activity as compared to a wildtype or predominant form of a polypeptide. For example, fusion of an intron encoded amino acid sequence to a protein can result in the addition of a domain with new functionality. Fusion of an intron encoded amino acid sequence to a protein also can modulate an existing biological activity of a protein, such as by inhibiting a biological activity, for example, inhibition of dimerization or inhibition of kinase activity.

IFPs include natural and combinatorial intron fusion proteins. A natural IFP is encoded by an alternatively spliced RNA that contains one or more introns or a portion thereof operatively linked to one or more exons of a gene. A natural IFP contains one or more amino acids encoded by an intron sequence and/or an IFP can be shortened as a result of one or more stop codons encoded by an intron sequence operatively linked to one or more exons. A combinatorial IFP is a polypeptide that is shortened as compared to a wildtype or predominant form of a polypeptide. Typically, shortening removes one or more domains or a portion thereof from a polypeptide. Combinatorial IFPs often mimic a natural IFP by deleting one or more domains or a portion thereof that are deleted in a natural IFP derived from the same gene sequence or derived from a gene sequence in a related gene family.

i. Natural IFPs

Natural IPs are generated from a class of alternatively spliced mRNAs that includes mRNAs that have incorporated intron sequence into mRNA as well as exon sequences, such as intron retention RNAs and some exon extension RNAs. The incorporated intron sequences can include one or more introns or a portion thereof. Such mRNAs can arise by a mechanism of intron retention. For example, a pre-mRNA is exported from the nucleus to the cytoplasm of the cell before the splicing machinery has removed one or more introns. In some cases, splice sites can be actively blocked, for example by cellular proteins, preventing splicing of one or more introns.

Retention of one or more introns or a portion thereof also can lead to the generation of isoforms referred to herein as natural IFPs. For example, an intron sequence can contain an open reading frame that is operatively linked to the exon sequences by RNA splicing. Intron-encoded sequences can add amino acids to a polypeptide, for example, at either the N- or C-terminus of a polypeptide, or internally within a polypeptide sequence. In some examples, an intron sequence also can contain one or more stop codons. An intron encoded stop codon that is operatively linked with an open reading frame in one or more exons can terminate a polypeptide sequence. Thus, an isoform can be produced that is shortened as a result of the stop codon. In some examples, an intron retained in an mRNA can result in the addition of one or more amino acids and a stop codon to an open reading frame, thereby producing an isoform that terminates with an intron encoded sequence.

Provided herein are natural IFPs that can be generated by intron retention including IFPs with addition of one or more domains or a portion of a domain encoded by an intron and IFPs with one or more domains or portion of a domain deleted. For example, an intron sequence can be operatively linked in place of an exon sequence that is typically within an mRNA for a gene. A domain or portion thereof encoded by the exon is thus deleted from and intron encoded amino acids are included in the encoded polypeptide.

In another example, an intron sequence is operatively linked in addition to the typically present exons in an mRNA. In one example, an operatively linked intron sequence can introduce a stop codon in-frame with exon sequences encoding a polypeptide. In another example, an operatively linked intron sequence can introduce one or more amino acids into a polypeptide. In some embodiments, a stop codon in-frame also is operatively linked with exon sequences encoding a polypeptide, thereby generating an mRNA encoding a polypeptide with intron-encoded amino acids at the C terminus.

In one example of a natural IFP, one or more amino acids encoded by an intron sequence are operatively linked at the C terminus of a polypeptide. For example, an IFP is generated from a nucleic acid sequence that contains one or more exon sequences at the 5′ end of an RNA followed by one or more intron sequences or a portion of an intron sequence retained at the 3′ end of an RNA. An IFP produced from such nucleic acid contains exon-encoded amino acids at the N-terminus and one or more amino acids encoded by an intron sequence at the C-terminus. In another example, an IFP is generated from a nucleic acid by operatively linking a stop codon encoded within an intron sequence to one or more exon sequences, thereby generating a nucleic acid sequence encoding shortened polypeptide.

ii. Combinatorial IFPs

IFPs also can be generated by recombinant methods and/or in silico and synthetic methods to produce polypeptides that are modified as compared to a wildtype or predominant form of a polypeptide. These IFPs also are known as combinatorial IFPs. Typically, combinatorial IFPs are shortened polypeptides as compared to a wildtype or predominant form. Shortening can remove one or more domains or a portion thereof.

Combinatorial IFPs often mimic a natural IFP by deleting one or more domains or a portion thereof that are deleted in a natural IFP derived from the same gene sequence or derived from a gene sequence in a related gene family. For example, as is described further herein, by aligning sequences of gene family members, intron and exon structures can be identified in the nucleic acid sequence as well as by identifying encoded protein domains. Recombinant nucleic acid molecules encoding polypeptides can be synthesized that contain one or more exons and an intron or portion thereof. Such recombinant molecules can contain one or more amino acids and/or a stop codon encoded by an intron, operatively linked to an exon, producing an IFP. Recombinant polypeptides also can be produced that contain a combinatorial IFP.

(c) Intron-Encoded Isoforms

Another CSR isoform is an intron-encoded isoform. An intron-encoded isoform contains an intron sequence or portions thereof from an isoform, such as a natural IFP. An intron-encoded isoform can interact with a wildtype form or predominant form of a polypeptide produced from the same gene as the intron-encoded isoform. An intron-encoded isoform can interact with a molecule in a signal transduction pathway that interact with a wildtype form or predominant form of a polypeptide produced from the same gene as the intron-encoded isoform. An intron-encoded isoform can be expressed or produced as a fusion with exon-encoded sequences. An intron-encoded isoform can be expressed or produced as a fusion with heterologous sequences such as by adding a starting methionine. Stop codons can be engineered in the encoding nucleic acid molecule to terminate an intron-encoded isoform within or at the end of the intron sequence.

(d) Isoforms Generated by Exon Modifications

CSR isoforms can be generated by modification of an exon relative to a corresponding exon of an RNA encoding a wildtype or predominant form of a CSR polypeptide. Exon modifications include alternatively spliced RNA forms such as exon truncations, exon extensions, exon deletions and exon insertions. These alternatively spliced RNAs can encode CSR isoforms which differ from a wildtype or predominant form of a CSR polypeptide by including additional amino acids and/or by lacking amino acid sequences present in a wildtype or predominant form of a CSR polypeptide.

Exon insertions are alternative spliced RNAs that contain at least one exon not typically present in an RNA encoding a wildtype or predominant form of a polypeptide. An inserted exon can operatively link additional amino acids encoded by the inserted exon to the other exons present in an RNA. An inserted exon also can contain one or more stop codons such that the RNA encoded polypeptide terminates as a result of such stop codons. If an exon containing such stop codons is inserted upstream of an exon that contains the stop codon used for polypeptide termination of a wildtype or predominant form of a polypeptide, a shortened polypeptide can be produced.

An inserted exon can maintain an open reading frame, such that when the exon is inserted, the RNA encodes an isoform containing an amino acid sequence of a wildtype or predominant form of a polypeptide with additional amino acids encoded by the inserted exon. An inserted exon can be inserted 5′, 3′ or internally in an RNA, such that additional amino acids encoded by the inserted exon are linked at the N terminus, C-terminus or internally, respectively in an isoform. An inserted exon also can change the reading frame of an RNA in which it is inserted, such that an isoform is produced that contains only a portion of the sequence of amino acids in a wildtype or predominant form of a polypeptide. Such isoforms can additionally contain amino acid sequence encoded by the inserted exon and also can terminate as a result of a stop codon contained in the inserted exon.

CSR isoforms also can be produced from exon deletion events. An exon deletion refers to an event of alternative RNA splicing that produces a nucleic acid molecule that lacks at least one exon as compared to an RNA encoding a wildtype or predominant form of a polypeptide. Deletion of an exon can produce a polypeptide of alternate size such as by removing sequences that encode amino acids as well as by changing the reading frame of an RNA encoding a polypeptide. An exon deletion can remove one or more amino acids from an encoded polypeptide; such amino acids can be N-terminal, C-terminal or internal to a polypeptide depending upon the location of the exon in an RNA sequence. Deletion of an exon in an RNA also can cause a shift in reading frame such that an isoform is produced containing one or more amino acids not present in a wildtype or predominant form of a polypeptide. A shift in reading frame also can result in a stop codon in the reading frame producing an isoform that terminates at a sequence different from that of a wildtype or predominant form of a polypeptide. In one example, a shift of reading frame produces an isoform that is shortened as compared to a wildtype or predominant form of a polypeptide. Such shortened isoforms also can contain sequences of amino acids not present in a wildtype or predominant form of a polypeptide.

CSR isoforms also can be produced by exon extension in an RNA. Exon extension is an event of alternative RNA splicing that produces a nucleic acid molecule that contains at least one exon that is greater in length (number of nucleotides contained in the exon) than the corresponding exon in an RNA encoding a wildtype or predominant form of a polypeptide. Additional sequence contained in an exon extension can encode additional amino acids and/or can contain a stop codon that terminates a polypeptide. An exon insertion containing an in-frame stop codon can produce a shortened isoform that terminates in the sequence of the exon extension. An exon insertion also can shift the reading frame of an RNA, resulting in an isoform containing one or more amino acids not present in a wildtype or predominant form of a polypeptide and/or an isoform that terminates at a sequence different from that of a wildtype or predominant form of a polypeptide. An exon extension can include sequences contained in an intron of an RNA encoding a wildtype or predominant form of a polypeptide and thereby produce an intron fusion protein.

CSR isoforms also can be produced by exon truncation. Exon truncations are RNAs containing a truncation of one or more exons such that the one or more exons are shorter in length (number of nucleotides) as compared to a corresponding exon in an RNA encoding a wildtype or predominant form of a polypeptide. An RNA with an exon truncation can produce a polypeptide that is shortened d as compared to a wildtype or predominant form of a polypeptide. An exon truncation also can result in a shift in reading frame such that an isoform is produced containing one or more amino acids not present in a wildtype or predominant form of a polypeptide. A shift in reading frame also can result in a stop codon in the reading frame producing an isoform that terminates at a sequence different from that of a wildtype or predominant form of a polypeptide.

Alternatively spliced RNAs including exon modifications can produce CSR isoforms that lack a domain or a portion thereof sufficient to reduce or remove a biological activity. For example, exon modified RNAs can encode shortened CSR polypeptides that lack a domain or portion thereof. Exon modified RNAs also can encode polypeptides where a domain is interrupted by inserted amino acids and/or by a shift in reading frame that interrupts a domain with one or more amino acids not present in a wildtype or predominant form of a polypeptide.

C. Receptor Tyrosine Kinase Isoforms

CSR isoforms provided herein include isoforms of receptor tyrosine kinases (RTKs), including receptor tyrosine kinase IFPs. The receptor tyrosine kinases (RTKs) are a large family of structurally related growth factor receptors. RTKs are involved in cellular processes including cell growth, differentiation, metabolism and cell migration. RTKs also are known to be involved in cell proliferation, differentiation and determination of cell fate. Members of the family include, but are not limited to, epidermal growth factor (EGF) receptors, platelet-derived growth factor (PDGF) receptors, fibroblast growth factor (FGF) receptors, insulin-like growth factor (IGF) receptors, nerve growth factor (NGF) receptors, vascular endothelial growth factor (VEGF) receptors, receptors to ephrin (termed Eph), hepatocyte growth factor (HGF) receptors (termed MET), TEK/Tie-2 (the receptor for angiopoietin-1), discoidin domain receptors (DDR) and others, such as Tyro3/Ax1.

Provided herein are RTK isoforms that are modified in one more domains of an RTK.such that they lack a domain of an RTK or a portion of a domain sufficient to remove or reduce a biological activity of an RTK. Also provided are RTK isoforms modified at one or more amino acids of an RTK sequence such as by deletion and/or addition of one more amino acids. Additional amino acids can add a new domain or a portion thereof. RTK isoforms can be modified in a biological activity including, but not limited to, dimerization, kinase activity, signal transduction, ligand binding, membrane association and membrane localization. RTK isoforms also can modulate a biological activity of an RTK.

1. RTK Domains and Biological Activities

RTKs have a conserved domain structure including an extracellular domain, a membrane-spanning (transmembrane) domain and an intracellular tyrosine kinase domain. The extracellular domain can bind a ligand, such as a polypeptide growth factor or a cell membrane-associated molecule. Some RTKs have been classified as orphan receptors, having no identified ligand. Some RTKs are classified as constitutive RTKs, active without ligand binding, for example ErbB2 (HER2) does not reqire a ligand for activity.

Typically, dimerization of RTKs activates the catalytic tyrosine kinase domain of the receptor and subsequent activities in signal transduction. RTKs can be homodimers or heterodimers. For example, PDGF is a heterodimer composed of a and subunits. VEGF receptors are homodimers. EGF receptors can be either heterodimers or homodimers. In another example, erbB3, in the presence of the ligand heregulin, heterodimerizes with other members of the ErbB family (EGFR family) such as ErbB2 and ErbB3. Many RTKs are capable of autophosphorylation when dimerized, such as by transphosphorylation between subunits. Autophosphorylation in the kinase domain maintains the tyrosine kinase domain in an activated state. Autophosphorylation in other regions of the protein can influences interaction of the receptor with other cellular proteins.

RTKs interact in signal transduction pathways. For example, RTKs, when activated can phosphorylate other signaling molecules. For example, EGFR interacts in signal transduction pathways involved in processes including proliferation, dedifferentiation, apoptosis, cell migration and angiogenesis. EGFR family members can recruit signaling molecules through protein:protein interactions; some interactions involve specific binding of signaling molecules to tyrosine phosphorylated sites on the receptor. For example, the Grb2/Sos complex can bind to phosphotyrosine sites on EGFR, in turn activating the Ras/Raf/MAPK signaling cascade, which influences cell proliferation, migration and differentiation. Other exemplary signally molecules include other RTKs, G-coupled receptors, integrins, phospholipase C, Ca 2+/calmodulin-dependent kinases, transcriptional activators, cytokines and other kinases.

2. Receptor Tyrosine Kinase Isoforms

RTK isoforms lack a domain or a portion of a domain of a receptor tyrosine kinase. Thus, an RTK isoforms differs from its cognate RTK in one or more biological activities. In addition, an RTK isoform can modulate a biological activity of an RTK, such as by interacting with an RTK directly or indirectly. Biological activities include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway.

RTK Isoform Structure and Activity

In one embodiment, an RTK isoform is modified in a kinase domain. For example, an RTK isoform contains a deletion of a kinase domain or a portion thereof. The deletion need not be a deletion of the entire domain, one or more amino acids can be deleted within the domain. The deletion can be at the N-terminus of the kinase domain, the C-terminus or internally within the domain. In another example, an RTK isoform contains addition of amino acids in a kinase domain. The addition of amino acids can be at the N-terminus of the domain, the C-terminus or anywhere internally within a kinase domain.

In one aspect of the embodiment, kinase activity of an RTK isoform is altered. For example, kinase activity of an RTK isoform is reduced or eliminated. In one example, substrate specificity of the kinase activity of an RTK isoform is altered. For example, an RTK isoform is capable of autophosphorylation but not phosphorylation of other polypeptides, such as polypeptides in a signal transduction pathway. In another example, an RTK isoform phosphorylates other polypeptides but is not capable of autophosphorylation. Kinase activity of an RTK isoform can be enhanced in activity. Kinase activity of an RTK isoform can be altered in regulation. For example, the kinase activity can be constitutively active or constitutively inactive, for example, unregulated by the addition of ligand, by receptor dimerization, by comlexation such as through protein:protein interactions, and/or by autophosphorylation.

In one embodiment, an RTK isoform is modified in a transmembrane domain. For example, an RTK isoform contains a deletion of a transmembrane domain or a portion thereof. The deletion can be at the N-terminus of a transmembrane domain, the C-terminus or internally within the domain. In another example, an RTK isoform contains addition of amino acids in a transmembrane domain. The addition of amino acids can be at the N-terminus of the domain, the C-terminus or anywhere internally within the transmembrane domain.

In one aspect of the embodiments, membrane association and/or localization of an RTK isoform is altered. For example, an RTK isoform can be a soluble protein (e.g. not membrane localized), where a wildtype or a predominant form of the RTK is membrane localized. For example, an RTK isoform can be secreted extracellularly or localized in the cytoplasm or internally within a cellular organelle. An RTK isoform can be altered in its membrane localization. For example, an RTK isoform can associate with internal membranes, such as membranes of cellular organelles, but not the cytoplasmic membrane. An RTK isoform can be reduced in its association with a membrane, such that the proportion of membrane associated protein is altered; for example, some of the protein is soluble and some is membrane associated. An RTK isoform also can be altered in the orientation with or within a membrane compared to the orientation of a wildtype or predominant form of an RTK. For example, more or less of the polypeptide can be embedded within the membrane. More or less of the polypeptide can be associated with either side of the cellular membrane. For example, orientation can be altered such that more of the RTK isoform is found in the cytoplasm or extracellularly compared to a wildtype or predominant form of an RTK.

In one embodiment, an RTK isoform is altered in its dimerization activity. For example, an RTK-isoform homodimerizes (i.e. an RTK isoform: RTK isoform complex) but does not heterodimerize or is reduced in heterodimerization with a wildtype or predominant form of an RTK derived from the same gene. In another example, an RTK-isoform does not homodimerize with itself, or is reduced in homodimerization activity but can heterodimerize with a wildtype or predominant form of an RTK from the same gene or a different gene. In another example, an RTK isoform is reduced in heterodimerization with RTKs from other genes but heterodimerizes with RTKs from the same gene.

In one embodiment, an RTK isoform is altered in its signal transduction activity. For example, an RTK isoform is altered in its association with other cellular proteins or cofactors in a signal transduction pathway. For example, an RTK isoform is altered in an interaction such as, but not limited to, an interaction with another RTK, a G-coupled receptor, an integrin, phospholipase C, a Ca²⁺/calmodulin-dependent kinase, a transcriptional activator or regulator, a cytokine and another kinase. In another example, an RTK isoform alters signal transduction of an RTK. For example, an RTKisoform interacts with an RTKand alters its activity in signal transduction, such as by inhbiting or by stimulating signal transduction by the RTK.

In one embodiment, an RTK isoform is altered in two or more biological activities. For example, an RTK isoform is altered in kinase activity and membrane association. In another example, an RTK isoform is altered in kinase activity and dimerization. In yet another example, an RTK isoform is altered in kinase activity, dimerization and membrane association. For example, an RTK isoform is modified in both a kinase domain and a transmembrane domain. In another example, insertion of addition of amino acids interrupts the kinase domain and transmembrane domains. In another embodiment, an RTK isoform is modified at a domain junction, or outside the linear sequence of amino acids for a domain and the modification alters a structure, such as the 3-dimensional structure of a domain such as a kinase domain, or a transmembrane domain.

Modulation of RTKs by RTK Isoforms

RTK isoforms can modulate or alter a biological activity of an RTK, such as by interacting directly or indirectly with an RTK. Biological activities include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway. In one embodiment, interaction of an RTK isoform with an RTK, inhibits an RTK biological activity. In another embodiment, interaction of an RTK isoform with an RTK,stimulates a biological activity of an RTK.

For example, an RTK isoform competes with an RTK for ligand binding. An RTK isoform can be employed as a “ligand sponge” to remove free ligand and thereby regulate or modulate the activity of an RTK. In another example, an RTK isoform acts as a dominant negative inhibitor when heterodimerized or complexed with an RTK, for example, by preventing trans-autophosphorylation. An RTK isoform that lack the protein kinase domain, or a portion thereof sufficient to alter kinase activity, can inhibit activation of an RTK in a trans dominant manner.

In one embodiment, an RTK isoform acts as a competitive inhibitor of RTK dimerization. For example, an RTK isoform interacts with an RTK and prevents that RTK from homodimerizing or from heterodimerizing. An isoform that inhibits receptor dimerization can modulate downstream signal transduction pathways, such as by complexing with the receptor and inhibiting receptor activation as down stream signaling. An RTK isoform also act as a competitive inhibitor of an RTK by competing directly with an RTK for interactions with other polypeptides and cofactors in a signal transduction pathway.

D. Methods for Identifying and Generating CSR Isoforms

CSR isoforms can be generated by analysis and identification of naturally occurring genes and expression products (RNAs) using the bioinformatics methods and algorithms disclosed herein, for example by identifying and generating natural IFPs. In addition, CSR isoforms, such as IFPs can be generated by producing combinations of naturally occurring amino acid sequences, using the methods provided herein, such as bioinformatics methods, for example by generating combinatorial IFPs. CSR isoforms also can be generated using cloning methods in combination with bioinformatics methods such as sequence alignments and domain mapping and selections.

1. Methods for Identifying and Generating IFP Sequences

The methods herein for identifying natural IFPs employ comparisons of expressed gene sequences with a sequence of a gene, such as a genomic DNA sequence. For example, one or more IFPs can be generated by identifying intron retention sequences from among a set of expressed gene sequences, where the intron retention sequences contain one or more intron sequences operatively linked to exon sequences. IFPs can be selected from the intron retention sequences by selecting those that encode a polypeptide with one or more amino acids or a stop codon operatively linked to exon-encoded sequences.

Intron retention sequences can be identified by any method known in the art for identifying or predicting intron and exon boundaries. For example, intron retention sequences can be identified by obtaining a set of expressed gene sequences and selecting a subset of expressed gene sequences corresponding to a gene sequence. The subset of sequences can be assembled into an aligned set of sequences based on identities of the expressed sequences as compared with each other. The subset also can be aligned with a gene sequence such as a genomic gene sequence. Comparison of the aligned set with a genomic DNA sequence of the gene can identify intron and exon boundaries of the aligned set. Alternatively, the aligned set of expressed sequences can be compared with a gene sequence such as a gene sequence encoding a full-length polypeptide, or a predicted gene sequence based on a major form of RNA or encoded protein. Intron and exon boundaries can be identified based on sequences which are present in one or more sequences of the aligned set and absent in the gene sequence. Sequences that retain one or more introns or a portion thereof, operatively linked to one or more exons are selected.

For example, in one embodiment of the method, alternative RNA splicing patterns for a particular gene can be determined by obtaining the sequence of all the expressed sequence tags (ESTs) for that gene, regardless of cell or tissue type, then assembling these sequence tags into a set of contigs by aligning identical sequences. Each alternatively spliced pre-mRNA can be represented by a unique sequence, for example, by mapping each of these sequences onto the DNA sequence of the gene using the BLAST algorithm (Basic Local Alignment Search Tool). In this way, the intron/exon boundaries of each alternatively spliced mRNA are identified in the ESTs and are precisely defined on the gene sequence.

Because ESTs have now been cloned and sequenced from an extremely large number and variety of cell and tissue types, these EST sequences contain an approximation to the complete RNA splicing pattern for any given gene for all cells and tissue types for which ESTs have been sequenced. Moreover, the number of EST sequences and the variety of cell and tissue types from which they are derived is expected to increase in the future, so that a representation of the complete set of alternatively spliced mRNA variants is approached. Thus, the methods herein can be used to derive IFPs from broad classes of proteins, and IFPs expressed in a wide variety of cell and tissue types.

In one embodiment, alternative RNA splicing patterns are obtained through access to the public domain AceView database program, available from NCBI (The National Center for Biotechnology Information, at hypertext transfer protocol (http), on the world wide web, at the URL “ncbi.nlm.nih.gov/IPB/Research/Acembly/index.html”). This program unambiguously maps ESTs and mRNAs as well as sequence assemblies. For example, this program has mapped 2,763,401 ESTs and 83,872 mRNAs from the public databases, as well as 18,000 NCBI RefSeq. Acembly (the AceView program that maps alternative splice forms) clusters these into 83,874 genes, with altogether 210,122 alternative transcript variants. 33,286 genes have at least one validated gt-ag or gc-ag spliced intron, and on average 4.6 alternatively spliced variants. A graphical representation of the alternatively spliced mRNAs from each gene is presented by the AceView program. In addition, the amino acid sequence from each mRNAs can be obtained from this program that predict the protein isoforms expressed or predicted to be expressed in nature for at least some cell or tissue type. Sequences are selected which contain one or more introns or a portion thereof operatively linked to one or more exons.

From intron retention sequences, IFPs are selected that encode a polypeptide with one or more amino acids or a stop codon derived from an intron or portion thereof, operatively linked to one or more exons. Polypeptide sequences can be generated from the nucleic acid sequences such as intron retention sequences by standard molecular biology and bioinformatics methods. Such methods identify open reading frames within nucleic acid sequences and generate amino acid sequence encoded by the nucleic acid. In some embodiments, IFPs contain deletion of one or more domains of a polypeptide and/or addition of a domain or portion thereof. Protein domains can be identified by any method known in the art. Many bioinformatics programs and methods exist for predicting domains or identifying protein domains, for example, based on amino acid sequence homology and/or structural predictions. IFPs can be selected with contain one or more domains or are deleted in one or more domains based on these domain predictions.

In one embodiment, the Protein Families Database (PFAM) is used to determine which part of each protein isoform primary amino acid sequence contains a protein domain or portion thereof. Pfam is a semi-automatic database of protein families and domains, and contains multiple protein alignments and profile-HMMs of these families. Pfam is a large collection of protein multiple sequence alignments and profile hidden Markov models that can be used to determine the domain composition of any sequence of amino acids. Pfam is available on the World Wide Web in the United Kingdom at the URL “sanger.cgb.ki.se/Pfam,” in Sweden at the URL “cgb.ki.se/Pfam/, in France (http) at the URL “pfamjouy.inra.fr,” and in the US (http) at the URL “pfam.wustl.edu.” Version 6.6 of Pfam contains 3071 families, which match 69% of proteins in SWISS-PROT 39 and TrEMBL 14 (Bateman, A. et al. (2002) Nucleic Acids Research 30(1): 276-28). Pfamidentifies the protein motifs present in each of the protein isoforms predicted by AceView.

IFPs can be identified and generated from any gene or class of genes provided that expressed gene sequence and a gene sequence for comparison (genomic gene sequence or other sequence as described herein) is available or can be generated. For example, IFPs can be identified and generated from cell surface receptors including, but not limited to, receptor tyrosine kinases, receptor serine/threonine kinases and cytokine receptors.

2. Identifying RTK-IFPs

An example of a class of cell surface receptor proteins useful for the identification of IFPs is the receptor tyrosine kinase (RTK) class of cell surface receptor. The RTK cell surface receptor genes are used here to demonstrate methods, such as bioinformatics methods, for identification of natural IFPs. Natural IFPs can be identified and generated for RTK cell surface receptor genes by identifying intron retention sequences from a set of expressed gene sequences, where the intron retention sequences contain one or more intron sequences operatively linked to exon sequences. RTK IFPs can be selected from the intron retention sequences by selecting those that encode a polypeptide with one or more amino acids or a stop codon operatively linked to exons encoding RTK gene sequences. In one embodiment, RTK IFPs are identified that contain a first coding exon or a portion of the first coding exon of an RTK gene or a predicted RTK gene. Such RTK IFPs contain an N-terminal sequence with a domain or portion of a domain identical to a full length or wildtype RTK. In another embodiment RTK IFPs are selected in which at least one designated domain or a portion thereof is deleted, where the designated domain is contained by a full-length or wildtype RTK. In one example, the designated domain is a kinase domain. In another embodiment, the designated domain is a transmembrane domain.

In one exemplary embodiment, disclosed herein, an RTK IFPs contains an extracellular domain, but lacks an intracellular protein kinase domain. In another embodiment, an RTK IFP contains an extracellular domain and a transmembrane domain but lacks an intracellular protein kinase domain. A transmembrane domain is apparently dispensable, at least in the case of herstatin, but can contribute substantially to the apparent binding affinity of IFPs for their corresponding native receptor protein. Isoforms lacking an intracellular protein kinase domain, located at the protein C-terminus of RTKs, and/or transmembrane domain, are readily identifiable by using any domain localization, structural identification or homology based tools known in the art, for example, by applying the Pfam program/database to the alternative protein isoforms sequences.

Herstatin

An example of an RTK-IFP is herstatin, an IFP produced from the HER-2 gene (see U.S. Pat. No. 6,414,130 and U.S. Published Application No. 20040022785). The HER-2 (erbB-2) gene encodes a receptor tyrosine kinase that has been implicated as an oncogene and its role in human carcinomas has been investigated. HER-2 has a major mRNA transcript 4.5 kB that encodes a polypeptide of about 185 kD (P185HER2). P185HER2 contains an extracellular domain, a transmembrane domain and an intracellular domain with tyrosine kinase activity.

Other polypeptide forms are produced from the HER-2 gene and include polypeptides generated by proteolytic processing and forms generated from alternatively spliced RNAs. Herstatin (U.S. Pat. No. 6,414,130) is an alternatively spliced variant of the human epidermal growth factor receptor 2 (ERBB2) that is found in fetal kidney and liver, and includes a 79 amino acid intron-encoded insert at the C terminus. Herstatin contains subdomains I and II of the human epidermal growth factor receptor extracellular domain and a novel C-terminal domain encoded by an intron. The resulting herstatin protein contains 419 amino acids (340 amino acids from subdomains I and II, plus 79 amino acids from intron 8). The herstatin protein lacks extracellular domain IV, as well as the transmembrane domain and kinase domain. Herstatin has been shown to inhibit tyrosine kinase receptors of the ErbB family.

In an exemplary embodiment of the methods, the ERBB2 gene was used to identify IFPs. ERBB2 can be used as a control experiment, since herstatin derives from this gene as an alternative RNA splice form, and the amino acid sequence of this protein isoform has been determined from the alternative mRNA sequence. Using the method for detecting natural IFPs, ESTs from erbB2 and a genomic sequence of erbB2 were aligned. Aligned sequences were selected which contained at least one intron or a portion thereof operatively linked to one or more exons. Aligned sequences were further chosen where the encoded polypeptide contained one or more amino acids and/or a stop codon encoded by the intron sequence. From these aligned sequences, and based on domain mapping of the erbB2 sequence (e.g. using Pfam for domain mapping), a subset of sequences were chosen that lacked at least a portion of the erbB2 tyrosine kinase domain. A selected sequence matched the predicted the 419 amino acid herstatin protein isoform (Doherty et al. (1999) Proc. Natl. Acad. Sci. USA 90:10869-10874).

3. Generating Combinatorial IFPs

Combinatorial IFPs can be generated by assembling intron-encoded sequences such that they are operatively linked with exon sequences. Combinatorial IFPs include IFP polypeptides that do not occur in nature but can be assembled using predictions of intron/exon boundaries and intron and exon sequences. Combinatorial IFPs also include IFPs assembled by combining protein domains from different genes and/or assembling protein domains in a different order than is found in naturally occurring forms. Combinatorial IFPs also include IFPs, modified by altering one or more amino acids in specific protein regions to modify a biological activity of an IFP. Such modifications include modifying natural and combinatorial IFPs.

Combinatorial IFPs can be created by methods herein including mimicking the effects of intron retention by generating polypeptide sequences which lack one or more domains or a portion thereof of a full-length or wildtype function. Combinatorial IFPs can generate polypeptide isoforms that are altered in a biological activity as compared to a full length or wildtype protein.

Combinatorial IFPs can be generated in receptor tyrosine kinases (RTKs) which lack one or more domains or a portion thereof. Combinatorial RTK IFPs include combinatorial IFPs containing an extracellular domain and transmembrane domain but lacking an intracellular tyrosine kinase domain. Combinatorial RTK IFPs also include combinatorial IFPs containing an extracellular domain but lacking an intracellular tyrosine kinase domain and transmembrane domain.

In an exemplary embodiment, combinatorial IFPs are generated for TIE-2 tyrosine receptor kinase. A combinatorial IFP can be created from this gene by identifying domains of the gene using any domain prediction tool, such as described herein. For example PFAM can be used to identify the protein kinase domain of the TIE-2 gene using the public domain Acembly program available from NCBI (National Center for Biotechnology Information. Protein kinase, extracellular and transmembrane domains are identified in TIE-2. A polypeptide is constructed that deleted the intracellular kinase domain or a portion thereof, such as by deleting residues 839-1107, or a portion thereof. For example, a TIE-2 combinatorial IFP is constructed containing only residues 1-838. This polypeptide contains all extracellular receptor domains necessary for binding ligand, as well as any transmembrane domains, but lacks the protein kinase domain. Further TIE-2 combinatorial IFPs can be constructed which contain deletions within the extracellular and transmembrane domains. For example, TIE IFP 632, TIE IFP 533, TIE IFP 428, TIE IFP 344, TIE IFP 255, TIE IFP 197. Each polypeptide contains N terminal amino acids 1-x as denoted in the name TE IFP X. Such combinatorial IFPs can be tested for an IFP biological activity, for example, by determining the efficiency of inhibition of TIE-2 phosphorylation.

4. Methods of Identifying and Isolating CSR Isoforms

Provided herein are methods for identifying and isolating CSR isoforms that utilize cloning of expressed gene sequences and alignment with a gene sequence such as a genomic DNA sequence. For example, one or more isoforms can be isolated by selecting a candidate gene, such as a receptor tyrosine kinase. Expressed sequences, such as cDNAs or regions of cDNAs, are isolated. Primers can be designed to amplify a cDNA or a region of a cDNA. In one example, primers are designed which overlap or flank the start codon of the open reading frame of a candidate gene and primers are designed which overlap or flank the stop codon of the open reading frame. Primers can be used in PCR such as reverse transcriptase PCR (RT-PCR) with mRNA to amplify nucleic acid molecules encoding open reading frames. Such nucleic acid molecules can be sequenced to identify those which encode an isoform. In one example, nucleic acid molecules with different sizes (e.g. molecular masses) from the predicted size (such as a size predicted for encoding a wildtype or predominant form) are chosen as candidate isoforms. Such nucleic acid molecules can then be analyzed as described below to further select isoform-encoding molecules.

Computational analysis is performed using the obtained nucleic acid sequences to further select candidate isoforms. For example, cDNA sequences are aligned with a genomic sequence of a selected candidate gene. Such alignments can be performed manually or by using bioinformatics programs such as SIM4, a computer program for analysis of splice variants. Sequences with canonical donor-acceptor splicing sites (e.g. GT-AG) are selected. Molecules can be chosen which represent alternatively spliced products such as exon deletion, exon retention, exon extension and intron retention can be selected.

Sequence analysis of isolated nucleic acid molecules also can be used to further select isoforms that retain or lack a domain and/or biological function as compared to a wildtype or predominant form. For example, isoforms encoded by isolated nucleic acid molecules can be analyzed using bioinformatics programs such as described herein to identify protein domains. Isoforms can then be selected which retain or lack a domain or a portion thereof.

In one embodiment of the method, isoforms are selected which lack a transmembrane domain or portion thereof sufficient to lack or significantly reduce membrane localization. For example, isoforms are selected that are shortened before a transmembrane domain or that are shortened within a transmembrane domain. Isoforms also can be selected that lack a transmembrane domain or portion thereof and have one or more amino acids operatively linked in place of the missing domain or portion of a domain. Such isoforms can be the result of alternative splicing events such as exon extension, intron retention, exon deletion and exon insertion. In some case, such alternatively spliced RNAs alter the reading frame of an RNA and/or operatively link sequences not found in an RNA encoding a wildtype or predominant form. Isoforms also can be selected that lack a kinase domain or portion thereof. Isoforms can be selected that lack a kinase domain or portion thereof and also lack a transmembrane domain or portion thereof. Isoforms selected by the method include IFPs and intron-encoded isoforms.

For example, nucleic acid molecules encoding candidate RTK isoforms can be further selected for isoforms that lack a kinase domain, a transmembrane domain, an extracellular domain or a portion thereof. Nucleic acid molecules can be selected which encode an RTK isoform and have a biological activity that differs from a wildtype or predominant form of an RTK. In one example, RTK isoforms are selected that lack a transmembrane domain such that the isoforms are not membrane localized and are secreted from a cell.

5. Allelic Variants of Isoforms

Allelic variants of CSR isoform sequences, including natural and combinatorial IFPs can be generated or identified in nucleic acids from different species, populations or individuals of the same species. Such variants typically differ in one or more amino acids from the wildtype or predominant form in a tissue or cell source but are encoded by the corresponding gene in the cell, tissue or organism. Consequently, corresponding isoforms (or shortened variants or IFPs) differ from the reference protein in the same positions. For example, isoforms can be derived from different alleles of a gene; each allele can have one or more amino acid differences. Such alleles can have conservative and/or non-conservative amino acid differences. Allelic variants also include isoforms produced or identified from different subjects, such as individual patients or model animals. Amino acid changes can result in modulation of an isoform biological activity. In some cases, an amino acid difference can be “silent,” having no detectable effect on a biological activity. Allelic variants of isoforms also can be generated by mutagenesis. Such mutagenesis can be random or directed. For example, allelic variant isoforms can be generated that alter amino acid sequences or a potential glycosylation site to effect a change in glycosylation of an isoform, including alternate glycosylation, increased or inhibition of glycosylation at a site in an isoform. Allelic variant isoforms are at least 90% identical in sequence to an isoform. Generally, an allelic variant isoform is at least 95%, 96%, 97%, 98%, 99% identical to a reference isoform, typically an allelic variant is 98%, 99%, 99.5% identical to an isoform.

E. Exemplary RTK Isoforms

The methods herein can be used to identify, discover or generate CSR isoforms, such as CSR IFPs from a variety of genes. One exemplary group of genes to which the methods can be applied is receptor tyrosine kinases. Receptor tyrosine kinases (RTKs) constitute a large collection of polypeptides and the encoding genes that are grouped into families based on, for example, structural arrangements of sequence motifs in the polypeptides. For example, structural motifs in the extracellular domains such as, immunoglobulin, fibronectin, cadherin, epidermal growth factor and kringle repeats are used to group RTKs. Such classification by structural motifs has identified greater then 16 families of RTKs, each with a conserved tyrosine kinase domain. Examples of RTKs include, but are not limited to, erythropoietin-producing hepatocellular (EPH) receptors, epidermal growth factor (EGF) receptors, fibroblast growth factor (FGF) receptors, platelet-derived growth factor (PDGF) receptors, vascular endothelial growth factor (VEGF) receptors, cell adhesion RTKs (CAKs), Tie/Tek receptors, hepatocyte growth factor (HGF) receptors (termed MET), TEK/Tie-2 (the receptor for angiopoietin-1), discoidin domain receptors (DDR), insulin-like growth factor (IGF) receptors, insulin receptor-related (IRR) receptors and others, such as Tyro3/Ax1. Exemplary genes encoding RTKs include, but are not limited to, ERBB2, ERBB3, DDR1, DDR2, TKT, EGFR, EPHA1, EPHA8, FGFR2, FGFR4, FLT1 (also known as VEGFR-1), MET, PDGFRA, PDGFRB, and TEK (also known as TIE-2) and genes encoding the RTKS noted above and not set forth.

RTKs participate in signal transduction pathways and regulate critical cellular processes including cell proliferation, dedifferentiation, apoptosis, cell migration and angiogenesis. RTK activation and thus subsequent activation of a signal transduction pathway is generally dependent on receptor activation, such as by activation of the receptor by ligand binding and autophosphorylation. RTKs can be subject to misregulation leading to misregulation of signal transduction. Alternatively, certain RTKs are expressed on cells and lead to or participate in alteration in cellular activities, such as oncogenic transformation. Such expression and/or misregulation is associated with a number of diseases and conditions, including but not limited to diseases involving abnormal cell proliferation, such as neoplastic diseases, restenosis, disease of the anterior eye, cardiovascular diseases, obesity and a variety of others.

RTK isoforms provided herein and generated by methods provided herein can be used to modulate a biological activity of an RTK, such as an RTK endogenous to a particular cell type or tissue. The ability to modulate a biological activity of an RTK allows re-regulation of an RTKs as well as directed regulation of cellular pathways in which RTKs participate. Modulating a biological activity of an RTK includes direct modulation, whereby an RTK isoform interacts with an RTK, such as by complexation with an RTK, modulation of homodimerization and/or heterodimerization of an RTK and/or modulation of trans-phosphorylation of an RTK, including inhibition of phosphorylation of an RTK. Modulation of an RTK also includes indirect modulation whereby an RTK isoform indirectly affects a biological activity of an RTK. Indirect modulation includes isoforms that act as a “ligand sponge,” competing for ligand binding with an RTK. Indirect modulation also includes interactions of an isoform with signaling molecules in a signaling pathway, thus modulating the activity such as by competition with interactions of such signaling molecules with an RTK. Exemplary RTK isoforms and uses of such RTK isoforms in targeting and regulating RTK activity are described below.

1. EGFR

EGFR is a 170 kDa protein that binds to EGF, a small, 53 amino acid protein-ligand that stimulates the proliferation of epidermal cells and a wide variety of other cell types. EGF receptors are widely expressed in epithelial, mesenchymal and neuronal tissues and play important roles in proliferation and differentiation. EGF receptors are encoded by a family of related genes known also as erbb genes (e.g. erbB2, erbB3, erbB4) and HER genes (e.g. Her-2). The EGF receptor family includes four members, EGF-receptor (HER-1; erbb-1), human epidermal growth factor receptor-2 (HER-2; erbB-2), HER-3 (erbB-3) and HER-4 (erbB-4). The ligand for EGFR/HER-1 is EGF, while the ligand for HER-2, HER-3 and HER-4 is neuregulin-1 (NRG-1). NRG-1 preferentially binds to either HER-3 or HER-4 after which the bound receptor subunit heterodimerizes with HER-2. HER-4 also is capable of homodimerization to form an active receptor.

Misregulation of the ErbB family has been implicated in a number of different types of cancer. For example, overexpression of EGFR is associated with a number of human tumors including, but not limited to, esophageal, stomach, bladder and colon cancers, gliomas and meningiomas, squamous carcinoma of the lungs, and ovarian, cervical and renal carcinomas. Using the methods provided herein, RTK isoforms and pharmaceutical compositions containing RTK isoforms can be generated for use as therapeutic agents which target and re-regulate misregulation of EGF receptors.

In an exemplary embodiment, RTK isoforms were identified and generated using the methods provided herein for RTK-IFPs using EGF receptor genes erbB2 and erbB3. Isoforms identified by the method include RTK-IFPs set forth in SEQ ID NOS: 5-10.

a. ErbB-2

ErbB-2 is a member of the EGF receptor family. A ligand that binds with high affinity has not been identified for ErbB2. Instead, ErbB-3 or ErbB-4 when bound by ligand (NRG-1) heterodimerize with ErbB-2 to form an active receptor dimer. In addition, ErbB2 exhibits constitutive activity (homodimerization and kinase activity) in the absence of ligand. In addition, overexpression of ErbB-2 is capable of cell transformation. ErbB-2 overexpression has been identified in a variety of cancers, including breast, ovarian, gastric and endometrial carcinomas. Thus, targeting ErbB-2 homodimers can regulate ErbB-2 homodimerization. For example, an erbB-2 RTK isoform can target and down-regulate ErbB-2 overexpression. Additionally, an erbB-2 RTK-isoform can target erbB-3 and/or erbB-4 through heterodimerization.

Exemplary erbB-2 isoforms include erbB-2 IFPs set forth in SEQ ID NOS: 5-9. ErbB-2 isoforms can be used to modulate RTKs such as in the treatment of cancers characterized by the overexpression of EGFR receptors such as those characterized by overexpression of erbB-2 and/or erbB-3. For example, erbB-2 isoforms can be used as a treatment for autoimmune diseases which involve EGFR family members in the maintenance of inflammation and hyperproliferation, including asthma. ErbB-2 isoforms also can be used to target RTKs in conditions including Menetrier's disease, Alzheimer's disease and as modulators, for example as an antagonist for bone resorption.

b. ErbB-3

ErbB-3 also is a member of the EGF receptor family involved in regulating development of neuronal survival and synaptogenesis, astrocytic differentiation and microglial activation. The ligand for ErbB-3 is NRG-1. Although NRG-1 can bind both ErbB-3 and ErbB-4, ErbB-3 binds NRG-1 with an affinity an order of magnitude lower than ErbB-4. ErbB-3 has lower tyrosine kinase activity as compared to other members of the EGFR family. It is capable of recruiting alternative signaling molecules, for example, phosphatidylinositol-3 kinase. ErbB-3 overexpression has been implicated in a number of human cancers such as breast, lung and bladder cancers and adenocarcinomas.

Exemplary erbB-3 isoforms include the erbB-3 IFP set forth in SEQ ID NO: 10. ErbB-3 isoforms can be used to target RTKs such as in the treatment of cancers characterized by the overexpression of EGFR receptors such as those characterized by overexpression of erbB-2 and/or erbB-3. ErbB-3 isoforms can target erbB-3 homodimers. ErbB-3 isoforms can target erbB-2 through heterodimerization of an erbB-3 isoform with erbB-2. ErbB-3 isoforms can be used for treatment of diseases and conditions in which EGFR receptors are involved. For example, erbB-3 isoforms can be used as a treatment for autoimmune diseases which involve EGFR family members in the maintenance of inflammation and hyperproliferation, including asthma. ErbB-3 isoforms also can be used to target RTKs in conditions including Menetrier's disease, Alzheimer's disease and as modulators, for example as an antagonist for bone resorption.

2. Discoidin Domain Receptors—DDR1

Discoidin domain receptors (e.g. DDR-1) are a novel family of RTKs that are thought to play a role in cell adhesion. DDRs possess a unique structural motif in their extracellular domains that is homologous to the Dictyostelium discoideum (slime mold) protein discoidin-1, a carbohydrate-binding protein involved in cell aggregation. The discoidin-like domain contains approximately 160 amino acids and although not found in other RTKs, it is found in other extracellular molecules that are known to interact with cellular membrane proteins (such as, e.g., coagulation factors V and VIII). Collagen (e.g. collagens type I to type VI) stimulates DDR-1 autophosphorylation.

DDR tyrosine kinases have been linked to human cancers. For example, DDR1 can bind collagen and mediate collagen-induced activation of matrix metalloproteinase-1. Matrix metalloproteinase-1 is involved in the degradation of extracellular matrix, which allows neoplastic cells to metastasize. Overexpression of DDR-1 has been linked to cancers such as breast, ovarian and esophageal cancers and a variety of central nervous system neoplasms, such as pediatric brain cancers. Activation of DDR1 also has been implicated in inflammatory responses.

An exemplary DDR isoform is the DDR1-IFP and is set forth in SEQ ID NO: 1. DDR-1 isoforms can be used to modulate DDR-1 RTK. For example, a DDR-1 isoform can be used to down regulate DDR-1 overexpression and or activation in diseases and conditions in which DDR-1 is involved.

3. Eph Receptors

Eph receptors are the largest known family of RTKs. The ligands for Eph receptors are ephrins (Eph receptor interacting protein). Both ligand and receptor are membrane-bound molecules and signaling can occur through either protein. Ephs are characterized by a cytoplasmic tyrosine kinase domain, a conserved cysteine-rich domain, two fibronectin type III domains and an immunoglobulin-like N-terminal domain. Ephrins can either be GPI-linked (type A) or transmembrane proteins (type B). The Eph family of RTKs are involved in a variety of cellular processes, including embryonic patterning, neuronal targeting, vascular development and angiogenesis. Particularly due to a role in angiogenesis, Eph receptors have been implicated in human cancers, such as breast cancer. Misregulation of EphA receptors also are involved in pathological conditions. For example, upregulation of the EphA receptor tyrosine kinase stimulates vascular endothelial cell growth factor (VEGF)-induced angiogenesis, common in certain eye diseases, rheumatoid arthritis and cancer. An EphA isoform, such as an isoform acting as an EphA receptor antagonist can be used to block or inhibit inappropriate angiogenesis.

a. EphA1

EphA1 is a type A Eph receptor. Type A Eph receptors bind to type A ephrins, which are linked to cell membranes via a GPI anchor. EphA1 is expressed widely in differentiated epithelial cells, including skin, adult thymus, kidney and adrenal cortex. Overexpression of EphA1 has been implicated in a variety of human cancers, including head and neck cancer. EphA1 isoforms can be used to target such diseases and other conditions in which Eph receptors have been implicated. An exemplary EphA1 isoform is the Eph A1 IFP set forth in SEQ ID NO: 3.

b. EphA8

EphA8 is a type A Eph receptor. Type A Eph receptors bind to type A ephrins, which are linked to cell membranes via a GPI anchor. EphA8 has been implicated in cell migration and cell adhesion as well as nervous system development, including axon guidance. EphA8 isoforms can be used to target such diseases and other conditions in which Eph receptors have been implicated. An exemplary Eph A8 isoform is the EphA8 IFP set forth in SEQ ID NO: 4.

4. Fibroblast Growth Factor Receptors

The fibroblast growth factor receptor family includes FGFR-1, FGFR-2, FGFR-3, FGFR-4 and FGFR-5. There are at least 23 known FGF proteins that are capable of binding to one or more FGF receptors. FGF receptors are structurally characterized by three N-terminal Ig-like domains (extracellular), a transmembrane domain and two kinase domains at the C-terminus (cytoplasmic). FGFs and their receptors are involved in stimulation of cellular proliferation, promoting angiogenesis and wound healing, and modulating cell motility and differentiation. FGFRs have been implicated in a variety of human cancers as well as diseases of the eye.

a. FGFR-2

FGFR-2 is a member of the fibroblast growth factor receptor family. Ligands to FGFR-2 include a number of FGF proteins, such as, but not limited to, FGF-1 (basic FGF), FGF-2 (acidic FGF), FGF-4 and FGF-7. FGF receptors are involved in cell-cell communication of tissue remodeling during development as well as cellular homeostasis in adult tissues. Overexpression of, or mutations in, FGFR-2 have been associated with hyperproliferative diseases, including a variety of human cancers, including breast, pancreatic, colorectal, bladder and cervical malignancies. SEQ ID NO: 11 sets forth an exemplary FGF-2 isoform. FGF-2 isoforms such as FGF-2 IFPs can be used to treat conditions in which FGF is upregulated, including cancers.

b. FGFR-4

FGFR-4 is a member of the FGF receptor tyrosine kinase family. FGFR4 regulation is modified in some cancer cells. For example, in some adenocarcinomas FGFR4 is down-regulated as compared with expression in normal fibroblast cells. Alternate forms of FGFR4, are expressed in some tumor cells. For example, ptd-FGFR-4 lacks a portion of the FGFR4 extracellular domain but contains the third Ig-like domain, a transmembrane domain and a kinase domain. This isoform is found in pituitary gland tumors and is tumorigenic. FGFR4 isoforms can be used to treat diseases and conditions in which FGFR4 is misregulated. For example, an FGFR4-isoform can be used to down regulate tumorigenic FGFR4 isoforms such as ptd-FGFR4. An exemplary isoform is the FGFR4—IFP is set forth in SEQ ID NO: 12.

5. Platelet-Derived Growth Factor Receptors

Platelet-derived growth factor receptors are homo or heterodimers comprised of two subunits, α and β. Receptor subunits are comprised of five Ig-like domains at the N-terminus, a transmembrane domain, and a split kinase domain at the C-terminus. Similar to its receptor, PDGF ligand is a homo- or heterodimer of A and/or B chains. The α-PDGF receptor can be activated by either PDGF-A or PDGF-B. A β-PDGF receptor only can be activated by the PDGF-B chain. Two additional members of the PDGF family also have been isolated, PDGF-C and PDGF-D.

PDGF receptors and ligands are involved in a variety of cellular processes, including clot formation, extracellular matrix synthesis, chemotaxis of immune cells apoptosis and embryonic development. Overexpression of PDGF receptors has been linked to a number of human carcinomas, including stomach, pancreas, lung and prostate. Activation of the platelet derived growth factor receptor (PDGFR) is associated with benign prostatic hypertrophy and prostate cancer as well as other cancer types. Activation of PDGF-R also is associated with smooth muscle proliferation in development of atherosclerosis. PDGFR also has been implicated in modulating proliferative vitreoretinopathy, a common medical problem caused by the proliferation of fibroblastic cells behind the retina, resulting in retinal detachment.

Exemplary PDGFR isoforms are the PDGFR-IFPs set forth in SEQ ID NOS: 20 and 21. PDGFR isoforms can be used to target diseases and conditions in which PDGFR is involved, including hyperproliferative diseases, such as proliferative vitreoretinopathy and smooth muscle hyperproliferative conditions including atherosclerosis.

6. MET (HGF)

MET is a RTK for hepatocyte growth factor (HGF), a multifunctional cytokine controlling cell growth, morphogenesis and motility. HGF, a paracrine factor produced primarily by mesenchymal cells, induces mitogenic and morphogenic changes, including rapid membrane ruffling, formation of microspikes, and increased cellular motility. Signaling through MET can increase tumorigenicity, induce cell motility and enhance invasiveness in vitro and metastasis in vivo. MET signaling also can increase the production of protease and urokinase, leading to extracellular matrix/basal membrane degradation, which are important for promoting tumor metastasis.

MET is a RTK that is highly expressed in hepatocytes. MET is comprised of two disulfide-linked subunits, a 50-kD a subunit and a 145-kD β subunit. In the fully processed MET protein, the α subunit is extracellular, and the β subunit has extracellular, transmembrane, and tyrosine kinase domains. The ligand for MET is hepatocyte growth factor (HGF). Signaling through FGF and MET stimulates mitogenic activity in hepatocytes and epithelial cells, including cell growth, motility and invasion. As with other RTKs, these properties link MET to oncogenic activities. In addition to a role in cancer, MET also has been shown to be a critical factor in the development of malaria infection. Activation of MET is required to make hepatocytes susceptible to infection by malaria, thus MET is a prime target for prevention of the disease.

SEQ ID NO: 19 sets forth an exemplary MET isoform, a MET-IFP. MET isoforms can be used in treating or preventing metastatic cancer, and in inhibiting angiogenesis, such as angiogenesis necessary for tumor growth. The therapeutic applications of MET isoforms include lung cancer, malignant peripheral nerve sheath tumors (MPNST), colon cancer, gastric cancer, and cutaneous malignant melanoma.

MET isoforms also can be used in combination with other anti-angiogenesis drugs to prevent tumor cell invasiveness. Anti-angiogenesis drugs produce a state of hypoxia in tumors which can promote tumor cell invasion by sensitizing cells to HGF stimulation. MET isoforms can target and modulate biological activity of MET, such as by inhibiting or down-regulating MET when anti-angiogenesis drugs are given, thus preventing or inhibiting tumor cell invasiveness as well as by penetration of the tumor by new endothelial cells

Therapeutic applications of MET isoforms also include prevention of malaria. Plasmodium, the causative agent of malaria, must first infect hepatocytes to initiate a mammalian infection. Sporozoites migrate through several hepatocytes, by breaching their plasma membranes, before infection is finally established in one of them. Wounding of hepatocytes by sporozoite migration induces the secretion of hepatocyte growth factor (HGF), which renders hepatocytes susceptible to infection. Infection depends on activation of the HGF receptor, MET, by secreted HGF. The malaria parasite exploits MET as a mediator of signals that make the host cell susceptible to infection. HGF/MET signaling induces rearrangements of the host-cell actin cytoskeleton that are required for the early development of the parasites within hepatocytes. MET-isoforms can be administered as a therapeutic to down-regulate MET, thus inhibiting or preventing induction of MET signaling by malaria parasite and therefore inhibiting or preventing malaria infection. MET also can be used in vaccination against malaria, by preventing infection by sporozoites in the immediate post-vaccination period

7. FLT1 (VEGF-1R)

The vascular endothelial growth factor (VEGF) is a family of closely related growth factors with a conserved pattern of eight cysteine residues and sharing common VEGF receptors. VEGF receptors include VEGFR-1 (Flt-1) and VEGFR-2 (Flk-1/KDR). Ligands for VEGF receptors include vascular endothelial growth factor-A (also known as vasculotropin (VAS) or vascular permeability factor (VPF)), VEGF-B, VEGF-C, VEGF-D and placental growth factor (PIGF). The VEGF proteins and receptors play an important role in many aspects of angiogenesis, including cell migration, proliferation and tube formation, thus linking these proteins to the pathogenesis of many types of cancer. Flt-1 and Flk are two genes encoding VEGFR family members.

Flt-1 (fins-like tyrosine kinase-1) is a member of the VEGF receptor family of tyrosine kinases. Ligands for Flt-1 include VEGF-A and P1GF (placental growth factor). Since Flt-1 and its ligands are important for angiogenesis, misregulation of these proteins have significant impacts on a variety of diseases stemming from abnormal angiogenesis, such as proliferation or metastasis of solid tumors, rheumatoid arthritis, diabetic retinopathy, retinopathy and psoriasis. Flt-1 also has been implicated in Kawasaki disease, a systemic vasculitis with microvascular hyperpermeability.

Exemplary RTK-isoforms for targeting VEGFR-related diseases and conditions include VEGFR-IFPs set forth in SEQ ID NOS: 13-18. Such isoforms can be used in the treatment of acute inflammatory disease, such as Kawasaki disease, rheumatoid arthritis, diabetic retinopathy, retinopathy and psoriasis, as well as re-regulation of abnormal angiogenesis. Additionally VEGFR-isoforms can be used for treatment of cancers including breast carcinoma.

8. TEK (TIE-2)

Tie-1 and Tie-2/TEK are endothelial RTKs with immunoglobulin and epidermal growth factor homology domains. The known ligands for Tie-2/TEK include angiopoietin (Ang)-1 and Ang-2. These RTKs play an important role in the development of the embryonic vasculature and continue to be expressed in adult endothelial cells. Tie-2/TEK is a novel RTK that is expressed almost exclusively by vascular endothelium. Expression of Tie-2/TEK is important for the development of the embryonic vasculature. Overexpression and/or mutation of Tie-2/TEK has been linked to pathogenic angiogenesis, and thus tumor growth, as well as myeloid leukemia.

Exemplary RTK-isoforms for targeting Tie/TEK-receptors include RTK isoforms such as Tie/TEK-IFPs set forth in SEQ ID NO: 22-25. Such RTK isoforms can be used for treatment of diseases and conditions in which the Tie/Tek receptor is implicated, including anti-angiogenesis therapy in diseases such as cancer, eye diseases, and rheumatoid arthritis. Other diseases and conditions that can be treated with TIE/TEK isoforms include inflammatory diseases such as arthritis, rheumatism, and psoriasis, benign tumors and preneoplastic conditions, myocardial angiogenesis, hemophilic joints, scleroderma, vascular adhesions, atherosclerotic plaque neovascularization, telangiectasia, and wound granulation. Additional targets for Tek receptor isoforms include diseases in which TEK is overexpressed, for example, chronic myeloid leukemia.

F. Methods of Producing CSR isoform Nucleic Acids and Polypeptides

Exemplary methods for generating CSR isoform nucleic acid molecules and polypeptides are provided herein. Such methods include in vitro synthesis methods for nucleic acid molecules such as PCR, synthetic gene construction and in vitro ligation of isolated and/or synthesized nucleic acid fragments. CSR isoform nucleic acid molecules also can be isolated by cloning methods, including PCR of RNA and DNA isolated from cells and screening of nucleic acid molecule libraries by hybridization and/or expression screening methods.

CSR isoform polypeptides can be generated from CSR isoform nucleic acid molecules using in vitro and in vivo synthesis methods. CSR isoforms can be expressed in any organism suitable to produce the required amounts and forms of isoform needed for administration and treatment. Expression hosts include prokaryotic and eukaryotic organisms such as E. coli, yeast, plants, insect cells, mammalian cells, including human cell lines and transgenic animals. CSR isoforms also can be isolated from cells and organisms in which they are expressed, including cells and organisms in which isoforms are produced recombinantly and those in which isoforms are synthesized without recombinant means such as genomically-encoded isoforms produced by alternative splicing events.

1. Synthetic Genes and Polypeptides

CSR isoform nucleic acid molecules and polypeptides can be synthesized by methods known to one of skill in the art using synthetic gene synthesis. In such methods, a polypeptide sequence of an CSR isoform is “back-translated” to generate one or more nucleic acid molecules encoding an isoform. The back-translated nucleic acid molecule is then synthesized as one or more DNA fragments such as by using automated DNA synthesis technology. The fragments are then operatively linked to form a nucleic acid molecule encoding an isoform. Nucleic acid molecules also can be joined with additional nucleic acid molecules such as vectors, regulatory sequences for regulating transcription and translation and other polypeptide-encoding nucleic acid molecules. Isoform-encoding nucleic acid molecules also can be joined with labels such as for tracking, including radiolabels, and fluorescent moieties.

The process of backtranslation uses the genetic code to obtain a nucleotide gene sequence for any polypeptide of interest, such as an CSR isoform. The genetic code is degenerate, 64 codons specify 20 amino acids and 3 stop codons. Such degeneracy permits flexibility in nucleic acid design and generation, allowing for example restriction sites to be added to facilitate the linking of nucleic acid fragments and the placement of unique identifier sequences within each synthesized fragment. Degeneracy of the genetic code also allows the design of nucleic acid molecules to avoid unwanted nucleotide sequences, including unwanted restriction sites, splicing donor or acceptor sites, or other nucleotide sequences potentially detrimental to efficient translation. Additionally, organisms sometimes favor particular codon usage and/or a defined ratio of GC to AT nucleotides. Thus, degeneracy of the genetic code permits design of nucleic acid molecules tailored for expression in particular organisms or groups of organisms. Additionally, nucleic acid molecules can be designed for different levels of expression based on optimizing (or non-optimizing) of the sequences. Back-translation is performed by selecting codons that encode a polypeptide. Such processes can be performed manually using a table of the genetic code and a polypeptide sequence. Alternatively, computer programs, including publicly available software can be used to generate back-translated nucleic acid sequences.

For example, an isoform such as the IFP set forth in SEQ ID NO:19 contains a sequence of 934 amino acids. The coding DNA sequence for this amino acid sequence (and in general of any other amino acid sequence) can be determined by a process of back-translation. A table for genetic code with no organism preference can be used. Alternatively, a genetic code table that incorporates codon preference for a particular organism, such as an expression host is selected. An exemplary nucleic acid sequence encoding SEQ ID NO:19 is set forth in SEQ ID NO: 26.

To synthesize a back-translated nucleic acid molecule, any method available in the art for nucleic acid synthesis can be used. For example, individual oligonucleotides corresponding to fragments of a CSR isoform-encoding sequence of nucleotides are synthesized by standard automated methods and mixed together in an annealing or hybridization reaction. Such oligonucleotides are synthesized and such annealing results in the self-assembly of the gene from the oligonucleotides using overlapping single-stranded overhangs formed upon duplexing complementary sequences, generally about 100 nucleotides in length. Single nucleotide “nicks” in the duplex DNA are sealed using ligation, for example with bacteriophage T4 DNA ligase. Restriction endonuclease linker sequences can for example, then be used to insert the synthetic gene into any one of a variety of recombinant DNA vectors suitable for protein expression. In another, similar method, a series of overlapping oligonucleotides are prepared by chemical oligonucleotide synthesis methods. Annealing of these oligonucleotides results in a gapped DNA structure. DNA synthesis catalyzed by enzymes such as DNA polymerase I can be used to fill in these gaps, and ligation is used to seal any nicks in the duplex structure. PCR and/or other DNA amplification techniques can be applied to amplify the formed linear DNA duplex.

Additional nucleotide sequences can be joined to a CSR isoform-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to an isoform-encoding nucleic acid molecule. Examples of such sequences include, but are not limited to, promoter sequences designed to facilitate intracellular protein expression, and secretion sequences designed to facilitate protein secretion. Additional nucleotide sequences such as sequences specifying protein binding regions also can be linked to isoform-encoding nucleic acid molecules. Such regions include, but are not limited to, sequences to facilitate uptake of an isoform into specific target cells, or otherwise enhance the pharmacokinetics of the synthetic gene.

CSR isoforms also can be synthesized using automated synthetic polypeptide synthesis. Cloned and/or in silico-generated polypeptide sequences can be synthesized in fragments and then chemically linked. Alternatively, isoforms can be synthesized as a single polypeptide. Such polypeptides can then be used in the assays and treatment administrations described herein.

2. Methods of Cloning and Isolating CSR Isoforms

CSR isoforms can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding an isoform, include for example, polymerase chain reaction (PCR) methods. A nucleic acid containing material can be used as a starting material from which an isoform-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify an isoform. For example, primers can be designed based on expressed sequences from which an isoform is generated. Primers can be designed based on back-translation of an isoform amino acid sequence. Nucleic acid molecules generated by amplification can be sequenced and confirmed to encode an isoform.

Nucleic acid molecules encoding isoforms also can be isolated using library screening. For example, a nucleic acid library representing expressed RNA transcripts as cDNAs can be screened by hybridization with nucleic acid molecules encoding CSR isoforms or portions thereof. For example, an intron sequence or portion thereof from a CSR gene can be used to screen for intron retention containing molecules based on hybridization to homologous sequences. Expression library screening can be used to isolate nucleic acid molecules encoding a CSR isoform. For example, an expression library can be screened with antibodies that recognize a specific isoform or a portion of an isoform. Antibodies can be obtained and/or prepared which specifically bind a CSR isoform or a region or peptide contained in an isoform. Antibodies which specifically bind an isoform can be used to screen an expression library containing nucleic acid molecules encoding an isoform, such as an IFP. Methods of preparing and isolating antibodies, including polyclonal and monoclonal antibodies and fragments therefrom are well-known in the art. Methods of preparing and isolating recombinant and synthetic antibodies also are well-known in the art. For example, such antibodies can be constructed using solid phase peptide synthesis or can be produced recombinantly, using nucleotide and amino acid sequence information of the antigen binding sites of antibodies that specifically bind a candidate polypeptide. Antibodies also can be obtained by screening combinatorial libraries containing variable heavy chains and variable light chains, or of antigen-binding portions thereof. Methods of preparing, isolating and using polyclonal, monoclonal and non-natural antibodies are reviewed, for example, in Kontermann and Dubel, eds. (2001) “Antibody Engineering” Springer Verlag; Howard and Bethell, eds. (2001) “Basic Methods in Antibody Production and Characterization” CRC Press; and O'Brien and Aitkin, eds. (2001) “Antibody Phage Display” Humana Press. Such antibodies also can be used to screen for the presence of an isoform polypeptide, for example, to detect the expression of a CSR isoform in a cell, tissue or extract.

3. Isoform Conjugates

CSR isoforms also can be provided as conjugates between the isoform and another agent. The conjugate can be used to target to a receptor with which the isoform interacts and/or to another targeted receptor for delivery of isoform. Such conjugates include linkage of a CSR isoform to a targeted agent and/or targeting agent. Conjugates can be produced by any suitable method including chemical conjugation or by expression of fusion proteins in which, for example, DNA encoding a targeted agent or targeting agent, with or without a linker region, is operatively linked to DNA encoding an RTK isoform. Conjugates also can be produced by chemical coupling, typically through disulfide bonds between cysteine residues present in or added to the components, or through amide bonds or other suitable bonds. Ionic or other linkages also are contemplated.

Pharmaceutical compositions can be prepared that CSR isoform conjugates and treatment effected by administering a therapeutically effective amount a conjugate, for example, in a physiologically acceptable excipient. CSR isoform conjugates also can be used in in vivo therapy methods such as by delivering a vector containing a nucleic acid encoding a CSR isoform conjugate as a fusion protein.

Conjugates can contain one or more CSR isoforms linked, either directly or via a linker, to one or more targeted agents: (CSR isoform)n, (L)q, and (targeted agent)m in which at least one CSR isoform is linked directly or via one or more linkers (L) to at least one targeted agent. Such conjugates also can be produced with any portion of a CSR isoform sufficient to bind a target, such as a target cell type for treatment. Any suitable association among the elements of the conjugate and any number of elements where n, and m are integer greater than 1 and q is zero or any integer greater then 1, is contemplated as long as the resulting conjugates interacts with a targeted CSR or targeted cell type.

In one example, a CSR isoform is used as a targeting agent to target another molecule (referred to herein as a targeted agent). For example, herstatin (SEQ ID NO:9) can be used as a targeting domain. In another example, an intron-encoded portion or domain is used as a targeting agent, for example ECDIIIa (see for example, U.S. Pat. No. 6,414,130 and U.S. Published Application No. 20040022785, incorporated by reference herein).

Examples of a targeted agent include drugs and other cytotoxic molecules such as toxins that act at or via the cell surface and those that act intracellularly. Examples of such moieties, include radionuclides, radioactive atoms that decay to deliver, e.g., ionizing alpha particles or beta particles, or X-rays or gamma rays, that can be targeted when coupled to a CSR isoform. Other examples include chemotherapeutics that can be targeted by coupling with an isoform. For example, geldanamycin targets proteosomes. An isoform-geldanamycin molecule can be directed to intracellular proteosomes, degrading the targeted isoform and liberating geldanamycin at the proteosome. Other toxic molecules include toxins, such as ricin, saporin and natural products from conches or other members of phylum mollusca. Another example of a conjugate with a targeted agent is a CSR isoform coupled, for example as a protein fusion, with an antibody or antibody fragment. For example, an isoform can be coupled to an Fc fragment of an antibody that binds to a specific cell surface marker to induce killer T cell activity in neutrophils, natural killer cells, and macrophages. A variety of toxins are well known to those of skil ll in the art.

Conjugates can contain one or more CSR isoforms linked, either directly or via a linker, to one or more targeting agents: (CSR isoform)n, (L)q, and (targeting agent)m in which at least one CSR isoform is linked directly or via one or more linkers (L) to at least one targeting agent. Any suitable association among the elements of the conjugate and any number of elements where n, and m are integer greater than 1 and q is zero or any integer greater then 1, is contemplated as long as the resulting conjugates interacts with a target, such as a targeted cell type.

Targeting agents include any molecule that targets a CSR isoform to a target such as a particular tissue or cell type or organ. Examples of targeting agents include cell surface antigens, cell surface receptors, proteins, lipids and carbohydrate moieties on the cell surface or within the cell membrane, molecules processed on the cell surface, secreted and other extracellular molecules. Molecules useful as targeting agents include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Exemplary molecules useful as targeting agents include ligands for receptors, such as proteinaceous and small molecule ligands, and antibodies and binding proteins, such as antigen-binding proteins.

Alternatively, the CSR isoform, which specifically interacts with a particular receptor (or receptors) is the targeting agent and it is linked to targeted agent, such as a toxin, drug or nucleic acid molecule. The nucleic acid molecule can be transcribed and/or translated in the targeted cell or it can be regulatory nucleic acid molecule.

The CSR and be linked directly to the targeted (or targeting agent) or via a linker. Linkers include peptide and non-peptide linkers and can be selected for functionalityh, such as to relieve or decrease stearic hindrance caused by proximity of a targeted agent or targeting agent to a CSR isoform and/or increase or alter other properties of the conjugate, such as the specificity, toxicity, solubility, serum stability and/or intracellular availability and/or to increase the flexibility of the linkage between a CSR isoform and a targeted agent or targeting agent. Examples of linkers and conjugation methods are known in the art (see, for example, WO 00/04926). CSRs also can be targeted using liposomes and other such moieties that direct delivery of encapsulated or entrapped molecules.

4. Expression Systems

CSR isoforms, including natural and combinatorial IFPs, can be produced by any means known in the art including in vivo and in vitro methods. CSR isoform can be expressed in any organism suitable to produce the required amounts and forms of CSR isoforms needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available for the expression of CSR isoforms. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

CSR isoforms also can be utilized or expressed as protein fusions. For example, an isoform fusion can be generated to add additional functionality to an isoform. Examples of isoform fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

a. Prokaryotic Expression

Prokaryotes, especially E. coli, provide a system for producing large amounts of proteins such as CSR isoforms. Transformation of E. coli is a simple and rapid technique well-known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Isoforms can be expressed in the cytoplasmic environment of E. coli. The cytoplasm is a reducing environment and for some molecules, this can result in the formation of insoluble inclusion bodies. Reducing agents such as dithiothreotol and P-mercaptoethanol and denaturants, such as guanidine-HCl and urea can be used to resolubilize the proteins. An alternative approach is the expression of CSR isoforms in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast

Yeasts such as Saccharomyces cerevisae, Schizosaccharomyces pombe, Yarrowia lipolytica, Kluyveromyces lactis and Pichia pastoris are useful expression hosts for production of CSR isoforms. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1. Expression vectors often include a selectable marker such as LEU2, TRP 1, HIS3 and URA3 for selection and maintenance of the transformed DNA. Proteins expressed in yeast are often soluble. Co-expression with chaperonins such as Bip and protein disulfide isomerase can improved expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as CSR isoforms. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express CSR isoforms. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ζ}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NSO (non-secreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be used to express CSR isoforms. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce CSR isoforms (see for example, Mayfield et al. (2003) PNAS 100:438-442). Because plants have different glycosylation patterns than mammalian cells, this can influence the choice of CSR isoforms produced in these hosts.

G. Biological Activity Assays

Generally, a CSR isoform is altered in one or more biological activities as compared to a wildtype or predominant form of a receptor. In vitro and in vivo assays can be used to monitor a biological activity of CSR isoforms. Exemplary in vitro and in vivo assays are provided herein for comparison of a biological activity of an RTK isoform to a biological activity of a wildtype or predominant form of an RTK. Many of the assays are applicable to other CSRs and CSR isoforms. In addition, numerous assays for biological activities of CSRs are known to one of skill in the art. Assays for RTK isoforms and RTKs include, but are not limited to, kinase assays, homodimerization and heterodimerization assays, protein:protein interaction assays, structural assays, cell signaling assays and in vivo phenotyping assays. Assays also include the use of animal models, including disease models in which a biological activity can be observed and/or measured. Dose response curves of an RTK isoform in such assays can be used to assess modulation of biological activities and as well as to determine therapeutically effective amounts of an RTK isoform for administration. Exemplary assays are described below.

1. Kinase Assays

Kinase activity can be detected and/or measured directly and indirectly. For example, antibodies against phosphotyrosine can be used to detect phosphorylation of an RTK, RTK isoform, an RTK:RTK isoform complex and phosphorylation of other proteins and signaling molecules. For example, activation of tyrosine kinase activity of an RTK can be measured in the presence of a ligand for an RTK. Transphosphorylation can be detected by anti-phosphotyrosine antibodies. Transphosphorylation can be measured and/or detected in the presence and absence of an RTK isoform, thus measuring the ability of an RTK isoform to modulate the transphosphorylation of an RTK. Briefly, cells expressing an RTK isoform or that have been exposed to an RTK isoform, are treated with ligand. Cells are lysed and protein extracts (whole cell extracts or fractionated extracts) are loaded onto a polyacrylamide gel, separated by electrophoresis and transferred to membrane, such as used for western blotting. Immunoprecipitation with anti-RTK antibodies also can be used to fractionate and isolate RTK proteins before performing gel electrophoresis and western blotting. The membranes can be probed with anti-phosphotyrosine antibodies to detect phosphorylation as well as probed with anti-RTK antibodies to detect total RTK protein. Control cells, such as cells not expressing RTK isoform and cells not exposed to ligand can be subjected to the same procedures for comparison.

Tyrosine phosphorylation also can be measured directly, such as by mass spectroscopy. For example, the effect of an RTK isoform on the phosphorylation state of an RTK can be measured, such as by treating intact cells with various concentrations of an RTK isoform and measuring the effect on activation of an RTK. The RTK can be isolated by immunoprecipitation and trypsinized to produce peptide fragments for analysis by mass spectroscopy. Peptide mass spectroscopy is a well-established method for quantitatively determining the extent of tyrosine phosphorylation for proteins; phosphorylation of tyrosine increases the mass of the peptide ion containing the phosphotyrosine, and this peptide is readily separated from the non-phosphorylated peptide by mass spectroscopy.

For example, tyrosine-1139 and tyrosine-1248 are known to be autophosphorylated in the ErbB-2 RTK. Trypsinized peptides can be empirically determined or predicted based on polypeptide sequence, for example by using ExPASy-PeptideMass program. The extent of phosphorylation of tyrosine-1139 and tyrosine-1248 can be determined from the mass spectroscopy data of peptides containing these tyrosines. Such assays can be used to assess the extent of auto-phosphorylation of an RTK isoform and the ability of an RTK isoform to transphosphorylate and RTK.

2. Complexation

Complexation, such as dimerization of RTKs and RTK isoforms can be detected and/or measured. For example, isolated polypeptides can be mixed together, subject to gel electrophoresis and western blotting. RTKs and/or RTK isoforms also can be added to cells and cell extracts, such as whole cell or fractionated extracts, can be subject to gel electrophoresis and western blotting. Antibodies recognizing the polypeptides can be used to detect the presence of monomers, dimers and other complexed forms. Alternatively, labeled RTKs and/or labeled RTK isoforms can be detected in the assays. Such assays can be used to compare homodimerization of an RTK or heterodimerization of two or more RTKs in the presence and absence of an RTK isoform. Assays also can be performed to assess homodimerization of an RTK isoform and/or its ability to heterodimerize with an RTK. For example an ErbB-2 RTK isoform can be assessed for its ability to heterodimerize with ErbB-2, ErbB-3 and ErbB-4. Additionally, an ErbB-2 RTK isoform can be assessed for its ability to modulate the ability of ErbB-2 to homodimerize with itself.

3. Ligand Binding

Generally, RTKs bind one or more ligands. Ligand binding modulates the activity of the receptor and thus modulates, for example, signaling within a signal transduction pathway. Ligand binding of an RTK isoform and ligand binding of an RTK in the presence of an RTK isoform can be measured. For example, labeled ligand such as radiolabeled ligand can be added to purified or partially purified RTK in the presence and absence (control) of an RTK isoform. Immunoprecipitation and measurement of radioactivity can be used to quantify the amount of ligand bound to an RTK in the presence and absence of an RTK isoform. An RTK isoform also can be assessed for ligand binding such as by incubating an RTK isoform with labeled ligand and determining the amount of labeled ligand bound by an RTK isoform, for example, as compared to an amount bound by a wildtype or predominant form of a corresponding RTK.

4. Cell Proliferation Assays

A number of RTKs, for example VEGFR, are involved in cell proliferation. Effects of an RTK isoform on cell proliferation can be measured. For example, ligand can be added to cells expressing an RTK. An RTK isoform can be added to such cells before, concurrently or after ligand addition and effects on cell proliferation measured. Alternatively an RTK isoform can be expressed in such cell models, for example using an adenovirus vector. For example, a VEGFR isoform is added to endothelial cells expressing VEGFR. Following isoform addition, VEGF ligand is added and the cells are incubated at standard growth temperature (e.g. 37° C.) for several days. Cells are trypsinized, stained with trypan blue and viable cells are counted. Cells not exposed to VEGFR isoform and/or ligand are used as controls for comparison.

5. Cell Disease Model Assays

Cells from a disease or condition or which can be modulated to mimic a disease or condition can be used to measure/and or detect the effect of an CSR isoform. An RTK isoform is added or expressed in cells and a phenotype is measured or detected in comparison to cells not exposed to or not expressing an RTK isoform. Such assays can be used to measure effects including effects on cell proliferation, metastasis, inflammation, angiogenesis, pathogen infection and bone resorption.

For example, effects of a MET isoform can be measured using such assays. A liver cell model such as HepG2 liver cells can be used to monitor the infectivity of malaria in culture by sporozoites. An RTK isoform such as a MET isoform can be added to the cells and/or expressed in the cells. Infection of such cells with malaria sporozoites is then measured, such as by staining and counting the EEFs (exoerythrocytic forms) of the sporozoite that are produced as a result of infection Carrolo et al. (2003) Nat Med 9(11):1363-1369. Effects of an RTK isoform can be assessed by comparing results to cells not exposed or expressing an RTK isoform and/or uninfected cells.

Effects of an RTK isoform also can be measured in angiogenesis. For example, tubule formation by endothelial cells such as human umbilical vein endothelial cells (HUVEC) in vitro can be used as an assay to measure angiogenesis and effects on angiogenesis. Addition of varying amounts of an RTK isoform to an in vitro angiogenesis assay is a method suitable for screening the effectiveness of an RTK isoform as a modulator of angiogenesis.

Bone resorption can be measured in cell culture to measure effectiveness of an RTK-isoform, such as by using osteoclast cultures. Osteoclasts are highly differentiated cells of hematopoietic origin that resorb bone in the organism, and are able to resorb bone from bone slices in vitro. Methods for cell culture of osteoclasts and quantitative techniques for measuring bone resorption in osteoclast cell culture have been described in the art. For example, mononuclear cells can be isolated from human peripheral blood and cultured. Addition and/or expression of an RTK isoform can be used to assess effects on osteoclast formation such as by measuring multinucleated cells positive for tartrate-resistant acid phosphatase and resorbed area and collagen fragments released from bone slices. Dose response curves can be used to determine therapeutically effective amounts of an RTK isoform necessary to modulate bone resorption.

6. Animal Models

Animal models can be used to assess the effect of an RTK isoform. For example, RTK isoform effects on cancer cell proliferation, migration and invasiveness can be measured. In one such assay, cancer cells such as ovarian cancer cells are infected with an adenovirus expressing an RTK isoform. After a culturing period in vitro, cells are trypsinized, suspended in a suitable buffer and injected into mice (e.g., into flanks and shoulders of model mice such as Balb/c nude mice). Tumor growth is monitored over time. Control cells, not expressing an RTK-isoform, can be injected into mice for comparison. Similar assays can be performed with other cell types and animal models, for example, murine lung carcinoma (LLC) cells and C57BL/6 mice and SCID mice. Effects of RTK isoforms on ocular disorders can be assessed using assays such as a corneal micropocket assay. Briefly, mice receive cells expressing an RTK isoform (or control) by injection 2-3 days before the assay. Subsequently, the mice are anesthetized, and pellets of a ligand such as VEGF are implanted into the corneal micropocket of the eyes. Neovascularization is then measured, for example, 5 days following implantation. The effect of an RTK-isoform on angiogenesis as compared to a control is then assessed. Any animal models known in the art can be used to assess the effect of a CSR isoform such as an RTK isoform, including transgenic mice, such as humanized transgenic mouse models such as atherosclerosis mice expressing DR and DQ major histocompatibility complex II molecules, which can be used as a model for example, for autoimmune diseases, including rheumatoid arthritis, celiac disease, multiple sclerosis, and insulin-dependent diabetes mellitus (Gregersen et al. (2004) Tissue Antigens 63(5):383-94), Apolipoprotein-E deficient mice (ApoE^{-/-}), which can be used as a model for atherosclerosis, IL-10 knockout mice, which can be used as a model, for example, for inflammatory bowel disease and Chrohn's disease (Scheinin et al. (2003) Clin. Exp. Immunol. 133(1):38-43), and Alzheimer's disease models such as transgenic mice overexpressing mutant amyloid precursor protein and mice expressing familial autosomal dominant-linked PS 1. Animal models also include animals induced or treated to exhbit disease such as EAE induced animals used as a model for multiple sclerosis.

H. Preparation, Formulation and Administration of CSR Isoforms and CSR Isoform Compositions

CSR isoforms and CSR isoform compositions, including RTK isoforms and RTK isoform compositions, can formulated for administration by any route known to those of skill in the art including intramuscular, intravenous, intradermal, intraperitoneal injection, subcutaneous, epidural, nasal, oral, rectal, topical, inhalational, buccal (e.g., sublingual), and transdermal administration or any route. CSR isoforms can be administered by any convenient route, for example by infusion or bolus injection, by absorption through epithelial or mucocutaneous linings (e.g., oral mucosa, rectal and intestinal mucosa, etc.) and can be administered with other biologically active agents, either sequentially, intermittently or in the same composition. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Administration also can include controlled release systems including controlled release formulations and device controlled release, such as by means of a pump. The most suitable route in any given case will depend on the nature and severity of the disease or condition being treated and on the nature of the particular composition which is used.

Various delivery systems are known and can be used to administer CSR isoforms, such as but not limited to, encapsulation in liposomes, microparticles, microcapsules, recombinant cells capable of expressing the compound, receptor mediated endocytosis, and delivery of nucleic acid molecules encoding CSR isoforms such as retrovirus delivery systems.

Pharmaceutical compositions containing CSR isoforms can be prepared. Generally, pharmaceutically acceptable compositions are prepared in view of approvals for a regulatory agency or otherwise prepared in accordance with generally recognized pharmacopoeia for use in animals and in humans. Pharmaceutical compositions can include carriers such as a diluent, adjuvant, excipient, or vehicle with which an isoform is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. The formulation should suit the mode of administration.

Formulations are provided for administration to humans and animals in unit dosage forms, such as tablets, capsules, pills, powders, granules, sterile parenteral solutions or suspensions, and oral solutions or suspensions, and oil:water emulsions containing suitable quantities of the compounds or pharmaceutically acceptable derivatives thereof. Pharmaceutically therapeutically active compounds and derivatives thereof are typically formulated and administered in unit dosage forms or multiple dosage forms. Unit dose forms as used herein refer to physically discrete units suitable for human and animal subjects and packaged individually as is known in the art. Each unit dose contains a predetermined quantity of a therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging.

Dosage forms or compositions containing active ingredient in the range of 0.005% to 100% with the balance made up from non toxic carrier can be prepared. For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be provided as unit dose suppositories. These can be prepared by admixing the active compound with one or more conventional solid carriers, for example, cocoa butter, and then shaping the resulting mixture.

Formulations suitable for topical application to the skin or to the eye include ointments, creams, lotions, pastes, gels, sprays, aerosols and oils. Exemplary carriers include vaseline, lanoline, polyethylene glycols, alcohols, and combinations of two or more thereof. The topical formulations also can contain 0.05 to 15, 20, 25 percent by weight of thickeners selected from among hydroxypropyl methyl cellulose, methyl cellulose, polyvinylpyrrolidone, polyvinyl alcohol, poly (alkylene glycols), polyhydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. A topical formulation in the liquid state can be also present in a hydrophilic three-dimensional polymer matrix in the form of a strip or contact lens, from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions of CSR isoforms can be formulated for parenteral administration by injection, e.g., by bolus injection or continuous infusion. Formulations for injection can be presented in unit dosage form, e.g., in ampules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration can be presented as discrete patches adapted to remain in intimate contact with the epidermis of the recipient for a prolonged period of time. Such patches suitably contain the active compound as an optionally buffered aqueous solution of, for example, 0.1 to 0.2 M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

Pharmaceutical compositions also can be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

In certain embodiments, liposomes and/or nanoparticles also can be employed with CSR isoform administration. Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized about 0.1 micometers in diameber) can be designed using polymers that can be degraded in vivo. Biodegradable polyalkyl-cyanoacrylate nanoparticles that meet these requirements are contemplated for use herein, and such particles can be easily made.

Administration methods can be employed to decrease the exposure of CSR isoforms to degradative processes, such as proteolytic degradation and immunological intervention via antigenic and immunogenic responses. Examples of such methods include local administration at the site of treatment. CSR isoforms also can be modified to modulate serum stability and half-life as well as reduce immunogenicity. Such modifications can be effected by any means known in the art and include addition of molecules to CSR isoforms such as pegylation, and addition of serum albumin, IgG, and glycosylation (Raju et al. (2001) Biochemistry 40(3):8868-76; van Der Auwera et al. (2001) Am J Hematol. 66(4):245-51.).

Pegylation of therapeutics has been reported to increase resistance to proteolysis; increase plasma half-life, and decrease antigenicity and immunogencity. Examples of pegylation methodologies are known in the art (see for example, Lu and Felix, Int. J. Peptide Protein Res., 43: 127-138, 1994; Lu and Felix, Peptide Res., 6: 142-6, 1993; Felix et al., Int. J. Peptide Res., 46: 253-64, 1995; Benhar et al., J. Biol. Chem., 269: 13398-404, 1994; Brumeanu et al., J Immunol., 154: 3088-95, 1995; see also, Caliceti et al. (2003) Adv. Drug Deliv. Rev. 55(10):1261-77 and Molineux (2003) Pharmacotherapy 23 (8 Pt 2):3S-8S). Pegylation also can be used in the delivery of nucleic acid molecules in vivo. For example, pegylation of adenovirus can increase stability and gene transfer (see, e.g., Cheng et al. (2003) Pharm. Res. 20(9): 1444-51).

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects), administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

A CSR isoform is included in the pharmaceutically acceptable carrier in an amount sufficient to exert a therapeutically useful effect in the absence of undesirable side effects on the patient treated. Therapeutically effective concentration can be determined empirically by testing the compounds in known in vitro and in vivo systems, such as the assays provided herein.

The concentration of a CSR isoform in the composition will depend on absorption, inactivation and excretion rates of the complex, the physicochemical characteristics of the complex, the dosage schedule, and amount administered as well as other factors known to those of skill in the art.

The amount of a CSR isoform to be administered for the treatment of a disease or condition, for example cancer, autoimmune disease and infection can be determined by standard clinical techniques. In addition, in vitro assays and animal models can be employed to help identify optimal dosage ranges. The precise dosage, which can be determined empirically, can depend on the route of administration and the seriousness of the disease. Suitable dosage ranges for administration can range from about 0.01 pg/kg body weight to 1 mg/kg body weight and more typically 0.05 mg/kg to 200 mg/kg CSR isoform: patient weight.

A CSR isoform can be administered at once, or can be divided into a number of smaller doses to be administered at intervals of time. CSR isoforms can be administered in one or more doses over the course of a treatment time for example over several hours, days, weeks, or months. In some cases, continuous administration is useful. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them.

I. In Vivo Expression of CSR Isoforms

CSR isoforms can be administered as nucleic acid molecules encoding a CSR isoform, including ex vivo techniques and direct in vivo expression. Methods for administering CSR isoforms include viral vector administration, administration of nucleic acids ex vivo and in vivo and transfer of nucleic acids to endogenous chromosomes. For ex vivo treatment, a patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). Techniques suitable for the transfer of nucleic acid into mammalian cells in vitro include the use of liposomes and cationic lipids (e.g., DOTMA, DOPE and DC-Chol) electroporation, microinjection, cell fusion, DEAE-dextran, and calcium phosphate precipitation methods. Methods of DNA delivery can be used to express CSR isoforms in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

Cells into which a nucleic acid can be introduced for purposes of therapy encompass any desired, available cell type appropriate for the disease or condition to be treated, including but not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof. Tumor cells also can be target cells for in vivo expression of CSR isoforms. Cells used for in vivo expression of an isoform also include cells autologous to the patient. Such cells can be removed from a patient, nucleic acids for expression of a CSR isoform introduced, and then administered to a patient such as by injection or engraftment.

A CSR isoform can be expressed by a virus and administered to a subject in need of treatment. Virus vectors suitable for gene therapy include adenovirus, adeno-associated virus, retroviruses, lentiviruses Adenovirus expression technology is well-known in the art and adenovirus production and administration methods also are well known. Adenovirus serotypes are available, for example, from the American Type Culture Collection (ATCC, Rockville, Md.). Adenovirus can be used ex vivo, for example, cells are isolated from a patient in need of treatment, and transduced with a CSR isoform-expressing adenovirus vector. After a suitable culturing period, the transduced cells are administered to a subject, locally and/or systemically. Alternatively, CSR isoform-expressing adenovirus particles are isolated and formulated in a pharmaceutically-acceptable carrier for delivery of a therapeutically effective amount to prevent, treat or ameliorate a disease or condition of a subject. Typically, adenovirus particles are delivered at a dose ranging from 1 particle to 10¹⁴ particles per kilogram subject weight, generally between 10⁶ or 10⁸ particles to 10¹² particles per kilogram subject weight. In some situations it is desirable to provide a nucleic acid source with an agent that targets cells, such as an antibody specific for a cell surface membrane protein or a target cell, or a ligand for a receptor on a target cell. Where liposomes are employed, proteins which bind to a cell surface membrane protein associated with endocytosis can be used for targeting and/or to facilitate uptake, e.g. capsid proteins or fragments thereof tropic for a particular cell type, antibodies for proteins which undergo internalization in cycling, and proteins that target intracellular localization and enhance intracellular half-life.

CSR isoforms also can be used in ex vivo gene expression therapy using non-viral vectors. For example, cells can be engineered which express a CSR isoform, such as by integrating a CSR isoform sequence into a genomic location, either operatively linked to regulatory sequences or such that it is placed operatively linked to regulatory sequences in a genomic location. Such cells then can be administered locally or systemically to a subject, such as a patient in need of treatment.

In vivo expression of a CSR isoform can be linked to expression of additional molecules. For example, expression of a CSR isoform can be linked with expression of a cytotoxic product such as in an engineered virus or expressed in a cytotoxic virus. Such viruses can be targeted to a particular cell type that is a target for a therapeutic effect. The expressed CSR isoform can be used to enhance the cytotoxicity of the virus.

In vivo expression of a CSR isoform can include operatively linking a CSR isoform encoding nucleic acid molecule to specific regulatory sequences such as a cell-specific or tissue-specific promoter. CSR isoforms also can be expressed from vectors that specifically infect and/or replicate in target cell types and/or tissues. Inducible promoters can be used to selectively regulate CSR isoform expression.

J. Exemplary Treatments and Studies with CSR Isoforms

Provided herein are methods of treatment with CSR isoforms for diseases and conditions. CSR isoforms such as RTK isoforms can be used in the treatment of a variety of diseases and conditions, including those described herein. Treatment can be effected by administering by suitable route formulations of the polyeptides, which can be provided in compositions as polypeptides and can be linked to targeting agents, for targeted delivery or encapsulated in delivery vehicles, such as liposomes. Alternatively, nucleic acids encoding the polypeptides can be administered as naked nucleic acids or in vectors, particularly gene therapy vectors. Such gene therapy can be effected ex vivo by removing cells from a subject, introducing the vector or nucleic acid into the cells and then reintroducing the modified cells. Gene therapy also can be effect in vivo by directly administering the nucleic acid or vector.

Treatments using the CSR isoforms provided herein, include, but are not limited to treatment of angiogenesis-related diseases and conditions including ocular diseases, atherosclerosis, cancer and vascular injuries, neurodegenerative diseases, including Alzheimer's disease, inflammatory diseases and conditions, including atherosclerosis, diseases and conditions associated with cell proliferation including cancers, and smooth muscle cell-associated conditions, and various autoimmune diseases. Exemplary treatments and preclinical studies are described for treatments and therapies with RTK isoforms. Such descriptions are meant to be exemplary only and are not limited to a particular RTK isoform. One of skill in the art can assess based on the type of disease to be treated, the severity and course of the disease, whether the molecule is administered for preventive or therapeutic purposes, previous therapy, the patient's clinical history and response to therapy, and the discretion of the attending physician appropriate dosage of a molecule to administer.

1. Angiogenesis-Related Ocular Conditions

RTK isoforms including, but not limited to, VEGFR, PDGFR, TIE/TEK, FGF, EGFR, and EphA can be used in treatment of angiogenesis related ocular diseases and conditions, including ocular diseases involving neovascularization. Ocular neovascular disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage also is associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Karposi sarcoma, Mooren ulcer, Terrien's marginal degeneration, marginal keratolysis, rheumatoid arthritis, systemic lupus, polyarteritis, trauma, Wegener's sarcoidosis, Scleritis, Stevens Johnson disease, pemphigoid radial keratotomy, and corneal graph rejection. Diseases associated with retinal/choroidal neovascularization include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargart's disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

RTK isoform therapeutic effects on angiogenesis such as in treatment of ocular diseases can be assessed in animal models, for example in cornea implants, such as described herein. For example, modulation of angiogenesis such as for an RTK can be assessed in a nude mouse model such as epidermoid A431 tumors in nude mice and VEGF-or PIGF-transduced rat C6 gliomas implanted in nude mice. CSR isoforms can be injected as protein locally or systemically, Alternatively cells expressing CSR isoforms can be inoculated locally or at a site remote to the tumor. Tumors can be compared between control treated and CSR isoform treated models to observe phenotypes of tumor inhibition including poorly vascularized and pale tumors, necrosis, reduced proliferation and increased tumor-cell apoptosis. In one such treatment, Flt-1 isoforms are used to treat ocular disease and assessed in scuh models.

Examples of ocular disorders that can be treated with TIE/TEK isoforms are eye diseases characterized by ocular neovascularization including, but not limited to, diabetic retinopathy (a major complication of diabetes), retinopathy of prematurity (this devastating eye condition, that frequently leads to chronic vision problems and carries a high risk of blindness, is a severe complication during the care of premature infants), neovascular glaucoma, retinoblastoma, retrolental fibroplasia, rubeosis, uveitis, macular degeneration, and corneal graft neovascularization. Other eye inflammatory diseases, ocular tumors, and diseases associated with choroidal or iris neovascularization also can be treated with TIE/TEK isoforms.

PDGFR isoforms also can be used in the treatment of proliferative vitreoretinopathy. For example, an expression vector such as a retroviral vector is constructed containing a nucleic acid molecule encoding a PDGFR isoform. Rabbit conjunctival fibroblasts (RCFs) are produced which contain the expression vector by transfection, such as for a retrovirus vector, or by transformation, such as for a plasmid or chromosomal based vector. Expression of PDGFR isoform can be monitored in cells by means known in the art including use of an antibody which recognizes PDGFR isoform and by use of a peptide tag (e.g. a myc tag) and corresponding antibody. RCFs are injected into the vitreous part of an eye. For example, in a rabbit animal model, approximately 1×10⁵ RCFs are injected by gas vitreomy. Retrovirus expressing PDGFR isoform, ˜2×10⁷ CFU is injected on the same day. Effects on proliferative vitreoretinopathy can be observed, for example, 2-4 weeks following surgery, such as attenuation of the disease symptoms.

EphA isoforms can be used to treat diseases or conditions with misregulated and/or inappropriate angiogenesis, such as in eye diseases. For example, an EphA isoform can be assessed in an animal model such as a mouse corneal model for effects on ephrinA-1 induced angiogenesis. Hydron pellets containing ephrina-1 alone or with EphA isoform protein are implanted in mouse cornea. Visual observations are taken on days following implantation to observe EphA isoform inhibition or reduction of angiogenesis. Anti-angiogenic treatments and methods such as described for VEGFR isoforms are applicable to EphA isoforms.

2. Angiogenesis Related Atherosclerosis

RTK isoforms, for example VEGFR Flt-1 and TIE/TEK isoforms, can be used to treat angiogenesis conditions related to atherosclerosis such as neovascularization of atherosclerosis plaques. Plaques formed within the lumen of blood vessels have been shown to have angiogenic stimulatory activity. VEGF expression in human coronary atherosclerotic lesions is associated with the progression of human coronary atherosclerosis.

Animal models can be used to assess RTK isoforms in treatment of atherosclerosis. Apolipoprotein-E deficient mice (ApoE^{-/-}) are prone to atherosclerosis. Such mice are treated by injecting an RTK isoform, for example a VEGFR isoform, such as a Flt-1 IFP protein over a time course such as for 5 weeks starting at 5, 10 and 20 weeks of age. Lesions at the aortic root are assessed between control ApoE^{-/-} mice and isoform-treated ApoE^{-/-} mice to observe reduction of atherosclerotic lesions in isoform-treated mice.

3. Additional Angiogenesis-Related Treatments

RTK isoforms such as VEGFR isoforms, for example, Flt1 isoforms, and EphA isoforms also can be used to treat angiogenic and inflammatory-related conditions such as proliferation of synoviocytes, infiltration of inflammatory cells, cartilage destruction and pannus formation, such as are present in rheumatoid arthritis (RA). An autoimmune model of collagen type-II induced arthritis, such as polyarticular arthritis induced in mice, can be used as a model for human RA. Mice treated with an RTKisoform, such as by local injection of protein, can be observed for reduction of arthritic symptoms including paw swelling, erythema and ankylosis. Reduction in synovial angiogenesis and synovial inflammation also can be observed. Angiogenesis plays a key role in the formation and maintainance of the pannus in RA. RTK isoforms can be used alone and in combination with other isoforms and other treatments to modulate angiogenesis. For example, angiogenesis inhbiotrs can be used in combination with RTK isoforms to treat RA. Exemplary angiogenesis inhibitors include, but are not limited to, angiostatin, antangiogenic antithrombin III, canstatin, cartilage derived inhibitor, fibronectin fragement, IL-12, vasculostatin and others known in the art (see for example, Paleolog (2002) Arthritis Research Therapy 4 (supp 3) S81-S90)

Other angiogenesis-related conditions amenable to treatment with VEGFR isoforms include hemangioma. One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Many cases of hemangiomas exist that cannot be treated or are difficult to treat with therapeutics currently in use.

VEGFR isoforms can be employed in the treatment of such diseases and conditions where angiogenesis is responsible for damage such as in Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula. Diseases and disorders characterized by undesirable vascular permeability also can be treated by VEGFR isoforms. These include edema associated with brain tumors, ascites associated with malignancies, Meigs' syndrome, lung inflammation, nephrotic syndrome, pericardial effusion and pleural effusion.

Angiogenesis also is involved in normal physiological processes such as reproduction and wound healing. Angiogenesis is an important step in ovulation and also in implantation of the blastula after fertilization. Modulation of angiogenesis by VEGFR isoforms can be used to induce amenorrhea, to block ovulation or to prevent implantation by the blastula. VEGFR isoforms also can be used in surgical procedures. For example, in wound healing, excessive repair or fibroplasia can be a detrimental side effect of surgical procedures and can be caused or exacerbated by angiogenesis. Adhesions are a frequent complication of surgery and lead to problems such as small bowel obstruction.

PDGFR isoforms can be used in the regulation of neointima formation after arterial injury such as in arterial surgery. For example PDGFRB isoforms can be used to regulate PDGF-BB induced cell proliferation such as involved in neointima formation. PDGFR isoforms can be assessed for example, in a balloon-injured rooster femoral artery model. An adenovirus vector expressing a PDGFR isoform is constructed and transduced in vivo in the arterial model. Neointima-associated thrombosis is assessed in the transduced arteries to observe reduction as compared with controls.

RTK isoforms useful in treatment of angiogenesis-related diseases and conditions also can be used in combination therapies such as with anti-angiogenesis drugs, molecules which interact with other signaling molecules in RTK-related pathways, including modulation of VEGFR ligands, For example, the known anti-rheumatic drug, bucillamine (BUC), was shown to include within its mechanism of action the inhibition of VEGF production by synovial cells. Anti-rheumatic effects of BUC are mediated by suppression of angiogenesis and synovial proliferation in the arthritic synovium through the inhibition of VEGF production by synovial cells. Combination therapy of such drugs with VEGFR isoforms can allow multiple mechanisms and sites of action for treatment.

4. Cancers

RTK isoforms such as isoforms of EGFR, TIE/TEK, VEGFR, MET and FGFR can be used in treatment of cancers. RTK isoforms including, but not limited to, EGFR RTK isoforms, such as ErbB2 and ErbB3 isoforms, VEGFR isoforms such as Flt1 isoforms, FGFR isoforms such as FGFR4 isoforms, and EphA1 isoforms can be used to treat cancer. Examples of cancer to be treated herein include, but are not limited to, carcinoma, lymphoma, blastoma, sarcoma, and leukemia or lymphoid malignancies. Additional examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Combination therapies can be used with EGFR isoforms including anti-hormonal compounds, cardioprotectants, and anti-cancer agents such as chemotherapeutics and growth inhibitory agents.

Cancers treatable with EGFR isoforms are generally cancers expressing an EGFR receptor. Such cancers can be identified by any means known in the art for detecting EGFR expression. An example of an ErbB2 expression diagnostic/prognostic assay available includes HERCEPTEST.RTM. (Dako). Paraffin embedded tissue sections from a tumor biopsy are subjected to the IHC assay and accorded a ErbB2 protein staining intensity criteria. Tumors accorded with less than a threshold score can be characterized as not overexpressing ErbB2, whereas those tumors with greater than or equal to a threshold score can be characterized as overexpressing ErbB2. In one example of treatment, erbB2-overexpressing tumors are assessed as candidates for treatment with an EGFR isoform such as an erbB2 isoform.

TIE/TEK isoforms can be used in the treatment of cancers such as by modulating tumor-related angiogenesis. Vascularization is involved in regulating cancer growth and spread. For example, inhibition of angiogenesis and neovascularization inhibits solid tumor growth and expansion. Tie/Tek receptors such as Tie2 have been shown to influence vascular development in normal and cancerous tissues. TIE/TEK isoforms can be used as an inhibitor of tumor angiogenesis. A TIE/TEK isoform is produced such as by expression of the protein in cells. For example, secreted forms of TIE/TEK isoform can be expressed in cells and harvested from the media. Protein can be purified or partially-purified by biochemical means known in the art and by uses of antibody purification, such as antibodies raised against TIE/TEK isoform or a portion thereof or by use of a tagged TIE/TEK isoform and a corresponding antibody. Effects on angiogenesis can be monitored in an animal model such as by treating rat cornea with TIE/TEK isoform formulated as conditioned media in hydron pellets surgically implanted into a micropocket of a rat cornea or as purified protein (e.g. 100 μg/dose) administered to the window chamber. For example, rat models such as F344 rats with avascular corneas can be used in combination with tumor-cell conditioned media or by implanting a fragment of a tumor into the window chamber of an eye to induce angiogenesis. Corneas can be examined histologically to detect inhibition of angiogenesis induced by tumor-cell conditioned media. TIE/TEK isoforms also can be used to treat malignant and metastatic conditions such as solid tumors, including primary and metastatic sarcomas and carcinomas.

FGFR4 isoforms can be used to treat cancers, for example pituitary tumors. Animal models can be used to mimic progression of human pituitary tumor progress. For example, an N-terminally shortened form of FGFR, ptd-FGFR4, expressed in transgenic mice recapitulates pituitary tumorigenesis (Ezzat et al. (2002) J. Clin. Invest. 109:69-78), including pituitary adenoma formation in the absence of prolonged and massive hyperplasia. FGFR4 isoforms can be administered to ptd-FGFR4 mice and the pituitary architecture and course of tumor progression compared with control mice.

5. Alzheimer's Disease

EGFR isoforms also can be used to treat Alzheimer's disease and related conditions. A variety of mouse models are available for human Alzheimer's disease including transgenic mice overexpressing mutant amyloid precursor protein and mice expressing familial autosomal dominant-linked PSI and mice expressing both proteins (PS1 M146L/APPK670N:M671L). Alzheimer's models are treated such as by injection of ErbB isoforms. Plaque development can be assessed such as by observation of neuritic plaques in the hippocampus, entorhinal cortex, and cerebral cortex. using staining and antibody immunoreactivity assays.

6. Smooth Muscle Proliferative-Related Diseases and Conditions

EGFR isoforms including ErbB isoforms can be utilized for the treatment of a variety of diseases and conditions involving smooth muscle cell proliferation in a mammal, such as a human. An example is treatment of cardiac diseases involving proliferation of vascular smooth muscle cells (VSMC) and leading to intimal hyperplasia such as vascular stenosis, restenosis resulting from angioplasty or surgery or stent implants, atherosclerosis and hypertension. In such conditions, an interplay of various cells and cytokines released act in autocrine, paracrine or juxtacrine manner, which result in migration of VSMCs from their normal location in media to the damaged intima. The migrated VSMCs proliferate excessively and lead to thickening of intima, which results in stenosis or occlusion of blood vessels. The problem is compounded by platelet aggregation and deposition at the site of lesion. α-thrombin, a multifunctional serine protease, is concentrated at site of vascular injury and stimulates VSMCs proliferation. Following activation of this receptor, VSMCs produce and secrete various autocrine growth factors, including PDGF-AA, HB-EGF and TGF. EGFRs are involved in signal transduction cascades that ultimately result in migration and proliferation of fibroblasts and VSMCs, as well as stimulation of VSMCs to secrete various factors that are mitogenic for endothelial cells and induction of chemotactic response in endothelial cells. Treatment with EGFR isoforms can be used to modulate such signaling and responses.

EGFR isoforms such as ErbB2 and ErbB3 isoforms can be used to treat conditions where EGFRs such as ErbB2 and ErbB3 modulate bladder SMCs, such as bladder wall thickening that occurs in response to obstructive syndromes affecting the lower urinary tract. EGFR isoforms can be used in controlling proliferation of bladder smooth muscle cells, and consequently in the prevention or treatment of urinary obstructive syndromes.

EGFR isoforms can be used to treat obstructive airway diseases with underlying pathology involving smooth muscle cell proliferation. One example is asthma which manifests in airway inflammation and bronchoconstriction. EGF has been shown to stimulate proliferation of human airway SMCs and is likely to be one of the factors involved in the pathological proliferation of airway SMCs in obstructive airway diseases. EGFR isoforms can be used to modulate effects and responses to EGF by EGFRs.

7. Combination Therapies

CSR isoforms such as RTK isoforms can be used in combination with each other and with other existing drugs and therapeutics to treat diseases and conditions. For example, as described herein a number of RTK-isoforms can be used to treat angiogenesis-related conditions and diseases and/or control tumor proliferation. Such treatments can be performed in conjunction with anti-angiogenic and/or anti-tumorigenic drugs and/or therapeutics. Examples of anti-angiogenic and antitumorigenic drugs and therapies useful for combination therapies include tyrosine kinase inhibitors and molecules capable of modulating tyrosine kinase signal transduction can be used in combination therapies including, but not limited to, 4-aminopyrrolo[2,3-d]pyrimidines (see for example, U.S. Pat. No. 5,639,757), and quinazoline compounds and compositions (e.g., U.S. Pat. No. 5,792,771. Other compounds useful in combination therapies include steroids such as the angiostatic 4,9(11)-steroids and C21-oxygenated steroids, angiostatin, endostatin, vasculostatin, canstatin and maspin, angiopoietins, bacterial polysaccharide CM101 and the antibody LM609 (U.S. Pat. No. 5,753,230), thrombospondin (TSP-1), platelet factor 4 (PF4), interferons, metalloproteinase inhibitors, pharmacological agents including AGM-1470/TNP-470, thalidomide, and carboxyamidotriazole (CAI), cortisone such as in the presence of heparin or heparin fragments, anti-Invasive Factor, retinoic acids and paclitaxel (U.S. Pat. No. 5,716,981; incorporated herein by reference), shark cartilage extract, anionic polyamide or polyurea oligomers, oxindole derivatives, estradiol derivatives and thiazolopyrimidine derivatives.

Treatment of cancers including treatment of cancers overexpressing an EGFR can include combination therapy with an anticancer agent, a chemotherapeutic agent and growth inhibitory agent, including coadministration of cocktails of different chemotherapeutic agents. Examples of chemotherapeutic agents include taxanes (such as paclitaxel and doxetaxel) and anthracycline antibiotics. Preparation and dosing schedules for such chemotherapeutic agents can be used according to manufacturers' instructions or as determined empirically by the skilled practitioner. Preparation and dosing schedules for such chemotherapy also are described in Chemotherapy Service Ed., M. C. Perry, Williams & Wilkins, Baltimore, Md. (1992).

Additional compounds can be used in combination therapy with RTK isoforms. Anti-hormonal compounds can be used in combination therapies, such as with EGFR isoforms. Examples of such compounds include an anti-estrogen compound such as tamoxifen; an anti-progesterone such as onapristone and an anti-androgen such as flutamide, in dosages known for such molecules. It also can be beneficial to coadminister a cardioprotectant (to prevent or reduce myocardial dysfunction that can be associated with therapy) or one or more cytokines. In addition to the above therapeutic regimes, the patient can be subjected to surgical removal of cancer cells and/or radiation therapy.

Adjuvants and other immune modulators can be used in combination with CSR isoforms in treating cancers, for example to increase immune response to tumor cells. Combination therapy can increase the effectiveness of treatments and in some cases, create synergistic effects such that the combination is more effective than the additive effect of the treatments separately. Examples of adjuvants include, but are not limited to, bacterial DNA, nucleic acid fraction of attenuated mycobacterial cells (BCG; Bacillus-Calmette-Guerin), synthetic oligonucleotides from the BCG genome, and synthetic oligonucleotides containing CpG motifs (CpG ODN; Wooldridge et al. (1997) Blood 89:2994-2998), levamisole, aluminum hydroxide (alum), BCG, Incomplete Freud's Adjuvant (IFA), QS-21 (a plant derived immunostimulant), keyhole limpet hemocyanin (KLH), and dinitrophenyl (DNP). Examples of immune modulators include but are not limited to, cytokines such as interleukins (e.g., 1L-2, IL-3, IL-4, IL-5, IL-6, IL-7, IL-9, IL-10, IL-11, IL-12, IL-13, IL-15, IL-16, IL-17, IL-18, L-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF, interferon, cytokines such as IL-2 and IL-12; and chemotherapy agents such as methotrexate and chlorambucil.

8. Preclinical Studies

Model animal studies can be used in preclinical evaluation of RTK isoforms that are candidate therapeutics. Paremeters that can be assessed include, but are not limited to efficacy and concentration-response, safety, pharmacokinetics, interspecies scaling and tissue distribution. Model animal studies include assays such as described herein as well as those known to one of skill in the art. Animal models can be used to obtain date that then can be extrapolated to human dosages for design of clinical trials and treatments with RTK isoforms. For example, efficacy and concentration-response VEGFR inhibitors in tumor-bearing mice can be extrapolated to human treatment (Mordenti et al., (1999) Toxicol Pathol. January-February;27(1): 14-21) in order to define clinical dosing regimens effective to maintain a therapeutic inhibitor, such as an antibody against VEGFR for human use in the required efficacious range. Similar models and dose studies can be applied to VEGFR isoform dosage determination and translation into appropriate human doses, as well as other techniques known to the skilled artisan. Preclinical safety studies and preclinical pharmacokinetics can be performed, for example in monkeys, mice, rats and rabbits. Pharmacokinetic data from mice, rats and monkeys has been used to predict the pharmacokinetics of the counterpart therapeutic in human using allometric scaling. Accordingly, appropriate dosage information can be determined for the treatment of human pathological conditions, including rheumatoid arthritis, ocular neovascularization and cancer. A humanized version of the anti-VEGF antibody has been employed in clinical trials as an anti-cancer agent (Brem, (1998) Cancer Res. 58(13):2784-92; Presta et al., (1997) Cancer Res. 57(20):4593-9) and such clinical data also can be considered as a reference source when designing therapeutic doses for VEGFR isoforms.

The following examples are included for illustrative purposes only and are not intended to limit the scope of the invention.

### K. EXAMPLES

### Example 1

### Isolation of a Natural IFP Polypeptide Sequence

The ErbB-2 gene is chosen as a target RTK for generation of natural RTK-IFPs. Expressed sequences are obtained for ErbB-2 using publicly available database sequence. The expressed sequences are aligned using AceView and Acembly with an ErbB-2 genomic sequence as a reference, to produced an aligned set of sequences for ErbB-2. A predominant form of ErbB-2 RTK is identified as a 1255 amino acid form (SEQ ID NO: 27).

Domains of ErbB-2 sequences are mapped relative to the aligned set by using Pfam. Four domains are identified in the predominant ErbB-2 form as shown below in TABLE 3:

TABLE 3

| | | |
|---|---|---|
| | Starting | |
| Domain | amino acid | Ending amino acid |

domains of ErbB-2 predominant form

| | | |
|---|---|---|
| Receptor L domain | 52 | 184 |
| Furin-like | 189 | 343 |
| Receptor L-domain | 366 | 496 |
| pkinase | 720 | 977 |

Other mapped regions

| | | |
|---|---|---|
| Signal peptide | 1 | 22 |
| Transmembrane domain | 653 | 675 |
| Transmembrane domain | 772 | 794 |

The aligned set includes a number of alternatively spliced variants encoding isoforms of erbB-2, including IFPs. IFPs are selected which lack at least a portion of the kinase domain are selected. One exemplary IFP selected is SEQ ID NO: 9. This sequence contains a receptor L domain at amino acids 1-52 and a furin-like domain at 189-343. The c-terminal region encodes 79 amino acids which do not match any of the amino acid sequence in the predominant form of ErbB-2 (SEQ ID NO: 27).

Another exemplary IFP selected is SEQ ID NO: 5. This sequence contains a receptor L domain at amino acids 1-52, a furin-like domain at 189-343, and a second receptor L domain at 366-496. The sequence lacks a transmembrane domain and a protein kinase domain. This IFP shares the first 650 N-terminal amino acids in common with the predominant form of ErbB-2 (SEQ ID NO: 27)and has an additional 30 intron-encoded amino acids which do not have significant sequence similarity with the predominant form of ErbB-2.

Another exemplary IFP selected is SEQ ID NO: 6. This sequence contains a receptor L domain at amino acids 1-52, a furin-like domain at 189-343, and a second receptor L domain at amino acids 366-496. This sequence lacks a transmembrane domain and a kinase domain. This IFP shares the first 633 N-terminal amino acids in common with the predominant form of ErbB-2 (SEQ ID NO: 27) and terminates in a stop codon at the exon/intron boundary, with no additional intron-encoded amino acids.

Another exemplary IFP selected is SEQ ID NO: 7. This sequence contains a receptor L domain at amino acids 1-52, a furin-like domain at 189-343, and a second receptor L domain at amino acids 366-496. This sequence lacks a transmembrane domain and a kinase domain. This IFP shares the first 504 N-terminal amino acids in common with the predominant form of ErbB-2 (SEQ ID NO: 27) and also contains an additional 70 intron-encoded amino acids that lack significant sequence similarity with SEQ ID NO: 27.

### Example 2

### Generation of a Combinatorial IFP

A combinatorial IFP was constructed using the RTK TIE receptor. Expressed TIE gene sequences are aligned with a reference TIE genomic sequence using Acembly (NCBI). The aligned sequences are used to identify introns, exons and intron/exon boundaries in TIE. Domains of TIE sequences are mapped using the Pfam program. TIE domains for the predominant form of TIE (SEQ ID NO:28) are shown below in TABLE 4:

TABLE 4

| | | |
|---|---|---|
| | Starting | |
| Domain | amino acid | Ending amino acid |

TIE domains

| | | |
|---|---|---|
| Pfam-B-30271 | 1 | 40 |
| Pfam-B-7972 | 54 | 138 |
| Ig | 139 | 197 |
| EGF | 224 | 255 |
| EGF | 315 | 344 |
| Ig | 365 | 428 |
| Fn3 | 446 | 533 |
| Fn3 | 546 | 632 |
| Fn3 | 644 | 729 |
| Pfam-B-5918 | 730 | 838 |
| pkinase | 839 | 1107 |

Other mapped regions

| | | |
|---|---|---|
| Signal peptide | 1 | 21 |
| Transmembrane domain | 764 | 786 |

TIE combinatorial IFPs are constructed. SEQ ID NO: 29 is constructed from amino acids 1-838, lacking amino acids 839-1107 of the kinase domain. Additional TIE IFPs are constructed containing amino acids 1-786, 1-632, 1-533, 1-428, 1-344, 1-255 and 1-197 SEQ ID NOS: 25 and 30-35.

Back-translation is used to generate a nucleic acid molecule (SEQ ID NO: 36) encoding TIE 786 IFP. The Backtranslate utility program (Swiss Institute of Bioinformatics; available on the World Wide Web at the URL “us.expasy.org”).

### Example 3

### IFP Cloning using RT-PCR

This example illustrates IFP cloning by RT-PCR with an exemplary IFP from an example gene containing four exons interspersed with three introns. In the example gene, a wildtype or predominant form of the encoded polypeptide is expressed from an RNA containing all four exons with the three introns removed by splicing. Thus, the example gene has the structure E₁-I₁-E₂-I₂-E₃-1₃-E₄, where Eₙ represents an exon and Iₙ represents an intron.

PCR primers are designed to amplify an IFP that is expressed from an RNA that contains all four exons and retains intron 3 (13). PCR primers are designed containing one primer (P1) in E1 and another primer (P3) in 13, such that PCR with P1 and P3 primers amplifies only nucleic acid molecules that contain exon 1 sequence and intron 3 sequence. Primers are designed using a bioinformatics program by Rozen S, Skaletsky H. Primer3 on the internet for general users and for biologist programmers (Methods Mol Biol 2000; 132:365-386). RT-PCR amplification using PCR primers P1 and P3 amplifies only RNA splice variants containing retained intron 3 and not an RNA encoding the wildtype or predominant form. The genomic DNA is not amplified efficiently in most cases and is distinguished from amplification of alternatively spliced RNAs by its larger size amplification product.

Amplified products are confirmed with a second PCR reaction using PCR primers P2 and P3. Primer P2 is designed to hybridize to exon 2 sequence. PCR with primers P2 and P3 generates an amplification product that differs in size between an RNA encoding an IFP and retaining intron 3 as compared to an RNA that does not retain intron 3, such as an RNA encoding the wildtype or predominant form.

A nucleic acid molecule encoding MET (SEQ ID NO: 19) is amplified with primer P1 5′-CGCTGACTTCTCCACTGGTT-3′(SEQ ID NO: 40) and P3 5′-TGAGCCAAAACCCACACATA-3′ (SEQ ID NO: 41) to produce a PCR product of 2890 nucleotides. Confirmation with primers P2 5′-CCAGAAGTGATTGTGGAGCA-3′ (SEQ ID NO: 42) and P3 (SEQ ID NO: 41) produces a product of 1380 nucleotide product. When both products of expected molecular weight are obtained from the separate PCR reactions, amplification of an intron retention splice form has been successful and is confirmed with sequencing.

A nucleic acid molecule encoding FLT1.c BUILD 32 5/24 Proline (SEQ ID NO: 14) is amplified with primer P1 5′-GGGGAAGTGGTTGTCTCCTG-3′(SEQ ID NO: 43) and P35′-GAAACCCATTTGGCACATCT-3′ (SEQ ID NO: 44) to produce a PCR product of 1228 nucleotides. Confirmation with primers P2 5′-GCTTCTGACCTGTGAAGCAA-3′ (SEQ ID NO: 45) and P3 (SEQ ID NO: 44) produces a product of 471 nucleotide product. When both products of expected molecular weight are obtained from the separate PCR reactions, amplification of an intron retention splice form has been successful and is confirmed with sequencing.

A nucleic acid molecule encoding PDGFRA.cDecO3 (SEQ ID NO: 21) is amplified with primer P1 5′-CTCCATGTGTGGGACATTCA-3′(SEQ ID NO: 46) and P3 5′-GGGTCCTAAATCCCCAAATC-3′ (SEQ ID NO: 47) to produce a PCR product of 817 nucleotides. Confirmation with primers P2 5′-CCCACACAGGGTTGTACACTT A-3′ (SEQ ID NO: 48) and P3 (SEQ ID NO: 47) produces a product of 483 nucleotide product. When both products of expected molecular weight are obtained from the separate PCR reactions, amplification of an intron retention splice form has been successful and is confirmed with sequencing.

A nucleic acid molecule encoding Erbb2.dDecO3 (SEQ ID NO: 5) is amplified with primer P1 5′-GTTGCCACTCCCAGACTTGT-3′(SEQ ID NO: 49) and P3 5′-CCTCCCTACAGCAGTGACCA-3′ (SEQ ID NO: 50) to produce a PCR product of 2331 nucleotides. Confirmation with primers P2 5′-ACACAGCGGTGTGAGAAGTG-3′ (SEQ ID NO: 51) and P3 (SEQ ID NO: 50) produces a product of 1047 nucleotide product. When both products of expected molecular weight are obtained from the separate PCR reactions, amplification of an intron retention splice form has been successful and is confirmed with sequencing.

### Example 4

### Method for Cloning RTK Isoforms

A. Preparation of Messenger RNA

mRNAs that represent major human tissue types from healthy or diseased tissues and from cell lines are purchased (e.g. from Clontech (BD Biosciences, Clontech, Palo Alto, Calif.), Stratagene (La Jolla, Calif.), and other commercial providers) and pooled together. This mRNA pool is used as a template for reverse transcription-based PCR amplification (RT-PCR).

B. cDNA Synthesis

mRNA is denatured at 70° C. in the presence of 40% DMSO for 10 min and quenched on ice. First-stand cDNA is synthesized with either 200 ng oligo(dT) 12-16 or 20 ng random hexamers in a 20-μl reaction containing 10% DMSO, 50 mM Tris-HCl (pH 8.3), 75 mM KCl, 3 mM MgCl₂, 10 mM DTT, 2 mM each dNTP, 5 mg mRNA, and 200 units of STRATASCRIPT reverse transcriptase (Stratagene, La Jolla, Calif.). After incubation at 37° C. for 1 h, the cDNA from both reactions are pooled and treated with 10 units of RNase H (Promega, Madison, Wis.).

C. PCR Amplification

Gene-specific PCR primers were selected using the Oligo 6.6 software (Molecular Biology Insights, Inc., Cascade, Colo.). The forward primers flank the start codon. The reverse primers flank the stop codon or are chosen from regions at least 1.5 kb downstream from the start codon. Primers are synthesized by Qiagen (Richmond, Calif.). Each PCR reactions contains 10 ng of reverse-transcribed cDNA, 0.025 u/μl TagPlus (Stratagene), 0.0035 u/μl PfuTurbo® (Stratagene), 0.2 mM dNTP (Amersham, Piscataway, N.J.), and 0.2 μM forward and reverse primers in a total volume of 50 μl. PCR conditions are 35 cycles and 94.5° C. for 45 s, 60° C. for 50 s, and 72° C. for 5 min. The reaction is terminated with an elongation step of 72° C. for 10 min. Exemplary primers for FGFR4 (SEQ ID NO: 53) are set forth in SEQ ID NOs: 38 and 39.

D. Cloning and Sequencing of PCR Products

PCR products are electrophoresed on a 1% agarose gel, and DNA from detectable bands is stained with Gelstar (BioWhitaker Molecular Application, Walkersville, Md.) The DNA bands are extracted with the QiaQuick® gel extraction kit (Qiagen, Valencia, Calif.), ligated into the pDrive UA-cloning vector (Qiagen), and transformed into Escherichia coli. Recombinant plasmids are selected on LB agar plates containing 100 μg/ml carbenicillin. For each transfection, 192 colonies are randomly picked and their cDNA insert sizes are determined by PCR with M13 forward and reverse vector primers. Representative clones from PCR products with distinguishable molecular masses as visualized by fluorescence imaging (Alpha Innotech, San Leandro, Calif.) are then sequenced from both directions with vector primers (M13 forward and reverse). All clones are sequenced entirely using custom primers for directed sequencing completion across gapped regions.

E. Sequence analysis

Computational analysis of alternative splicing is performed by alignment of each cDNA sequence to its respective genomic sequence using SIM4 (a computer program for analysis of splice variants). Only transcripts with canonical (e.g. GT-AG) donor-acceptor splicing sites are considered for analysis. Clones encoding putative RTK isoforms are studied further (see below). F. Targeted cloning Computational analysis of public EST databases can identify potential splice variants with intron retention or insertion. Cloning of potential splice variants identified by EST database analysis can be performed by RT-PCR using primers flanking the putative open reading frame as described above.

### Example 5

### RTK Isoform expression Assays

A. Analysis of mRNA Expression

Expression of the cloned RTK isoforms is determined by RT-PCR (or quantitative PCR) in various tissues using the variant-specific primers (such as set forth in Example 3, TABLE 6).

B. Secretion

Putative RTK isoforms are analyzed in cultured human cells to assess for secreted isoforms. Splice variant cDNAs encoding candidate RTK isoforms are subcloned into a mammalian expression vector, such as the pcDNA3 vector (Invitrogen, Carlsbad, Calif.) with a myc tag fused at the C-terminus of the proteins to facilitate their detection. The recombinant cDNA constructs are transiently transfected into the human embryonic kidney 293 cell. Cell culture supernatant is collected 48 hrs after transfection. Expression of the secreted RTK isoforms in cell culture media is detected by Western blotting with the anti-Myc antibody.

C. Receptor Binding

Binding of RTK isoforms and secreted RTK isoforms to their respective membrane anchored full-length receptor is determined through co-immunoprecipitation experiment (see for example, Jin et al. J Biol Chem 2004, 279:1408 and Jin et al. J Biol Chem 2004, 279:14179).

1. An isolated polypeptide, comprising a sequence of amino acids that has at least 95% sequence identity with a sequence of amino acids set forth in any of SEQ ID NOS: 1, 3, 5-8, 12, 14-17, 19, and 22-25 and allelic variations thereof, wherein:
sequence identity is compared along the full length of each SEQ ID to the full length sequence of the isolated polypeptide; and
each of SEQ ID NOS: 1, 3, 5-8, 12, 14-17, 19 and 22-25 is a receptor tyrosine kinase isoform.

2. An isolated polypeptide, comprising a sequence of amino acids set forth in any of SEQ ID NOs: 1, 3, 5, 7, 8, 12, 14, 15, 16, 17, 19, 22, 23 and 24.

3. The isolated polypeptide of claim 1, wherein the polypeptide contains the same number of amino acids as set forth in the SEQ ID to which it has identity.

4. The isolated polypeptide of claim 1, wherein the polypeptide is from a mammal.

5. The isolated polypeptide of claim 4, wherein the mammal is a rodent, a primate or a human.

6. An isolated polypeptide, comprising at least one domain of a receptor tyrosine kinase operatively linked to at least one amino acid encoded by an intron of a gene encoding the receptor tyrosine kinase,
wherein the receptor tyrosine kinase is selected from the group consisting of DDR, EPHA, FGFR4, MET, PDGFRA, TEK and TIE; or
wherein the polypeptide comprises a sequence of amino acids selected from the group consisting of SEQ ID NOS: 1, 3, 4-8, 10, 12, 14-17, 19, 20, 21 and 22-25.

7. The isolated polypeptide of claim 6, wherein the receptor tyrosine kinase is selected from DDR1, EPHA1 or EPHA8.

8. An isolated polypeptide, comprising a shortened receptor tyrosine kinase lacking at least all or part of a kinase domain and/or all or a part of a transmembrane domain, wherein:
the polypeptide has reduced kinase activity and/or is not membrane localized compared to the non-shortened receptor tyrosine kinase;
the polypeptide modulates a biological activity of the receptor tyrosine kinase;
the receptor tyrosine kinase is selected from the group consisting of DDR, EPHA1, EPHA8, FGFR2, FGFR4, MET, PDGFRA, and TIE, or the isolated polypeptide has at least 95% sequence identity with a sequence of amino acids set forth in any of SEQ ID NOS: 1, 3, 4-8, 10, 11, 12, 14-17, 19, 20, 21 or 22-25; and
sequence identity is compared along the full length of each SEQ ID to the sequence of the full length of the isolated polypeptide.

9. An isolated polypeptide, comprising an intron-encoded sequence of amino acids, wherein:
the intron is from a receptor tyrosine kinase gene selected from the group consisting of DDR1, EGFR, ERBB3, FLT1, MET, PDGFRA, TEK and TIE; or
the intron-encoded sequence of any of SEQ ID NOS: 1-8 and 10-25; and
the polypeptide lacks a receptor tyrosine kinase cytoplasmic domain.

10. The polypeptide of claim 9, wherein the polypeptide further lacks a transmembrane domain.

11. The isolated polypeptide of claim 9, wherein the isolated polypeptide modulates a biological activity of a receptor tyrosine kinase.

12. A pharmaceutical composition, comprising a polypeptide of claim 6.

13. A pharmaceutical composition, comprising a polypeptide, wherein:
the polypeptide comprises a sequence of amino acids that has at least 95% sequence identity with a sequence of amino acids set forth in any of SEQ ID NOS: 1, 3, 4-8, 10, 12, 14-17, 19, 20, 21 and 22-25 and allelic variations thereof;
sequence identity is compared along the full length of each SEQ ID to the full length of the sequence of the isolated polypeptide; and
each of SEQ ID NOS: 1, 3, 4-8, 10, 11, 12, 14-17, 19, 20, 21 and 22-25 is a receptor tyrosine kinase isoform.

14. The composition of claim 12, comprising an amount of the polypeptide effective for modulating a biological activity of a receptor tyrosine kinase.

15. The composition of claim 14, wherein the biological activity of the receptor tyrosine kinase modulated by the polypeptide is one or more of dimerization, homodimerization, heterodimerization, kinase activity, autophosphorylation of the receptor tyrosine kinase, transphosphorylation of the receptor tyrosine kinase, phosphorylation of a signal transduction molecule, ligand binding, competition with the receptor tyrosine kinase for ligand binding, signal transduction, interaction with a signal transduction molecule, membrane association and membrane localization.

16. The composition of claim 15, wherein modulation is an inhibition of activity.

17. The composition of claim 12, wherein the polypeptide of the composition complexes with a receptor tyrosine kinase.

18. A nucleic acid molecule encoding a polypeptide of claim 1.

19. The nucleic acid molecule of claim 18, comprising an intron and an exon, wherein:
the intron contains a stop codon;
the nucleic acid molecule encodes an open reading frame that spans an exon intron junction; and
the open reading frame terminates at the stop codon in the intron.

20. The nucleic acid molecule of claim 19, wherein the intron encodes one or more amino acids of the encoded polypeptide.

21. The nucleic acid molecule of claim 19, wherein the stop codon is the first codon in the intron.

22. A vector, comprising the nucleic acid molecule of claim 18.

23. A cell, comprising the vector of claim 22.

24. A method of treating a disease or condition, comprising administering a pharmaceutical composition of claim 12.

25. The method of claim 24, wherein the disease or condition is selected from the group consisting of cancers, inflammatory diseases, infectious diseases, angiogenesis-related condition, cell proliferation-related conditions, immune disorders and neurodegenerative diseases.

26. The method of claim 24, wherein the disease or condition is selected from the group consisting of rheumatoid arthritis, multiple sclerosis and posterior intraocular inflammation, uveitic disorders, ocular surface inflammatory disorders, neovascular disease, proliferative vitreoretinopathy, atherosclerosis, rheumatoid arthritis, hemangioma, diabetes mellitus, inflammatory bowel disease, Chrohn's disease, psoriasis, Alzheimer's disease, lupus, vascular stenosis, restenosis, inflammatory joint disease, atherosclerosis, urinary obstructive syndromes, and asthma.

27. The method of claim 24, wherein the disease or condition is selected from the group consisting of carcinoma, lymphoma, blastoma, sarcoma, and leukemia, lymphoid malignancies, squamous cell cancer, small-cell lung cancer, non-small cell lung cancer, adenocarcinoma of the lung, squamous carcinoma of the lung, cancer of the peritoneum, hepatocellular cancer, gastric cancer, stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney/renal cancer, prostate cancer, vulval cancer, thyroid cancer, hepatic carcinoma, anal carcinoma, penile carcinoma, and head and neck cancer.

28. The method of claim 24, wherein the disease or condition is a viral or parasitic infection.

29. The method of claim 28, wherein the infection is malaria.

30. The method of claim 29, wherein the pharmaceutical composition comprises a polypeptide that has at least 95% sequence identity with a sequence of amino acids set forth in SEQ ID NO: 19.

31. The method of claim 24, wherein the pharmaceutical composition inhibits angiogenesis, cell proliferation, cell migration, or tumor cell growth or tumor cell metastasis.

32. A method of drug discovery for identifying candidate molecules that modulate the activity of a cell surface receptor, comprising:
a) selecting a set of expressed gene sequences encoding a cell surface receptor or a portion thereof;
b) assembling the set of expressed gene sequences into an aligned set of sequences; and
c) selecting at least one member sequence of the aligned set that encodes a cell surface receptor isoform, wherein the isoform lacks at least one domain or a portion thereof sufficient to modulate a biological activity of the cell surface receptor compared to a wildtype or predominant form of the cell surface receptor, thereby identifying a candidate molecule that modulates the cell surface receptor.

33. The method of claim 32, further comprising:
designating one or more introns and exons within the member sequences of the aligned set by comparing the aligned set with a reference gene sequence; and
selecting at least one member sequence encoding an isoform, wherein the member sequence comprises at least one amino acid and/or a stop codon encoded within an intron, operatively linked to an exon.

34. The method of claim 32, wherein the isoform is a C-terminal shortened cell surface receptor.

35. The method of claim 32, wherein the selected member sequence(s) also contain a 5′ exon corresponding to a 5′ coding exon of the reference gene sequence.

36. The method of claim 32, wherein the cell surface receptor is a receptor tyrosine kinase.

37. The method of claims 32, wherein the isoform lacks a domain or portion thereof selected from the group consisting of a kinase domain, a transmembrane domain or a combination thereof.

38. The method of claim 32, wherein the candidate molecule dimerizes with the cell surface receptor.

39. The method of claim 32, wherein the candidate molecule binds a ligand and wherein, the cell surface receptor binds the same ligand.

40. The method of claim 32, wherein the candidate molecule competes with the cell surface receptor for ligand binding.

41. The method of claims 32, wherein the candidate molecule inhibits phosphorylation of the cell surface receptor.

42. The method of claim 32, wherein the candidate molecule is modified in a biological activity of the cell surface receptor.

43. The method of claim 42, wherein the modified biological activity is selected from the group consisting of dimerization, kinase activity, signal transduction, ligand binding, membrane association and membrane localization.

44. The method of claim 42, wherein the candidate molecule is reduced in the biological activity as compared to the wildtype or predominant form of the receptor.

45. The method of claim 33, wherein the selected member sequence comprises the addition of at least one amino acid or a stop codon operatively linked to an exon encoding a kinase domain.

46. The method of claim 33, wherein the selected member sequence comprises the addition of at least one amino acid or stop codon operatively linked to an exon encoding a transmembrane domain.

47. A pharmaceutical composition, comprising a polypeptide of claim 8.

48. A pharmaceutical composition, comprising a polypeptide of claim 9.

49. The composition of claim 13, comprising an amount of the polypeptide effective for modulating a biological activity of a receptor tyrosine kinase.

50. A nucleic acid molecule encoding a polypeptide of claim 6.

51. A nucleic acid molecule encoding a polypeptide of claim 8.

52. A nucleic acid molecule encoding a polypeptide of claim 9.


SEQUENCE LISTING (53 sequences)

SEQ ID NO: 1 - protein, 444 aa, Artificial Sequence
  Human DDR1.h
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFCPHLPRTASPIMPRLTLLPCRASPGATPMLCLHCPQGQSGMGPPEWISLDLDSASRRSLARASLGRCTCVRSTALKIWLVLISPLMCVRDTLCW

SEQ ID NO: 2 - protein, 405 aa, Artificial Sequence
  Human EGFR.a
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGLS

SEQ ID NO: 3 - protein, 166 aa, Artificial Sequence
  Human EPHA1.b
MDTSKAQGELGWLLDPPKDGWSEQQQILNGTPLYMYQDCPMQGRRDTDHWLRSNWIYRGEEASRVHVELQFTVRDCKSFPGGAGPLGCKETFNLLYMESDQDVGIQLRRPLFQKVLLPSMPSGSWCRSLVAPYWVPEKVAETGRGCRGRILKRIWRLKAGHGGLCL

SEQ ID NO: 4 - protein, 495 aa, Artificial Sequence
  Human EPHA8.b
MAPARGRLPPALWVVTAAAAAATCVSAARGEVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPAMVRNLAAFSEAVTGADSSSLVEVRGQCVRHSEERDTPKMYCSAEGEWLVPIGKCVCSAGYEERRDACVACELGFYKSAPGDQLCARCPPHSHSAAPAAQACHCDLSYYRAALDPPSSACTRPPSAPVNLISSVNGTSVTLEWAPPLDPGGRSDITYNAVCRRCPWALSRCEACGSGTRFVPQQTSLVQASLLVANLLAHMNYSFWIEAVNGVSDLSPEPRRAAVVNITTNQAGRRRNSVPQRPGPPASPASDPSRDQSSAGDVLWAFRQVPLWPCAPHQDPELEALHCL

SEQ ID NO: 5 - protein, 680 aa, Artificial Sequence
  Human ERBB2.1.d
MELAALCRWGLLLALLPLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRARLAWTPGCTLHCPSLPHWMLGGHCCREGTP

SEQ ID NO: 6 - protein, 633 aa, Artificial Sequence
  Human ERBB2.1.e
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHS

SEQ ID NO: 7 - protein, 575 aa, Artificial Sequence
  Human ERBB2.1.f
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHYTHLCFVHTVPWDQLFRNPHQALLHTANRPEDECGKTGSPVCALPICQHTAVPRGPWQQRSWTCADCPSLCTLLDSAQLWLAWPLGMASLAGSYLPWHPSLPLCF

SEQ ID NO: 8 - protein, 90 aa, Artificial Sequence
  Human ERBB2.a
MELAALCRWGLLLALLPPGAASTQVCTGTDMKLRLPASPETHLDMLRHLYQGCQVVQGNLELTYLPTNASLSFLQVRPVGNPARPCLQLG

SEQ ID NO: 9 - protein, 419 aa, Artificial Sequence
  Human ERBB2.c BUILD 31
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTHSLPPRPAAVPVPLRMQPGPAHPVLSFLRPSWDLVSAFYSLPLAPLSPTSVPISPVSVGRGPDPDAHVAVDLSRYEG

SEQ ID NO: 10 - protein, 331 aa, Artificial Sequence
  Human ERBB3.d BUILD 31
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 11 - protein, 366 aa, Artificial Sequence
  Human FGFR2.b BUILD 31
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQGRRC

SEQ ID NO: 12 - protein, 209 aa, Artificial Sequence
  Human FGFR4.d BUILD 31
MMRTPSPIGTPRIGTVTPSKVSRSPRTCVPAAAHLITEKRRPVWEHTVILGAFPCPPAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGAD

SEQ ID NO: 13 - protein, 479 aa, Artificial Sequence
  Human FLT1.c BUILD 31
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEAR

SEQ ID NO: 14 - protein, 523 aa, Artificial Sequence
  Human FLT1.c BUILD 32
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEILTCTAYGIPQPTIKWFWHPCNHNHSEARCDFCSNNEESFILDADSNMGNRIESITQRMAIIEGKNKLPPANSSFMLPPTSFSSNYFHFLP

SEQ ID NO: 15 - protein, 541 aa, Artificial Sequence
  Human FLT1.c
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKLPPANSSFMLPPTSFSSNYFHFLP

SEQ ID NO: 16 - protein, 436 aa, Artificial Sequence
  Human FLT1.c BUILD 33
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDKNLTATLIVNVKPQERIRERISPDLYRIWYPSTYNQVVLAPL

SEQ ID NO: 17 - protein, 365 aa, Artificial Sequence
  Human FLT1.c BUILD34
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYGKHSSALPTHAMLSNHCRCLCSLNKSVFCWPRVTLS

SEQ ID NO: 18 - protein, 687 aa, Artificial Sequence
  Human FLT1.d BUILD 31
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 19 - protein, 934 aa, Artificial Sequence
  Human MET.f  BUILD 34
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 20 - protein, 217 aa, Artificial Sequence
  Human PDGFRA.b BUILD 31
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKGTCIISFL

SEQ ID NO: 21 - protein, 218 aa, Artificial Sequence
  Human PDGFRA.c  BUILD34
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKGTCIISFLL

SEQ ID NO: 22 - protein, 798 aa, Artificial Sequence
  Human TEK.c BUILD 31
MDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEGIQRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHR

SEQ ID NO: 23 - protein, 821 aa, Artificial Sequence
  Human TEKc BUILD34
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEGIQRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHR

SEQ ID NO: 24 - protein, 864 aa, Artificial Sequence
  Human TEKc
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIQRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHR

SEQ ID NO: 25 - protein, 786 aa, Artificial Sequence
  Human TIE. IFP
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRVSTSGGQDSRRFKVNVKVPPVPLAAPRLLTKQSRQLVVSPLVSFSGDGPISTVRLHYRPQDSTMDWSTIVVDPSENVTLMNLRPKTGYSVRVQLSRPGEGGEGAWGPPTLMTTDCPEPLLQPWLEGWHVEGTDRLRVSWSLPLVPGPLVGDGFLLRLWDGTRGQERRENVSSPQARTALLTGLTPGTHYQLDVQLYHCTLLGPASPPAHVLLPPSGPPAPRHLHAQALSDSEIQLTWKHPEALPGPISKYVVEVQVAGGAGDPLWIDVDRPEETSTIIRGLNASTRYLFRMRASIQGLGDWSNTVEESTLGNGLQAEGPVQESRAAEEGLDQQLILAVVGSVSATCLTILAALLTLVCI

SEQ ID NO: 26 - DNA, 2802 bp, Artificial Sequence
  Human MET.f
atgaaagccccagctgttttggctccaggtattttggtattgctgttcaccctggtccaaagatctaacggagaatgtaaagaggctttagctaagtcagaaatgaatgtcaatatgaagtatcagctgccaaatttcactgccgagacacccattcagaatgtcatccttcacgaacatcacattttcttaggtgctactaactacatctatgtcttaaacgaggaagatcttcagaaagtggccgagtacaaaacgggcccagttttggaacaccctgactgtttcccttgtcaagactgctcatctaaagcgaatttatctggaggtgtctggaaggacaacattaatatggctttggtagttgatacctactatgatgaccaattgatttcatgcggctctgtcaatcgtggtacatgccagcgtcatgttttcccccataatcatacggcagatattcagtccgaggttcactgcattttttctcctcaaatagaggaaccttcccaatgcccggattgcgtcgtgtccgctttgggagccaaggtcttgtcatcggttaaagatagatttatcaactttttcgttggtaatacaatcaactcgtcttactttccagatcacccattgcatagtatttctgttagacgacttaaggagaccaaggacgggttcatgtttctgaccgaccaaagctatatcgatgtgttacctgaattcagggattcatatccaataaaatatgtgcatgcatttgagtctaataactttatatattttctaactgtccagcgtgaaactttagatgcccaaacctttcatactagaataattcggttctgttctattaatagtggcttgcattcgtacatggagatgccattagaatgtattctaactgaaaagagaaagaaaagatccaccaaaaaggaagttttcaatattcttcaagccgcttacgtttccaagcccggagcccagcttgctagacagattggtgcatctttaaacgatgacatcttattcggtgttttcgctcagtccaagccagattcagctgagcccatggacagatctgccatgtgtgcttttccaataaagtatgttaacgatttctttaataaaattgtaaacaaaaataacgttcgttgtttacaacacttttacggtcctaaccacgaacattgttttaaccgaactttgctaaggaattcctctgggtgtgaagctaggcgggacgagtaccgtacagagtttacgaccgcgctccaaagagtggacttattcatgggtcaattctctgaagtgttgctaacttccattagtacttttattaagggtgacttgactattgccaacctgggtacctccgaagggcgatttatgcaagtcgttgtctctagatccggtccatctactccacacgtaaacttccttttggattcacaccctgttagcccagaggtcatcgttgaacataccctgaaccagaacggatacacacttgttattacaggtaagaaaattaccaagattcctctcaatggattgggatgccgtcattttcaaagctgctcacaatgtttgagcgcacctcccttcgtgcaatgtggatggtgccatgataaatgtgttaggtcagaagagtgtctctctggcacttggactcagcaaatatgtctaccggcaatctataaagtcttccccaattcagcaccattggagggtggaacaagattgacaatttgcggctgggattttggctttcgaagaaacaataagttcgacctcaagaaaacgagagtgttgctggggaacgagtcttgtactctaactctgtcggaatccaccatgaacacattgaagtgtactgttggacctgctatgaacaaacatttcaacatgagtatcattatctcaaacggacatggtactacacaatacagcacctttagttatgtggacccagttattacttcaataagtccaaaatatggaccgatggctggtggaacgcttttgacactgacaggtaattatcttaacagtggaaactcgaggcatatttcgataggtggaaagacctgcacacttaaatctgttagtaattccatcctggaatgttacactcctgcacaaacaatttcaactgagtttgcagtaaaacttaagatcgatctagcaaacagagaaacgtctattttttcgtacagagaagatccgatagtctacgagatccaccctactaagtcatttatttccggtgggagcactatcactggtgtgggaaaaaacctgaatagtgttagtgttccaagaatggtaataaacgttcatgaggctggtcgcaattttactgttgcatgtcaacaccgctccaattctgaaattatctgttgtaccacaccttccctgcagcaacttaatctccagttacctttgaagaccaaggctttctttatgttggacggtatcttaagtaagtactttgatttgatttacgtccacaaccctgtatttaaacctttcgaaaagccagtaatgatttctatgggaaacgaaaacgttctcgaaatcaaggggaatgatattgaccccgaggccgtgaagggtgaagtgctgaaagtaggaaacaaaagttgtgaaaatatccacttgcatagcgaagctgtactatgtactgtcccaaatgaccttttgaaattgaattctgagctaaacatagaagttggttttctacactcttcccacgatgtgaataaggaggcctcagttatcatgttgttctcaggccttaag

SEQ ID NO: 27 - protein, 1255 aa, Artificial Sequence
  Human ERBB2 RTK
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARCHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 28 - protein, 1138 aa, Artificial Sequence
  Human TIE
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRVSTSGGQDSRRFKVNVKVPPVPLAAPRLLTKQSRQLVVSPLVSFSGDGPISTVRLHYRPQDSTMDWSTIVVDPSENVTLMNLRPKTGYSVRVQLSRPGEGGEGAWGPPTLMTTDCPEPLLQPWLEGWHVEGTDRLRVSWSLPLVPGPLVGDGFLLRLWDGTRGQERRENVSSPQARTALLTGLTPGTHYQLDVQLYHCTLLGPASPPAHVLLPPSGPPAPRHLHAQALSDSEIQLTWKHPEALPGPISKYVVEVQVAGGAGDPLWIDVDRPEETSTIIRGLNASTRYLFRMRASIQGLGDWSNTVEESTLGNGLQAEGPVQESRAAEEGLDQQLILAVVGSVSATCLTILAALLTLVCIRRSCLHRRRTFTYQSGSGEETILQFSSGTLTLTRRPKLQPEPLSYPVLEWEDITFEDLIGEGNFGQVIRAMIKKDGLKMNAAIKMLKEYASENDHRDFAGELEVLCKLGHHPNIINLLGACKNRGYLYIAIEYAPYGNLLDFLRKSRVLETDPAFAREHGTASTLSSRQLLRFASDAANGMQYLSEKQFIHRDLAARNVLVGENLASKIADFGLSRGEEVYVKKTMGRLPVRWMAIESLNYSVYTTKSDVWSFGVLLWEIVSLGGTPYCGMTCAELYEKLPQGYRMEQPRNCDDEVYELMRQCWRDRPYERPPFAQIALQLGRMLEARKAYVNMSLFENFTYAGIDATAEEA

SEQ ID NO: 29 - protein, 838 aa, Artificial Sequence
  Human TIE.IFP
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRVSTSGGQDSRRFKVNVKVPPVPLAAPRLLTKQSRQLVVSPLVSFSGDGPISTVRLHYRPQDSTMDWSTIVVDPSENVTLMNLRPKTGYSVRVQLSRPGEGGEGAWGPPTLMTTDCPEPLLQPWLEGWHVEGTDRLRVSWSLPLVPGPLVGDGFLLRLWDGTRGQERRENVSSPQARTALLTGLTPGTHYQLDVQLYHCTLLGPASPPAHVLLPPSGPPAPRHLHAQALSDSEIQLTWKHPEALPGPISKYVVEVQVAGGAGDPLWIDVDRPEETSTIIRGLNASTRYLFRMRASIQGLGDWSNTVEESTLGNGLQAEGPVQESRAAEEGLDQQLILAVVGSVSATCLTILAALLTLVCIRRSCLHRRRTFTYQSGSGEETILQFSSGTLTLTRRPKLQPEPLSYPVLEWED

SEQ ID NO: 30 - protein, 632 aa, Artificial Sequence
  Human TIE.IFP
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRVSTSGGQDSRRFKVNVKVPPVPLAAPRLLTKQSRQLVVSPLVSFSGDGPISTVRLHYRPQDSTMDWSTIVVDPSENVTLMNLRPKTGYSVRVQLSRPGEGGEGAWGPPTLMTTDCPEPLLQPWLEGWHVEGTDRLRVSWSLPLVPGPLVGDGFLLRLWDGTRGQERRENVSSPQARTALLTGLTPGTHYQLDVQLYHCTLLGPAS

SEQ ID NO: 31 - protein, 533 aa, Artificial Sequence
  Human TIE.IFP
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRVSTSGGQDSRRFKVNVKVPPVPLAAPRLLTKQSRQLVVSPLVSFSGDGPISTVRLHYRPQDSTMDWSTIVVDPSENVTLMNLRPKTGYSVRVQLSRPGEGGEGAWG

SEQ ID NO: 32 - protein, 428 aa, Artificial Sequence
  Human TIE.IFP
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRV

SEQ ID NO: 33 - protein, 344 aa, Artificial Sequence
  Human TIE.IFP
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHC

SEQ ID NO: 34 - protein, 255 aa, Artificial Sequence
  Human TIE.IFP
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRC

SEQ ID NO: 35 - protein, 197 aa, Artificial Sequence
  Human TIE.IFP
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATY

SEQ ID NO: 36 - DNA, 2358 bp, Artificial Sequence
  Human TIE.IFP
atggtatggcgtgtgccgccctttctcttaccgatcctgttcctagcctctcatgtcggagcagccgtggaccttacattattggctaacctccggctgactgatccccagcgctttttcctaacatgcgtaagtggtgaagctggtgcagggcggggctcggatgcttggggccccccacttttgttagaaaaagacgatcgtatcgtaaggacaccaccaggcccaccccttagactcgctcgaaacggaagtcatcaagtaactctacgtgggttttccaaaccttcggatcttgtcggcgtgtttagttgtgtgggaggcgccggagcgcgccgaactcgagttatatacgtacataattcgccaggcgcgcacttacttccagataaggtcacccacacggtcaataaaggtgacacggcagtgctgagcgctcgggtgcataaggaaaagcagaccgacgtcatttggaaatcgaacgggtcttatttttatacgctcgactggcatgaggcccaggacggccgatttttgctgcagttacccaatgttcagcctcccagttcggggatatattcagccacttatctggaggcatcacccctgggctctgcattcttcaggcttatcgtacgggggtgcggagcgggcaggtggggtccggggtgtaccaaagagtgcccagggtgcctgcacgggggtgtttgtcacgatcacgacggtgagtgcgtatgcccacctggattcaccggaactcgctgcgagcaagcctgcagagagggccgatttgggcaaagttgccaagagcagtgtcccgggatatccggttgtagaggacttactttctgtctaccagatccttacggatgctcgtgcggctcgggctggcggggtagtcaatgtcaggaagcgtgtgcccctggtcactttggcgcggactgccgtctccagtgtcaatgtcaaaacggtggcacgtgtgatagattttccggctgcgtatgtccttctggttggcacggagttcactgcgagaaaagcgatcgaattcctcaaatattaaacatggcatctgagctggaattcaatctagaaacaatgccgcggattaattgtgccgctgcaggaaatccatttcctgtccgcgggagtatcgagctcaggaagccagatggaaccgtattattgagtacgaaggcaatagtggagcctgaaaagactacagcagaattcgaggttccgagattggtccttgccgacagcggcttctgggagtgcagagtcagcactagtggtggtcaggattcgcgcaggttcaaagtaaacgtcaaagttcctccggttcctttggcggcgcctcgcctgttaactaagcagagcagacaactcgttgtctcaccacttgtgagcttctctggggatgggccgatatccacagtaaggcttcattacagaccccaggacagtactatggactggtctaccattgttgtagacccatccgaaaacgtcacgctaatgaacttgagaccgaagacgggttactcagtgcgggtgcagttgtcccgcccgggggaaggtggagaaggggcttggggccctccaacactaatgacgactgattgtcccgaacccctcctgcaaccgtggctcgagggttggcatgttgaagggaccgacaggttaagagtgagctggtctctacccctagtcccaggaccattagtaggggatggttttttattacgactttgggacggtacacgaggccaagaacgtcgtgagaatgtgtcatcaccgcaagcaagaaccgcattattaacgggattaacaccaggaacccactatcaattagacgtgcaactgtaccattgtacgctcctaggaccggcatcccccccggcgcatgttctattgccccctagtggtccgccggctccacgccatctacatgcacaggctctctcggattcagagattcagctcacatggaagcaccccgaggctcttcccggccctatctcaaaatatgttgttgaagtgcaggttgccggtggggccggagatccactttggattgatgtcgacaggcctgaagaaacctcaactattatccgggggttgaatgcctccacgagatacttgtttcgtatgcgtgcaagtatacaaggacttggcgactggagcaacacagtcgaagaaagtacactggggaatggattgcaggcggagggaccggtgcaggagagcagagcggcagaggaaggattggatcaacaactaatactagctgttgtaggatctgtctccgcgacctgcctcacgatactcgctgcgctgttgaccctggtatgtatc

SEQ ID NO: 37 - DNA, 3015 bp, Homo sapiens
  FGFR4 NM_002011
ccgaggagcgctcgggctgtctgcggaccctgccgcgtgcaggggtcgcggccggctggagctgggagtgaggcggcggaggagccaggtgaggaggagccaggaaggcagttggtgggaagtccagcttgggtccctgagagctgtgagaaggagatgcggctgctgctggccctgttgggggtcctgctgagtgtgcctgggcctccagtcttgtccctggaggcctctgaggaagtggagcttgagccctgcctggctcccagcctggagcagcaagagcaggagctgacagtagcccttgggcagcctgtgcggctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacctctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgctataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccaggtatacggacatcatcctgtacgcgtcgggctccctggccttggctgtgctcctgctgctggccgggctgtatcgagggcaggcgctccacggccggcacccccgcccgcccgccactgtgcagaagctctcccgcttccctctggcccgacagttctccctggagtcaggctcttccggcaagtcaagctcatccctggtacgaggcgtgcgtctctcctccagcggccccgccttgctcgccggcctcgtgagtctagatctacctctcgacccactatgggagttcccccgggacaggctggtgcttgggaagcccctaggcgagggctgctttggccaggtagtacgtgcagaggcctttggcatggaccctgcccggcctgaccaagccagcactgtggccgtcaagatgctcaaagacaacgcctctgacaaggacctggccgacctggtctcggagatggaggtgatgaagctgatcggccgacacaagaacatcatcaacctgcttggtgtctgcacccaggaagggcccctgtacgtgatcgtggagtgcgccgccaagggaaacctgcgggagttcctgcgggcccggcgccccccaggccccgacctcagccccgacggtcctcggagcagtgaggggccgctctccttcccagtcctggtctcctgcgcctaccaggtggcccgaggcatgcagtatctggagtcccggaagtgtatccaccgggacctggctgcccgcaatgtgctggtgactgaggacaatgtgatgaagattgctgactttgggctggcccgcggcgtccaccacattgactactataagaaaaccagcaacggccgcctgcctgtgaagtggatggcgcccgaggccttgtttgaccgggtgtacacacaccagagtgacgtgtggtcttttgggatcctgctatgggagatcttcaccctcgggggctccccgtatcctggcatcccggtggaggagctgttctcgctgctgcgggagggacatcggatggaccgacccccacactgccccccagagctgtacgggctgatgcgtgagtgctggcacgcagcgccctcccagaggcctaccttcaagcagctggtggaggcgctggacaaggtcctgctggccgtctctgaggagtacctcgacctccgcctgaccttcggaccctattccccctctggtggggacgccagcagcacctgctcctccagcgattctgtcttcagccacgaccccctgccattgggatccagctccttccccttcgggtctggggtgcagacatgagcaaggctcaaggctgtgcaggcacataggctggtggccttgggccttggggctcagccacagcctgacacagtgctcgaccttgatagcatggggcccctggcccagagttgctgtgccgtgtccaagggccgtgcccttgcccttggagctgccgtgcctgtgtcctgatggcccaaatgtcagggttctgctcggcttcttggaccatggcgcttagtccccatcccgggtttggctgagcctggctggagagctgctatgctaaacctcctgcctcccaataccagcaggaggttctgggcctctgaaccccctttccccacacctccccctgctgctgctgccccagcgtcttgacgggagcattggcccctgagcccagagaagctggaagcctgccgaaaacaggagcaaatggcgttttataaattatttttttgaaataaa

SEQ ID NO: 38 - DNA, 20 bp, Artificial Sequence
  Primer FGFR4_F1
agaaggagatgcggctgctg

SEQ ID NO: 39 - DNA, 20 bp, Artificial Sequence
  Primer FGFR4_R1
cgggggaactcccatagtgg

SEQ ID NO: 40 - DNA, 20 bp, Artificial Sequence
  Primer -
cgctgacttctccactggtt

SEQ ID NO: 41 - DNA, 20 bp, Artificial Sequence
  Primer -
tgagccaaaacccacacata

SEQ ID NO: 42 - DNA, 20 bp, Artificial Sequence
  Primer -
ccagaagtgattgtggagca

SEQ ID NO: 43 - DNA, 20 bp, Artificial Sequence
  Primer -
ggggaagtggttgtctcctg

SEQ ID NO: 44 - DNA, 20 bp, Artificial Sequence
  Primer -
gaaacccatttggcacatct

SEQ ID NO: 45 - DNA, 20 bp, Artificial Sequence
  Primer -
gcttctgacctgtgaagcaa

SEQ ID NO: 46 - DNA, 20 bp, Artificial Sequence
  Primer -
ctccatgtgtgggacattca

SEQ ID NO: 47 - DNA, 20 bp, Artificial Sequence
  Primer -
gggtcctaaatccccaaatc

SEQ ID NO: 48 - DNA, 22 bp, Artificial Sequence
  Primer -
cccacacagggttgtacactta

SEQ ID NO: 49 - DNA, 20 bp, Artificial Sequence
  Primer -
gttgccactcccagacttgt

SEQ ID NO: 50 - DNA, 20 bp, Artificial Sequence
  Primer -
cctccctacagcagtgacca

SEQ ID NO: 51 - DNA, 20 bp, Artificial Sequence
  Primer -
acacagcggtgtgagaagtg

SEQ ID NO: 52 - DNA, 713 bp, Artificial Sequence
  PCR Product Sequence for FGFR4
tgggtccctgagagctgtgagaaggagatgcggctgctgctggccctgttgggggtcctgctgagtgtgcctgggcctccagtcttgtccctggaggcctctgaggaagtggagcttgcaagcattcatctatcactgtgtctgcgagagaggactggccttgcagggcgcagggccctaagctgggctgcagagctggtgagccctgcctggctcccagcctggagcagcaagagcaggagctgacagtagcccttgggcagcctgtgcgtctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacccctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcgg

SEQ ID NO: 53 - protein, 72 aa, Artificial Sequence
  FGFR4 Translation
MRLLLALLGVLLSVPGPPVLSLEASEEVELASIHLSLCLRERTGLAGRRALSWAAELVSPAWLPAWSSKSRS